SEQUENCE LISTING (136 sequences)

SEQ ID NO: 1 - protein, 12 aa, synthetic construct
  REGION 1..12: note = PHB2 peptide No. 1
RLPAGPRGMGTA

SEQ ID NO: 2 - protein, 9 aa, synthetic construct
  REGION 1..9: note = PHB2 peptide No. 2
TVEGGHRAI

SEQ ID NO: 3 - protein, 13 aa, synthetic construct
  REGION 1..13: note = PHB2 peptide No. 3
ESVFTVEGGHRAI

SEQ ID NO: 4 - protein, 17 aa, synthetic construct
  REGION 1..17: note = PHB2 peptide No. 4
YGVRESVFTVEGGHRAI

SEQ ID NO: 5 - protein, 15 aa, synthetic construct
  REGION 1..15: note = PHB2 peptide No. 5
QYPIIYDIRARPRKI

SEQ ID NO: 6 - protein, 15 aa, synthetic construct
  REGION 1..15: note = PHB2 peptide No. 6
RPRKISSPTGSKDLQ

SEQ ID NO: 7 - protein, 15 aa, synthetic construct
  REGION 1..15: note = PHB2 peptide No. 7
SKDLQMVNISLRVLS

SEQ ID NO: 8 - protein, 15 aa, synthetic construct
  REGION 1..15: note = PHB2 peptide No. 8
LRVLSRPNAQELPSM

SEQ ID NO: 9 - protein, 15 aa, synthetic construct
  REGION 1..15: note = PHB2 peptide No. 9
ELPSMYQRLGLDYEE

SEQ ID NO: 10 - protein, 15 aa, synthetic construct
  REGION 1..15: note = PHB2 peptide No. 10
LDYEERVLPSIVNEV

SEQ ID NO: 11 - protein, 15 aa, synthetic construct
  REGION 1..15: note = PHB2 peptide No. 11
IVNEVLKSVVAKFNA

SEQ ID NO: 12 - protein, 15 aa, synthetic construct
  REGION 1..15: note = PHB2 peptide No. 12
AKFNASQLITQRAQV

SEQ ID NO: 13 - protein, 15 aa, synthetic construct
  REGION 1..15: note = PHB2 peptide No. 13
QRAQVSLLIRRELTE

SEQ ID NO: 14 - protein, 15 aa, synthetic construct
  REGION 1..15: note = PHB2 peptide No. 14
RELTERAKDFSLILD

SEQ ID NO: 15 - protein, 15 aa, synthetic construct
  REGION 1..15: note = PHB2 peptide No. 15
SLILDDVAITELSFS

SEQ ID NO: 16 - protein, 15 aa, synthetic construct
  REGION 1..15: note = PHB2 peptide No. 16
ELSFSREYTAAVEAK

SEQ ID NO: 17 - protein, 15 aa, synthetic construct
  REGION 1..15: note = PHB2 peptide No. 17
AVEAKQVAQQEAQRA

SEQ ID NO: 18 - protein, 15 aa, synthetic construct
  REGION 1..15: note = PHB2 peptide No. 18
EAQRAQFLVEKAKQE

SEQ ID NO: 19 - protein, 15 aa, synthetic construct
  REGION 1..15: note = PHB2 peptide No. 19
QFLVEKAKQEQRQKI

SEQ ID NO: 20 - protein, 14 aa, synthetic construct
  REGION 1..14: note = PHB2 peptide No. 20
FLVEKAKQEQRQKI

SEQ ID NO: 21 - protein, 80 aa, synthetic construct
  REGION 1..80: note = PHB2 derived peptide 11-90aa
RLPAGPRGMGTALKLLLGAGAVAYGVRESVFTVEGGHRAIFFNRIGGVQQDTILAEGLHFRIPWFQYPIIYDIRARPRKI

SEQ ID NO: 22 - protein, 12 aa, synthetic construct
  REGION 1..12: note = PHB2 derived peptide 11-22aa
  MOD_RES 1: note = ACETYLATION
RLPAGPRGMGTA

SEQ ID NO: 23 - protein, 23 aa, synthetic construct
  REGION 1..23: note = PHB2 derived peptide 76-90aa + 8 Arg residues
  MOD_RES 23: note = AMIDATION
QYPIIYDIRARPRKIRRRRRRRR

SEQ ID NO: 24 - protein, 22 aa, synthetic construct
  REGION 1..22: note = PHB2 derived peptide 76-89aa + 8 Arg residues
  MOD_RES 1: note = ACETYLATION
  MOD_RES 22: note = AMIDATION
QYPIIYDIRARPRKRRRRRRRR

SEQ ID NO: 25 - protein, 19 aa, synthetic construct
  REGION 1..19: note = cyclic peptide containing PHB2 derived peptide 11-21aa
  DISULFID 1..13: note = Intrachain disulfide bond
  SITE 10: note = Nle
  MOD_RES 15: note = 2-Naphthylalanine
CRLPAGPRGXGTCFXRRRR

SEQ ID NO: 26 - protein, 21 aa, synthetic construct
  REGION 1..21: note = cyclic peptide containing PHB2 derived peptide 76-88aa
  DISULFID 1..15: note = Intrachain disulfide bond
  MOD_RES 17: note = 2-Naphthylalanine
CQYPIIYDIRARPRCFXRRRR

SEQ ID NO: 27 - DNA, 1457 bp, Homo sapiens
  source 1..1457: mol_type = unassigned DNA
  CDS 211..1110: protein_id = 28; translation = MAQNLKDLAGRLPAGPRGMGTALKLLLGAGAVAYGVRESVFTVE GGHRAIFFNRIGGVQQDTILAEGLHFRIPWFQYPIIYDIRARPRKISSPTGSKDLQMV NISLRVLSRPNAQELPSMYQRLGLDYEERVLPSIVNEVLKSVVAKFNASQLITQRAQV SLLIRRELTERAKDFSLILDDVAITELSFSREYTAAVEAKQVAQQEAQRAQFLVEKAK QEQRQKIVQAEGEAEAAKMLGEALSKNPGYIKLRKIRAAQNISKTIATSQNRIYLTAD NLVLNLQDESFTRGSDSLIKGKK
tccgtatgcgcgattcctgtgcgcgaagttcgggtccgtagtgggctaagggggagggtttcaaagggagcgcacttccgctgccctttctttcgccagccttacgggcccgaaccctcgtgtgaagggtgcagtacctaagccggagcggggtagaggcgggccggcacccccttctgacctccagtgccgccggcctcaagatcagacatggcccagaacttgaaggacttggcgggacggctgcccgccgggccccggggcatgggcacggccctgaagctgttgctgggggccggcgccgtggcctacggtgtgcgcgaatctgtgttcaccgtggaaggcgggcacagagccatcttcttcaatcggatcggtggagtgcagcaggacactatcctggccgagggccttcacttcaggatcccttggttccagtaccccattatctatgacattcgggccagacctcgaaaaatctcctcccctacaggctccaaagacctacagatggtgaatatctccctgcgagtgttgtctcgacccaatgctcaggagcttcctagcatgtaccagcgcctagggctggactacgaggaacgagtgttgccgtccattgtcaacgaggtgctcaagagtgtggtggccaagttcaatgcctcacagctgatcacccagcgggcccaggtatccctgttgatccgccgggagctgacagagagggccaaggacttcagcctcatcctggatgatgtggccatcacagagctgagctttagccgagagtacacagctgctgtagaagccaaacaagtggcccagcaggaggcccagcgggcccaattcttggtagaaaaagcaaagcaggaacagcggcagaaaattgtgcaggccgagggtgaggccgaggctgccaagatgcttggagaagcactgagcaagaaccctggctacatcaaacttcgcaagattcgagcagcccagaatatctccaagacgatcgccacatcacagaatcgtatctatctcacagctgacaaccttgtgctgaacctacaggatgaaagtttcaccaggggaagtgacagcctcatcaagggtaagaaatgagcctagtcaccaagaactccacccccagaggaagtggatctgcttctccagtttttgaggagccagccaggggtccagcacagccctaccccgccccagtatcatgcgatggtcccccacaccggttccctgaacccctcttggattaaggaagactgaagactagccccttttctggggaattactttcctcctccctgtgttaactggggctgttggggacagtgcgtgatttctcagtgatttcctacagtgttgttccctccctcaaggctgggaggagataaacaccaacccaggaattctcaataaatttttattacttaacctgaaaaaaaaaaaaaaaa

SEQ ID NO: 28 - protein, 299 aa, Homo sapiens
MAQNLKDLAGRLPAGPRGMGTALKLLLGAGAVAYGVRESVFTVEGGHRAIFFNRIGGVQQDTILAEGLHFRIPWFQYPIIYDIRARPRKISSPTGSKDLQMVNISLRVLSRPNAQELPSMYQRLGLDYEERVLPSIVNEVLKSVVAKFNASQLITQRAQVSLLIRRELTERAKDFSLILDDVAITELSFSREYTAAVEAKQVAQQEAQRAQFLVEKAKQEQRQKIVQAEGEAEAAKMLGEALSKNPGYIKLRKIRAAQNISKTIATSQNRIYLTADNLVLNLQDESFTRGSDSLIKGKK

SEQ ID NO: 29 - DNA, 1343 bp, Homo sapiens
  source 1..1343: mol_type = unassigned DNA
  CDS 211..996: protein_id = 30; translation = MAQNLKDLAGRLPAGPRGMGTALKLLLGAGAVAYGVRESVFTVE GGHRAIFFNRIGGVQQDTILAEGLHFRIPWFQYPIIYDIRARPRKISSPTGSKDLQMV NISLRVLSRPNAQELPSMYQRLGLDYEERVLPSIVNEVLKSVVAKFNASQLITQRAQV SLLIRRELTERAKDFSLILDDVAITELSFSREYTAAVEAKQVALSKNPGYIKLRKIRA AQNISKTIATSQNRIYLTADNLVLNLQDESFTRGSDSLIKGKK
tccgtatgcgcgattcctgtgcgcgaagttcgggtccgtagtgggctaagggggagggtttcaaagggagcgcacttccgctgccctttctttcgccagccttacgggcccgaaccctcgtgtgaagggtgcagtacctaagccggagcggggtagaggcgggccggcacccccttctgacctccagtgccgccggcctcaagatcagacatggcccagaacttgaaggacttggcgggacggctgcccgccgggccccggggcatgggcacggccctgaagctgttgctgggggccggcgccgtggcctacggtgtgcgcgaatctgtgttcaccgtggaaggcgggcacagagccatcttcttcaatcggatcggtggagtgcagcaggacactatcctggccgagggccttcacttcaggatcccttggttccagtaccccattatctatgacattcgggccagacctcgaaaaatctcctcccctacaggctccaaagacctacagatggtgaatatctccctgcgagtgttgtctcgacccaatgctcaggagcttcctagcatgtaccagcgcctagggctggactacgaggaacgagtgttgccgtccattgtcaacgaggtgctcaagagtgtggtggccaagttcaatgcctcacagctgatcacccagcgggcccaggtatccctgttgatccgccgggagctgacagagagggccaaggacttcagcctcatcctggatgatgtggccatcacagagctgagctttagccgagagtacacagctgctgtagaagccaaacaagtggcactgagcaagaaccctggctacatcaaacttcgcaagattcgagcagcccagaatatctccaagacgatcgccacatcacagaatcgtatctatctcacagctgacaaccttgtgctgaacctacaggatgaaagtttcaccaggggaagtgacagcctcatcaagggtaagaaatgagcctagtcaccaagaactccacccccagaggaagtggatctgcttctccagtttttgaggagccagccaggggtccagcacagccctaccccgccccagtatcatgcgatggtcccccacaccggttccctgaacccctcttggattaaggaagactgaagactagccccttttctggggaattactttcctcctccctgtgttaactggggctgttggggacagtgcgtgatttctcagtgatttcctacagtgttgttccctccctcaaggctgggaggagataaacaccaacccaggaattctcaataaatttttattacttaacctgaaaaaaaaaaaaaaaa

SEQ ID NO: 30 - protein, 261 aa, Homo sapiens
MAQNLKDLAGRLPAGPRGMGTALKLLLGAGAVAYGVRESVFTVEGGHRAIFFNRIGGVQQDTILAEGLHFRIPWFQYPIIYDIRARPRKISSPTGSKDLQMVNISLRVLSRPNAQELPSMYQRLGLDYEERVLPSIVNEVLKSVVAKFNASQLITQRAQVSLLIRRELTERAKDFSLILDDVAITELSFSREYTAAVEAKQVALSKNPGYIKLRKIRAAQNISKTIATSQNRIYLTADNLVLNLQDESFTRGSDSLIKGKK

SEQ ID NO: 31 - DNA, 14895 bp, Homo sapiens
  source 1..14895: mol_type = unassigned DNA
  CDS 172..6705: protein_id = 32; translation = MEEILRKLQKEASGSKYKAIKESCTWALETLGGLDTIVKIPPHV LREKCLLPLQLALESKNVKLAQHALAGMQKLLSEERFVSMETDSDEKQLLNQILNAVK VTPSLNEDLQVEVMKVLLCITYTPTFDLNGSAVLKIAEVCIETYISSCHQRSINTAVR ATLSQMLSDLTLQLRQRQENTIIENPDVPQDFGNQGSTVESLCDDVVSVLTVLCEKLQ AAINDSQQLQLLYLECILSVLSSSSSSMHLHRRFTDLIWKNLCPALIVILGNPIHDKT ITSAHTSSTSTSLESDSASPGVSDHGRGSGCSCTAPALSGPVARTIYYIAAELVRLVG SVDSMKPVLQSLYHRVLLYPPPQHRVEAIKIMKEILGSPQRLCDLAGPSSTESESRKR SISKRKSHLDLLKLIMDGMTEACIKGGIEACYAAVSCVCTLLGALDELSQGKGLSEGQ VQLLLLRLEELKDGAEWSRDSMEINEADFRWQRRVLSSEHTPWESGNERSLDISISVT TDTGQTTLEGELGQTTPEDHSGNHKNSLKSPAIPEGKETLSKVLETEAVDQPDVVQRS HTVPYPDITNFLSVDCRTRSYGSRYSESNFSVDDQDLSRTEFDSCDQYSMAAEKDSGR SDVSDIGSDNCSLADEEQTPRDCLGHRSLRTAALSLKLLKNQEADQHSARLFIQSLEG LLPRLLSLSNVEEVDTALQNFASTFCSGMMHSPGFDGNSSLSFQMLMNADSLYTAAHC ALLLNLKLSHGDYYRKRPTLAPGVMKDFMKQVQTSGVLMVFSQAWIEELYHQVLDRNM LGEAGYWGSPEDNSLPLITMLTDIDGLESSAIGGQLMASAATESPFAQSRRIDDSTVA GVAFARYILVGCWKNLIDTLSTPLTGRMAGSSKGLAFILGAEGIKEQNQKERDAICMS LDGLRKAARLSCALGVAANCASALAQMAAASCVQEEKEEREAQEPSDAITQVKLKVEQ KLEQIGKVQGVWLHTAHVLCMEAILSVGLEMGSHNPDCWPHVFRVCEYVGTLEHNHFS DGASQPPLTISQPQKATGSAGLLGDPECEGSPPEHSPEQGRSLSTAPVVQPLSIQDLV REGSRGRASDFRGGSLMSGSSAAKVVLTLSTQADRLFEDATDKLNLMALGGFLYQLKK ASQSQLFHSVTDTVDYSLAMPGEVKSTQDRKSALHLFRLGNAMLRIVRSKARPLLHVM RCWSLVAPHLVEAACHKERHVSQKAVSFIHDILTEVLTDWNEPPHFHFNEALFRPFER IMQLELCDEDVQDQVVTSIGELVEVCSTQIQSGWRPLFSALETVHGGNKSEMKEYLVG DYSMGKGQAPVFDVFEAFLNTDNIQVFANAATSYIMCLMKFVKGLGEVDCKEIGDCAP APGAPSTDLCLPALDYLRRCSQLLAKIYKMPLKPIFLSGRLAGLPRRLQEQSASSEDG IESVLSDFDDDTGLIEVWIILLEQLTAAVSNCPRQHQPPTLDLLFELLRDVTKTPGPG FGIYAVVHLLLPVMSVWLRRSHKDHSYWDMASANFKHAIGLSCELVVEHIQSFLHSDI RYESMINTMLKDLFELLVACVAKPTETISRVGCSCIRYVLVTAGPVFTEEMWRLACCA LQDAFSATLKPVKDLLGCFHSGTESFSGEGCQVRVAAPSSSPSAEAEYWRIRAMAQQV FMLDTQCSPKTPNNFDHAQSCQLIIELPPDEKPNGHTKKSVSFREIVVSLLSHQVLLQ NLYDILLEEFVKGPSPGEEKTIQVPEAKLAGFLRYISMQNLAVIFDLLLDSYRTAREF DTSPGLKCLLKKVSGIGGAANLYRQSAMSFNIYFHALVCAVLTNQETITAEQVKKVLF EDDERSTDSSQQCSSEDEDIFEETAQVSPPRGKEKRQWRARMPLLSVQPVSNADWVWL VKRLHKLCMELCNNYIQMHLDLENCMEEPPIFKGDPFFILPSFQSESSTPSTGGFSGK ETPSEDDRSQSREHMGESLSLKAGGGDLLLPPSPKVEKKDPSRKKEWWENAGNKIYTM AADKTISKLMTEYKKRKQQHNLSAFPKEVKVEKKGEPLGPRGQDSPLLQRPQHLMDQG QMRHSFSAGPELLRQDKRPRSGSTGSSLSVSVRDAEAQIQAWTNMVLTVLNQIQILPD QTFTALQPAVFPCISQLTCHVTDIRVRQAVREWLGRVGRVYDIIV
gtggcccgcggcatggagcgggcgtgattcatcagcatccgcgccggggcggcatgggggcgcgcgcggcggccgcctaggcgcccagggccaggcagcggcggcttccccggcccggctcgcccgcgcttctctccctgtgggcggcggcccggcgcctggaaggtcaagatggaagaaatcctgaggaagctgcagaaggaggcgtccgggagcaagtacaaagccatcaaggagagctgcacctgggccctggaaactctaggtggtctggataccattgtcaagatccctccacatgtactgagggagaaatgcctgctgcctctccagttggctttggaatccaagaatgtgaagctggcccaacatgctttggcagggatgcagaagcttctgtcggaagagaggtttgtatccatggaaacagattctgatgagaagcagctgctcaatcagatactgaatgccgtgaaagtgacgccttcgctcaacgaggacctgcaggtggaagtgatgaaggttttactatgcatcacctacacgccaacatttgatctgaatgggagtgccgtgctgaagatcgcggaggtgtgcattgagacgtacataagcagctgtcaccagcgtagcataaacactgctgtgcgggcaactctcagtcaaatgctgagtgacttgactttacagttacgacagaggcaggagaatacgataattgaaaacccagatgtcccacaggatttcgggaatcaagggtcaacagtagagtccctctgtgatgatgttgtctctgtactcaccgtcctgtgtgagaagctgcaagccgccataaatgacagccagcagctgcagcttctctacctggagtgcatcctgtctgtgctcagcagctcctcctcctccatgcacctgcacaggcgcttcacggacctgatctggaaaaacctctgccctgctctcatcgtgatcttggggaatccaattcatgacaaaaccatcacctctgctcacaccagcagcaccagtaccagcctggagtcggactctgcgtctccgggagtgtctgaccacggccgaggatcaggctgctcctgcactgcgccggccctgagcggacctgtggctcggactatctattacatcgcagccgagctggtccggctggtggggtctgtggactccatgaagcccgtgctccagtccctctaccaccgagtgctgctctaccccccaccccagcaccgggtggaagccatcaaaataatgaaagagatacttgggagcccacagcgtctctgtgacttggcaggacccagctccactgaatcagagtccagaaaaagatcaatttcaaaaagaaagtctcatctggatctcctcaaactcatcatggatggcatgaccgaagcatgcatcaagggtggcatcgaagcttgctatgcagccgtgtcctgtgtctgcaccttgctgggtgccctggatgagctcagccaggggaagggcttgagcgaaggtcaggtgcaactgctgcttctgcgccttgaggagctgaaggatggggctgagtggagccgagattccatggagatcaatgaggctgacttccgctggcagcggcgagtgctgtcctcagaacacacgccgtgggagtcagggaacgagaggagccttgacatcagcatcagtgtcaccacagacacaggccagaccactctcgagggagagttgggtcagactacacccgaggaccattcgggaaaccacaagaacagtctcaagtcgccagccatcccagagggtaaggagacgctgagcaaagtattggaaacagaggcggtagaccagccagatgtcgtgcagagaagccacacggtcccttaccctgacataactaacttcctgtcagtagactgcaggacaaggtcctatggatctaggtatagtgagagcaattttagcgttgatgaccaagacctttctaggacagagtttgattcctgtgatcagtactctatggcagcagaaaaggactcgggcaggtccgacgtgtcagacattgggtcggacaactgttcactagccgatgaagagcagacaccccgggactgcctaggccaccggtccctgcgaactgccgccctgtctctaaaactgctgaagaaccaggaggcggatcagcacagcgccaggctgttcatacagtccctggaaggcctcctccctcggctcctgtctctctccaatgtagaggaggtggacaccgctctgcagaactttgcctctactttctgctcaggcatgatgcactctcctggctttgacgggaatagcagcctcagcttccagatgctgatgaacgcagacagcctctacacagctgcacactgcgccctgctcctcaacctgaagctctcccacggtgactactacaggaagcggccgaccctggcgccaggcgtgatgaaggacttcatgaagcaggtgcagaccagcggcgtgctgatggtcttctctcaggcctggattgaggagctctaccatcaggtgctcgacaggaacatgcttggagaggctggctattggggcagcccagaagataacagccttcccctcatcacaatgctgaccgatattgacggcttagagagcagtgccattggtggccagctgatggcctcggctgctacagagtctcctttcgcccagagcaggagaattgatgactccacagtggcaggcgtggcatttgctcgctatattctggtgggctgctggaagaacttgatcgatactttatcaaccccactgactggtcgaatggcggggagctccaaagggctggccttcattctgggagctgaaggcatcaaagagcagaaccagaaggagcgggacgccatctgcatgagcctcgacgggctgcggaaagccgcacggctgagctgcgctctaggcgttgctgctaactgcgcctcagcccttgcccagatggcagctgcctcctgtgtccaagaagaaaaagaagagagggaggcccaagaacccagtgatgccatcacacaagtgaaactaaaagtggagcagaaactggagcagattgggaaggtgcagggggtgtggctgcacactgcccacgtcttgtgcatggaggccatcctcagcgtaggcctggagatgggaagccacaacccggactgctggccacacgtgttcagggtgtgtgaatacgtgggcaccctggagcacaaccacttcagcgatggtgcctcgcagccccctctgaccatcagccagccccagaaggccactggaagcgctggcctccttggggaccccgagtgtgagggctcgccccccgagcacagcccggagcaggggcgctccctgagcacggcccctgtcgtccagcccctgtccatccaggacctcgtccgggaaggcagccggggtcgggcctccgacttccgcggcgggagcctcatgagcgggagcagcgcggccaaggtggtgctcaccctctccacgcaagccgacaggctctttgaagatgctacggataagttgaacctcatggccttgggaggttttctttaccagctgaagaaagcatcgcagtctcagcttttccattctgttacagatacagttgattactctctggcaatgccaggagaagttaaatccactcaagaccgaaaaagcgccctccacctgttccgcctggggaatgccatgctgaggattgtgcggagcaaagcacggcccctgctccacgtgatgcgctgctggagccttgtggccccacacctggtggaggctgcttgccataaggaaagacatgtgtctcagaaggctgtttccttcatccatgacatactgacagaagtcctcactgactggaatgagccacctcattttcacttcaatgaagcactcttccgacctttcgagcgcattatgcagctggaattgtgtgatgaggacgtccaagaccaggttgtcacatccattggtgagctggttgaagtgtgttccacgcagatccagtcgggatggagacccttgttcagtgccctggaaacagtgcatggcgggaacaagtcagagatgaaggagtacctggttggtgactactccatgggaaaaggccaagctccagtgtttgatgtatttgaagcttttctcaatactgacaacatccaggtctttgctaatgcagccactagctacatcatgtgccttatgaagtttgtcaaaggactgggggaggtggactgtaaagagattggagactgtgccccagcacccggagccccgtccacagacctgtgcctcccggccctggattacctcaggcgctgctctcagttattggccaaaatctacaaaatgcccttgaagccaatattccttagtgggagacttgccggcttgcctcgaagacttcaggaacagtcagccagcagtgaggatggaattgaatcagtcctgtctgattttgatgatgacaccggtctgatagaagtctggataatcctgctggagcagctgacagcggctgtgtccaattgtccacggcagcaccaaccaccaactctggatttactctttgagctgttgagagatgtgacgaaaacaccaggaccagggtttggtatctatgcagtggttcacctcctccttcctgtgatgtccgtttggctccgccggagccataaagaccattcctactgggatatggcctctgccaatttcaagcacgctattggtctgtcctgtgagctggtggtggagcacattcaaagctttctacattcagatatcaggtacgagagcatgatcaataccatgctgaaggacctctttgagttgctggtcgcctgtgtggccaagcccactgaaaccatctccagagtgggctgctcctgtattagatacgtccttgtgacagcgggccctgtgttcactgaggagatgtggaggcttgcctgctgtgccctgcaagatgcgttctctgccacactcaagccagtgaaggacctgctgggctgcttccacagcggcacggagagcttcagcggggaaggctgccaggtgcgagtggcggccccgtcctcctccccaagtgccgaggccgagtactggcgcatccgagccatggcccagcaggtgtttatgctggacacccagtgctcaccaaagacaccaaacaactttgaccacgctcagtcctgccagctcattattgagctgcctcctgatgaaaaaccaaatggacacaccaagaaaagcgtgtctttcagggaaattgtggtgagcctgctgtctcatcaggtgttactccagaacttatatgacatcttgttagaagagtttgtcaaaggcccctctcctggagaggaaaagacgatacaagtgccagaagccaagctggctggcttcctcagatacatctctatgcagaacttggcagtcatattcgacctgctgctggactcttataggactgccagggagtttgacaccagccccgggctgaagtgcctgctgaagaaagtgtctggcatcgggggcgccgccaacctctaccgccagtctgcgatgagctttaacatttatttccacgccctggtgtgtgctgttctcaccaatcaagaaaccatcacggccgagcaagtgaagaaggtcctttttgaggacgacgagagaagcacggattcttcccagcagtgttcatctgaggatgaagacatctttgaggaaaccgcccaggtcagccccccgagaggcaaggagaagagacagtggcgggcacggatgcccttgctcagcgtccagcctgtcagcaacgcagattgggtgtggctggtcaagaggctgcacaagctgtgcatggaactgtgcaacaactacatccagatgcacttggacctggagaactgtatggaggagcctcccatcttcaagggcgacccgttcttcatcctgccctccttccagtccgagtcatccaccccatccaccgggggcttctctgggaaagaaaccccttccgaggatgacagaagccagtcccgggagcacatgggcgagtccctgagcctgaaggccggtggtggggacctgctgctgccccccagccccaaagtggagaagaaggatcccagccggaagaaggagtggtgggagaatgcggggaacaaaatctacaccatggcagccgacaagaccatttcaaagttgatgaccgaatacaaaaagaggaaacagcagcacaacctgtccgcgttccccaaagaggtcaaagtggagaagaaaggagagccactgggtcccaggggccaggactccccgctgcttcagcgtccccagcacttgatggaccaagggcaaatgcggcattccttcagcgcaggccccgagctgctgcgacaggacaagaggccccgctcaggctccaccgggagctccctcagtgtctcggtgagagacgcagaagcacagatccaggcatggaccaacatggtgctaacagttctcaatcagattcagattctcccagaccagaccttcacggccctccagcccgcagtgttcccgtgcatcagtcagctgacctgtcacgtgaccgacatcagagttcgccaggctgtgagggagtggctgggcagggtgggccgtgtctatgacatcattgtgtagccgactcctgttctactctcccaccaaataacagtagtgagggttagagtcctgccaatacagctgttgcattttccccaccactagccccacttaaactactactactgtctcagagaacagtgtttcctaatgtaaaaagcctttccaaccactgatcagcattggggccatactaaggtttgtatctagatgacacaaacgatattctgattttgcacattattatagaagaatctataatccttgatatgtttctaactcttgaagtatatttcccagtgcttttgcttacagtgttgtccccaaatgggtcattttcaaggattactcatttgaaaacactatattgatccatttgatccatcatttaaaaaataaatacaattcctaaggcaatatctgctggtaagtcaagctgataaacactcagacatctagtaccagggattattaattggaggaagatttatggttatgggtctggctgggaagaagacaactataaatacatattcttgggtgtcataatcaagaaagaggtgacttctgttgtaaaataatccagaacacttcaaaattattcctaaatcattaagattttcaggtattcaccaatttccccatgtaaggtactgtgttgtacctttatttctgtatttctaaaagaagaaagttctttcctagcagggtttgaagtctgtggcttatcagcctgtgacacagagtacccagtgaaagtggctggtacgtagattgtcaagagacataagaccgaccagccaccctggctgttcttgtggtgtttgtttccatccccaaggcaaacaaggaaaggaaaggaaagaagaaaaggtgccttagtcctttgttgcacttccatttccatgccccacaattgtctgaacataaggtatagcatttggtttttaagaaaacaaaacattaagacgcaactcattttatatcaacacgcttggaggaaagggactcagggaagggagcagggagtgtggggtggggatggattatgatgaaatcattttcaatcttaaaatataatacaacaatcttgcaaaattatggtgtcagttacacaagctctagtctcaaaatgaaagtaatggagaaagacactgaaatttagaaaattttgtcgatttaaaatatttctcctatctaccaagtaaagttaccctatgtttgatgtctttgcattcagaccaatatttcaggtggatatttctaagtattactagaaaatacgtttgaaagctttatcttattatttacagtatttttatatttcttacattatcctaatgattgaaaactcctcaatcaagcttacttacacacattctacagagttatttaaggcatacattataatctcccagccccattcataatgaataagtcaccctttaaatataagacacaaattctacagtattgaaataaggatttaaaggggtatttgtaaactttgccctccttgagaaatatggaactaccttagaggttaagaggaaggcagtgttctgacttctttaggtgatctgaaaaaaacacccttatcatccagtgtaccatctagagatcaccacagaatccatttttttcccagttccacaaaacactctgtttgccttcagtttttactcactagacaataattcaagtttagaaacaggtaatcagctatttgatcttaaaaggcaatgaattgttgggatatcagtgaactatgttgtatacttttgaatttttacattttataaatggaattgaaagttggataactgctttttttaaattttccaacagaagtaacaccacagttgctttgtttctttttatagcttacctgaggttcagttcttctttgtgaacctgtgagtactccacagtttactgggggaaaaggcttcagtaaagcagaggctagaattacagtatttatacatagcaacttttcataaagtagaaaaattcaaaggaagctgtctcaatttgagaataccagctgggcacggtggctcacgcctgtaatcccagcacttactttgggaggccaaggtgggcagataacctgcggtcaggagtttgagaccaggctggacaacatggtgaaacctcgtctctactaaaaatacaaaaattagccaggtgtggtaggatgcacctgtaatcccagctacttaggaggccgagacaggagaatcgctcgaacccaggaggcggacgttgcagtgagccaagattgcaccattgcactccagactgggtgacaagagtgaaactccatctaaaaaaaaaaaaaaaaaaaagtgaatactgtatcccaaagtatgttagttgtttgtttggaaatcagcattctccccgatgctctattatgggatccaaaattcttgaacataagtttaccctgtactgtgtccaaacactgttctagttctagcctgattatgggtcccaagaataaaaggatgagtaggtgtacagagctcttgacctacaattttttaagagtgttttggtaccttcccattgtcttctctataactcagtcctaacatactctgcactcgagttaccagccatccacactgacatcagatttcaaccagaaccatcactgagtgacagcagtacttctcagaggtatttgcagcttgatgcaaagtagtctctaatgagtaggcattcaggtggttcttcccagcaggtggagaagaaagggaggagatgaagaacactgagaggggagtggcaccttcccaggctgcccagctcagtctcttgccctgttcctgtgactcagctgcccactcccccaactttgtttccctccctcccagtctctgaaagtgtcaggtgtttctctcctcacagtctcttttgcagcaacagtaagacaaaattcaaggcagccttttaaagttacgaacagttattagcatgtatttacagacctaagcagaatgagagtttatacattgtttttagttgcctgtatttatagccaaaagtatattaccttaaagttgagatctttctcttcttttcctaaattttggtaaagtgtgcttcatgaaacaaacatctggaaaactccaagtataagagaccctggactgatgatggcccagccaagtatatggagggacagagttctctctgtcattaatgaggacatcggttttcacaattgaacctcatgcactgtccacagcatctcacctagctcctgtatctcctgatctgcttttaaaaatagttagttaggctgcctttttacaccaccttctctctctccccttgtggtaattttccagccttccccatagatataaaactagaacacctttatgatttggggtctatgtaatgactgaccgataagaacccaggcagatgctaacatacttaacagctcgcattaaaatactttaaatcaggcgtgatggctcattcctgtaatctcaagcactttgggaggctaaggtgggtggatctcttgaggtcaggagttcgagaccaacctggccaacgtggtgaaaccccgtctctactaaaaatacaaaattagccgggcatggtggcagctgcctgtaatcccagctactcgggaagctgaggcaggagaattgcttgaacctgggaggtggggattgcagtcagccaagattgttctgcagcatgggtgacaaagtgagacttcgtctcaagtaaataaaactaaaatttttaaatcaaacatgacaaaaatgttaatataattcagaagtaccttgaaattgaaacatatttgtgcaatgatcattaggctttttgtccttgttgttttaaaatgaggcttatacagagtgagttgagagtcaagtagccttcgctgtgagacggtaatgcagttatataatagatacccttgactttgccagattcatcacaatactgcttatacaggaaagttttctcagaaaggaaaatccattagtatcagtcccatcaagccaaacagaatgaagacctttgatagtaatagcaagaggttacaaatagcagggaggaggcgagtagtgaatgtcactgtgattgcaaacccttacctgtattatcacacgtagtcctcacaacaaccttgtgagacaagtgttgtgttcctcattttttcagaggggaacacagacccagagaggttaagaaatttgcccaagataacaagtaaaaggcaaagttggttgcaaaagaggtgtttctgaattcaagggccatactctctctctgacaacatgctctaagtccatagagtaagcactctagtatgaaaaaaagtttcaaggaacgaggccatgaaaatgagactatttgacatctcagatctgtctgggatgttatggaggtttttaaaaataaagttgaaaaaagaaaatgaatcatgtttatacataaaaaaatcacatgtaacacatttcaagtgtttgaaaataaaaccaaaatctaaactttagtcttcaagcagacattcagtgttactttagaaaactcactgaattaggtggaaatgatggaataatactattcatggccagctattaacacagaagaacatggcagtgtgtgtctggaacggcatgcacaatttgtaaacctttttcaaatatcatttaatcaactcagaataaagtgccctgtagccaacagtgcctctttacttgcttctctgggaaatacatggtactaaattagtagcacaaagtttgggaatatgcaaaataatggataaccatttttcaaaatgtacattctctgaagaggaagcagctggttggacaggatttcttgaagagccaggtgctaagggcatcaggtcgacatccatagtaaccatgtgccataacatctacacatttccacttgttttacagacaaggtaacaggcagaaggaaaatccagagtcttgcagtaagcagatgacaaaacttcaatatgcttgggcaccacttaggtgaccccagggagatttagtgtggccttaggaaagcaaaagagcactttttattggaaatatgagcttgtcactgggaaagatttgtaaaattgatcaagaacttgatttataattatgcctcaaaaaaaaaagttctcatttagtagtggagcaatctagaaaacataccttttttgtttgtttggaagatcctctttccctggctgtattgtagtgtttgctatttgatgtggaaataactaataacttaagattttggaacagaacaccctttagatttccaaaacacaattcttatttcagggaagacagaccaaaaatatctcctgagatcattggtttctttataaattgtggtaccactccatcattgaagagaaaccactaccacaccactagcaccatacagaaccttttctctgtatctttgtacaatactacaaaggggtaccagggaggagagagtggctgaccactttagtgacaaaacagcactccactgctggtgaatcccatctaattatggtccttccacccttttcaaccaccaacaactgttcgtactgttaattcctatcctgaaggtttaaccagtggttgtctagtatcttctgtctttagaacagtggttctcaaactttagtacacatcagcatcacctggagggcctttttttaaaataagacacagattgctgggctcatggtcagagttcccagttaagtaaatcaggaaatttgtatttctaacaagtttataggtgaggccaatactgctgttttgggaactatgctttgagaaccactgccttgaaaaaatttccaacttctacctttaagatcagcctgacttatcaaacgctagagaaaaactgaatctacccttgggcagatgacttgggattggattctatacagcagtcttgctcaatcttcccagtttccagttttattataccaacaattggtttttacaagctagaagacaatgaatgtataagttctatggaacagtgagataaatctaagcttcttgtctttgtatttagaaacattgattctatggatgatcatttgtatcatgttgaccctttgacttgtactgaaggtgattttaaatttaagtatgtagtgtttgaatttcttccatccatgtcgttttaatgagatgtttccatgtcagctcctttacagccttggctcctggcttacagatttttgaatagttgtttgcttgccagttgttttacatctttcattggccaccaaaatattagccatttgagatgagatgagactacttgttgtaccttcatctttcatttaattttctggcgtaaattaacattttaatttcatatatatctgtaaagagtctacccaaaggcttcacggaaatttgcaaaatgaactaattcccttttaagcagcaggtgtgcctgtttttgacttttcagtaaatatgttgtttgtgcacatatctacatggtggagaccatattcattatttcatcttccaaataatgggaaaaatataaaagtgaatcagtgtgctttgggaattcagtgaaatcatgttaactcatatagagggggccttagtttatctcttctttactgaattaattagttttggaaattcttttaccattaaaaaaaattaaggaccatacagagaatgatttaagaaaaaacaagtcacttaaaaatcatcacctatttataaactgtattaattacacataatgcttattgattcaatgaggtttctctaaagacttctgcttaataaatatgctgacttcatttaaattagtttagactattgtaggaatggaaggaaatgattatatttactagaattagtgagatcagaaagcatatcagaatgttgatgatatcaaggagacaatctacagagtttttgcctctgtggatggaaataagggtgtttttttttggttttttttttactttagtttcccataatttttggaaattatgtgtgcatttagttcttttagtaacactgattttaaaattaaatttcaaaagtcaatctctaagagtaatttatttttgttttaccaaccagtgccaaaaaggagaggagggaatccaaaagccaatcttttgaaccaatgtgtaaaagattatgttttttcttaaagttagggaggctcgggccctgacactgccagccccagtgagcatccctggctacctcgggattatgtgcaagctgctttgtcctacatttctttcatctggttcttattgggagtgcttctctctaataaaaattgatttcccacaaaataggcaaagctgaacaaagatgaatgcttttgataagttgggtttcacttcagttgaaacaatgtgatagaatatccaggtgtggcatgatggggcaggaggaggtgcctagagggaaaagttatttttgtttcttagtgttgtgttgtggggatgggacagataagaataagatgtttattgccctaatcatgctaagagactattattcaatatgcttttcccgcttttctaagaggaataaacttagacaaattacattataaacagttcccctactactatctcccactctagataaagccagtgggtggtatgggtccttttattccttatagtattatgccaaagaatcaacttattttcattgaagattataaataaatgaagcttgttatagccataatgatttgagtcagtataccattttacctataaaatgcaaaattcatccttgcaaccccattcaccaggagccttgaagcattttgtttactccaaaggccttgtcaaggaagcataattttttgttttgccttcttatttagtcagtttggtcatatttacttaaaaaaacaaactgaaaatcacactcctttatatgttgatataactgattttatagaatctgtctgttctttgtttaacaggtctctgtaagcaagcttgcaagtgtattttgtgtacattttatctgaggtggaaatgaaaattctaaagagaaaatattttaaaagatattgtatttatgttgcttgtgttgtagaataaagattcaaatgcattaaaaatctggtacatgaaacaattgtgtttactgaataaatatatataaataaaaaaaaaaaaaa

SEQ ID NO: 32 - protein, 2177 aa, Homo sapiens
MEEILRKLQKEASGSKYKAIKESCTWALETLGGLDTIVKIPPHVLREKCLLPLQLALESKNVKLAQHALAGMQKLLSEERFVSMETDSDEKQLLNQILNAVKVTPSLNEDLQVEVMKVLLCITYTPTFDLNGSAVLKIAEVCIETYISSCHQRSINTAVRATLSQMLSDLTLQLRQRQENTIIENPDVPQDFGNQGSTVESLCDDVVSVLTVLCEKLQAAINDSQQLQLLYLECILSVLSSSSSSMHLHRRFTDLIWKNLCPALIVILGNPIHDKTITSAHTSSTSTSLESDSASPGVSDHGRGSGCSCTAPALSGPVARTIYYIAAELVRLVGSVDSMKPVLQSLYHRVLLYPPPQHRVEAIKIMKEILGSPQRLCDLAGPSSTESESRKRSISKRKSHLDLLKLIMDGMTEACIKGGIEACYAAVSCVCTLLGALDELSQGKGLSEGQVQLLLLRLEELKDGAEWSRDSMEINEADFRWQRRVLSSEHTPWESGNERSLDISISVTTDTGQTTLEGELGQTTPEDHSGNHKNSLKSPAIPEGKETLSKVLETEAVDQPDVVQRSHTVPYPDITNFLSVDCRTRSYGSRYSESNFSVDDQDLSRTEFDSCDQYSMAAEKDSGRSDVSDIGSDNCSLADEEQTPRDCLGHRSLRTAALSLKLLKNQEADQHSARLFIQSLEGLLPRLLSLSNVEEVDTALQNFASTFCSGMMHSPGFDGNSSLSFQMLMNADSLYTAAHCALLLNLKLSHGDYYRKRPTLAPGVMKDFMKQVQTSGVLMVFSQAWIEELYHQVLDRNMLGEAGYWGSPEDNSLPLITMLTDIDGLESSAIGGQLMASAATESPFAQSRRIDDSTVAGVAFARYILVGCWKNLIDTLSTPLTGRMAGSSKGLAFILGAEGIKEQNQKERDAICMSLDGLRKAARLSCALGVAANCASALAQMAAASCVQEEKEEREAQEPSDAITQVKLKVEQKLEQIGKVQGVWLHTAHVLCMEAILSVGLEMGSHNPDCWPHVFRVCEYVGTLEHNHFSDGASQPPLTISQPQKATGSAGLLGDPECEGSPPEHSPEQGRSLSTAPVVQPLSIQDLVREGSRGRASDFRGGSLMSGSSAAKVVLTLSTQADRLFEDATDKLNLMALGGFLYQLKKASQSQLFHSVTDTVDYSLAMPGEVKSTQDRKSALHLFRLGNAMLRIVRSKARPLLHVMRCWSLVAPHLVEAACHKERHVSQKAVSFIHDILTEVLTDWNEPPHFHFNEALFRPFERIMQLELCDEDVQDQVVTSIGELVEVCSTQIQSGWRPLFSALETVHGGNKSEMKEYLVGDYSMGKGQAPVFDVFEAFLNTDNIQVFANAATSYIMCLMKFVKGLGEVDCKEIGDCAPAPGAPSTDLCLPALDYLRRCSQLLAKIYKMPLKPIFLSGRLAGLPRRLQEQSASSEDGIESVLSDFDDDTGLIEVWIILLEQLTAAVSNCPRQHQPPTLDLLFELLRDVTKTPGPGFGIYAVVHLLLPVMSVWLRRSHKDHSYWDMASANFKHAIGLSCELVVEHIQSFLHSDIRYESMINTMLKDLFELLVACVAKPTETISRVGCSCIRYVLVTAGPVFTEEMWRLACCALQDAFSATLKPVKDLLGCFHSGTESFSGEGCQVRVAAPSSSPSAEAEYWRIRAMAQQVFMLDTQCSPKTPNNFDHAQSCQLIIELPPDEKPNGHTKKSVSFREIVVSLLSHQVLLQNLYDILLEEFVKGPSPGEEKTIQVPEAKLAGFLRYISMQNLAVIFDLLLDSYRTAREFDTSPGLKCLLKKVSGIGGAANLYRQSAMSFNIYFHALVCAVLTNQETITAEQVKKVLFEDDERSTDSSQQCSSEDEDIFEETAQVSPPRGKEKRQWRARMPLLSVQPVSNADWVWLVKRLHKLCMELCNNYIQMHLDLENCMEEPPIFKGDPFFILPSFQSESSTPSTGGFSGKETPSEDDRSQSREHMGESLSLKAGGGDLLLPPSPKVEKKDPSRKKEWWENAGNKIYTMAADKTISKLMTEYKKRKQQHNLSAFPKEVKVEKKGEPLGPRGQDSPLLQRPQHLMDQGQMRHSFSAGPELLRQDKRPRSGSTGSSLSVSVRDAEAQIQAWTNMVLTVLNQIQILPDQTFTALQPAVFPCISQLTCHVTDIRVRQAVREWLGRVGRVYDIIV

SEQ ID NO: 33 - protein, 13 aa, synthetic construct
  REGION 1..13: note = PHB2-binding peptide derived from BIG3
QMLSDLTLQLRQR

SEQ ID NO: 34 - protein, 12 aa, synthetic construct
  REGION 1..12: note = PHB2 peptide No. 34
MAQNLKDLAGRL

SEQ ID NO: 35 - protein, 12 aa, synthetic construct
  REGION 1..12: note = PHB2 peptide No. 35
QNLKDLAGRLPA

SEQ ID NO: 36 - protein, 12 aa, synthetic construct
  REGION 1..12: note = PHB2 peptide No. 36
LKDLAGRLPAGP

SEQ ID NO: 37 - protein, 12 aa, synthetic construct
  REGION 1..12: note = PHB2 peptide No. 37
DLAGRLPAGPRG

SEQ ID NO: 38 - protein, 12 aa, synthetic construct
  REGION 1..12: note = PHB2 peptide No. 38
AGRLPAGPRGMG

SEQ ID NO: 39 - protein, 12 aa, synthetic construct
  REGION 1..12: note = PHB2 peptide No. 39
PAGPRGMGTALK

SEQ ID NO: 40 - protein, 12 aa, synthetic construct
  REGION 1..12: note = PHB2 peptide No. 40
GPRGMGTALKLL

SEQ ID NO: 41 - protein, 12 aa, synthetic construct
  REGION 1..12: note = PHB2 peptide No. 41
RGMGTALKLLLG

SEQ ID NO: 42 - protein, 12 aa, synthetic construct
  REGION 1..12: note = PHB2 peptide No. 42
MGTALKLLLGAG

SEQ ID NO: 43 - protein, 12 aa, synthetic construct
  REGION 1..12: note = PHB2 peptide No. 43
TALKLLLGAGAV

SEQ ID NO: 44 - protein, 15 aa, synthetic construct
  REGION 1..15: note = PHB2 peptide No. 44
ILAEGLHFRIPWFQY

SEQ ID NO: 45 - protein, 15 aa, synthetic construct
  REGION 1..15: note = PHB2 peptide No. 45
AEGLHFRIPWFQYPI

SEQ ID NO: 46 - protein, 15 aa, synthetic construct
  REGION 1..15: note = PHB2 peptide No. 46
GLHFRIPWFQYPIIY

SEQ ID NO: 47 - protein, 14 aa, synthetic construct
  REGION 1..14: note = PHB2 peptide No. 47
HFRIPWFQYPIIYD

SEQ ID NO: 48 - protein, 15 aa, synthetic construct
  REGION 1..15: note = PHB2 peptide No. 48
RIPWFQYPIIYDIRA

SEQ ID NO: 49 - protein, 15 aa, synthetic construct
  REGION 1..15: note = PHB2 peptide No. 49
PWFQYPIIYDIRARP

SEQ ID NO: 50 - protein, 15 aa, synthetic construct
  REGION 1..15: note = PHB2 peptide No. 50
FQYPIIYDIRARPRK

SEQ ID NO: 51 - protein, 15 aa, synthetic construct
  REGION 1..15: note = PHB2 peptide No. 51
YPIIYDIRARPRKIS

SEQ ID NO: 52 - protein, 15 aa, synthetic construct
  REGION 1..15: note = PHB2 peptide No. 52
IIYDIRARPRKISSP

SEQ ID NO: 53 - protein, 15 aa, synthetic construct
  REGION 1..15: note = PHB2 peptide No. 53
YDIRARPRKISSPTG

SEQ ID NO: 54 - protein, 15 aa, synthetic construct
  REGION 1..15: note = PHB2 peptide No. 54
IRARPRKISSPTGSK

SEQ ID NO: 55 - protein, 15 aa, synthetic construct
  REGION 1..15: note = PHB2 peptide No. 55
ARPRKISSPTGSKDL

SEQ ID NO: 56 - protein, 12 aa, synthetic construct
  REGION 1..12: note = PHB2 peptide No. 56
ALPAGPRGMGTA

SEQ ID NO: 57 - protein, 12 aa, synthetic construct
  REGION 1..12: note = PHB2 peptide No. 57
RAPAGPRGMGTA

SEQ ID NO: 58 - protein, 12 aa, synthetic construct
  REGION 1..12: note = PHB2 peptide No. 58
RLAAGPRGMGTA

SEQ ID NO: 59 - protein, 12 aa, synthetic construct
  REGION 1..12: note = PHB2 peptide No. 59
RLPAAPRGMGTA

SEQ ID NO: 60 - protein, 12 aa, synthetic construct
  REGION 1..12: note = PHB2 peptide No. 60
RLPAGARGMGTA

SEQ ID NO: 61 - protein, 12 aa, synthetic construct
  REGION 1..12: note = PHB2 peptide No. 61
RLPAGPAGMGTA

SEQ ID NO: 62 - protein, 12 aa, synthetic construct
  REGION 1..12: note = PHB2 peptide No. 62
RLPAGPRAMGTA

SEQ ID NO: 63 - protein, 12 aa, synthetic construct
  REGION 1..12: note = PHB2 peptide No. 63
RLPAGPRGAGTA

SEQ ID NO: 64 - protein, 12 aa, synthetic construct
  REGION 1..12: note = PHB2 peptide No. 64
RLPAGPRGMATA

SEQ ID NO: 65 - protein, 12 aa, synthetic construct
  REGION 1..12: note = PHB2 peptide No. 65
RLPAGPRGMGAA

SEQ ID NO: 66 - protein, 12 aa, synthetic construct
  REGION 1..12: note = PHB2 peptide No. 66
ALPAGPAGMGTA

SEQ ID NO: 67 - protein, 15 aa, synthetic construct
  REGION 1..15: note = PHB2 peptide No. 67
AYPIIYDIRARPRKI

SEQ ID NO: 68 - protein, 15 aa, synthetic construct
  REGION 1..15: note = PHB2 peptide No. 68
QAPIIYDIRARPRKI

SEQ ID NO: 69 - protein, 15 aa, synthetic construct
  REGION 1..15: note = PHB2 peptide No. 69
QYAIIYDIRARPRKI

SEQ ID NO: 70 - protein, 15 aa, synthetic construct
  REGION 1..15: note = PHB2 peptide No. 70
QYPAIYDIRARPRKI

SEQ ID NO: 71 - protein, 15 aa, synthetic construct
  REGION 1..15: note = PHB2 peptide No. 71
QYPIAYDIRARPRKI

SEQ ID NO: 72 - protein, 15 aa, synthetic construct
  REGION 1..15: note = PHB2 peptide No. 72
QYPIIADIRARPRKI

SEQ ID NO: 73 - protein, 15 aa, synthetic construct
  REGION 1..15: note = PHB2 peptide No. 73
QYPIIYAIRARPRKI

SEQ ID NO: 74 - protein, 15 aa, synthetic construct
  REGION 1..15: note = PHB2 peptide No. 74
QYPIIYDARARPRKI

SEQ ID NO: 75 - protein, 15 aa, synthetic construct
  REGION 1..15: note = PHB2 peptide No. 75
QYPIIYDIAARPRKI

SEQ ID NO: 76 - protein, 15 aa, synthetic construct
  REGION 1..15: note = PHB2 peptide No. 76
QYPIIYDIRAAPRKI

SEQ ID NO: 77 - protein, 15 aa, synthetic construct
  REGION 1..15: note = PHB2 peptide No. 77
QYPIIYDIRARARKI

SEQ ID NO: 78 - protein, 15 aa, synthetic construct
  REGION 1..15: note = PHB2 peptide No. 78
QYPIIYDIRARPAKI

SEQ ID NO: 79 - protein, 15 aa, synthetic construct
  REGION 1..15: note = PHB2 peptide No. 79
QYPIIYDIRARPRAI

SEQ ID NO: 80 - protein, 15 aa, synthetic construct
  REGION 1..15: note = PHB2 peptide No. 80
QYPIIYDIRARPRKA

SEQ ID NO: 81 - protein, 15 aa, synthetic construct
  REGION 1..15: note = PHB2 peptide No. 81
QAPIIYDIAARPRKI

SEQ ID NO: 82 - protein, 14 aa, synthetic construct
  REGION 1..14: note = PHB2 peptide No. 82
EGGHRAIFFNRIGG

SEQ ID NO: 83 - protein, 20 aa, synthetic construct
  REGION 1..20: note = PHB2 peptide No. 83
ESVFTVEGGHRAIFFNRIGG

SEQ ID NO: 84 - protein, 11 aa, synthetic construct
  REGION 1..11: note = PHB2 derived peptide 11-21aa
RLPAGPRGMGT

SEQ ID NO: 85 - protein, 13 aa, synthetic construct
  REGION 1..13: note = PHB2 derived peptide 76-88aa
QYPIIYDIRARPR

SEQ ID NO: 86 - DNA, 6330 bp, Homo sapiens
  source 1..6330: mol_type = unassigned DNA
  CDS 235..2022: protein_id = 87
aggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatactgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaactaaa

SEQ ID NO: 87 - protein, 595 aa, Homo sapiens [sequence omitted]

SEQ ID NO: 88 - DNA, 2169 bp, Homo sapiens
  source 1..2169: mol_type = unassigned DNA
  CDS 469..2061: protein_id = 89; translation = MDIKNSPSSLNSPSSYNCSQSILPLEHGSIYIPSSYVDSHHEYP AMTFYSPAVMNYSIPSNVTNLEGGPGRQTTSPNVLWPTPGHLSPLVVHRQLSHLYAEP QKSPWCEARSLEHTLPVNRETLKRKVSGNRCASPVTGPGSKRDAHFCAVCSDYASGYH YGVWSCEGCKAFFKRSIQGHNDYICPATNQCTIDKNRRKSCQACRLRKCYEVGMVKCG SRRERCGYRLVRRQRSADEQLHCAGKAKRSGGHAPRVRELLLDALSPEQLVLTLLEAE PPHVLISRPSAPFTEASMMMSLTKLADKELVHMISWAKKIPGFVELSLFDQVRLLESC WMEVLMMGLMWRSIDHPGKLIFAPDLVLDRDEGKCVEGILEIFDMLLATTSRFRELKL QHKEYLCVKAMILLNSSMYPLVTATQDADSSRKLAHLLNAVTDALVWVIAKSGISSQQ QSMRLANLLMLLSHVRHASNKGMEHLLNMKCKNVVPVYDLLLEMLNAHVLRGCKSSIT GSECSPAEDSKSKEGSQNPQSQ
ctcggtctttaaaaggaagaaggggcttatcgttaagtcgcttgtgatcttttcagtttctccagctgctggctttttggacacccactcccccgccaggaggcagttgcaagcgcggaggctgcgagaaataactgcctcttgaaacttgcagggcgaagagcaggcggcgagcgctgggccggggagggaccacccgagctgcgacgggctctggggctgcggggcagggctggcgcccggagcctgagctgcaggaggtgcgctcgctttcctcaacaggtggcggcggggcgcgcgccgggagaccccccctaatgcgggaaaagcacgtgtccgcattttagagaaggcaaggccggtgtgtttatctgcaagccattatacttgcccacgaatctttgagaacattataatgacctttgtgcctcttcttgcaaggtgttttctcagctgttatctcaagacatggatataaaaaactcaccatctagccttaattctccttcctcctacaactgcagtcaatccatcttacccctggagcacggctccatatacataccttcctcctatgtagacagccaccatgaatatccagccatgacattctatagccctgctgtgatgaattacagcattcccagcaatgtcactaacttggaaggtgggcctggtcggcagaccacaagcccaaatgtgttgtggccaacacctgggcacctttctcctttagtggtccatcgccagttatcacatctgtatgcggaacctcaaaagagtccctggtgtgaagcaagatcgctagaacacaccttacctgtaaacagagagacactgaaaaggaaggttagtgggaaccgttgcgccagccctgttactggtccaggttcaaagagggatgctcacttctgcgctgtctgcagcgattacgcatcgggatatcactatggagtctggtcgtgtgaaggatgtaaggccttttttaaaagaagcattcaaggacataatgattatatttgtccagctacaaatcagtgtacaatcgataaaaaccggcgcaagagctgccaggcctgccgacttcggaagtgttacgaagtgggaatggtgaagtgtggctcccggagagagagatgtgggtaccgccttgtgcggagacagagaagtgccgacgagcagctgcactgtgccggcaaggccaagagaagtggcggccacgcgccccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagtaacaagggcatggaacatctgctcaacatgaagtgcaaaaatgtggtcccagtgtatgacctgctgctggagatgctgaatgcccacgtgcttcgcgggtgcaagtcctccatcacggggtccgagtgcagcccggcagaggacagtaaaagcaaagagggctcccagaacccacagtctcagtgacgcctggccctgaggtgaactggcccacagaggtcacaggctgaagcgtgaactccagtgtgtcaggagcctgggcttcatctttctgctgtgtggtccctcatttgg

SEQ ID NO: 89 - protein, 530 aa, Homo sapiens
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

SEQ ID NO: 90 - protein, 9 aa, synthetic construct
  REGION 1..9: note = A cell membrane permeable peptide
RKKRRQRRR

SEQ ID NO: 91 - protein, 21 aa, synthetic construct
  REGION 1..21: note = A cell membrane permeable peptide
TRSSRAGLQFPVGRVHRLLRK

SEQ ID NO: 92 - protein, 27 aa, synthetic construct
  REGION 1..27: note = A cell membrane permeable peptide
GWTLNSAGYLLGKINLKALAALAKKIL

SEQ ID NO: 93 - protein, 18 aa, synthetic construct
  REGION 1..18: note = A cell membrane permeable peptide
KLALKLALKALKAALKLA

SEQ ID NO: 94 - protein, 16 aa, synthetic construct
  REGION 1..16: note = A cell membrane permeable peptide
AAVALLPAVLLALLAP

SEQ ID NO: 95 - protein, 5 aa, synthetic construct
  REGION 1..5: note = A cell membrane permeable peptide
VPMLK

SEQ ID NO: 96 - protein, 5 aa, synthetic construct
  REGION 1..5: note = A cell membrane permeable peptide
PMLKE

SEQ ID NO: 97 - protein, 28 aa, synthetic construct
  REGION 1..28: note = A cell membrane permeable peptide
MANLGYWLLALFVTMWTDVGLCKKRPKP

SEQ ID NO: 98 - protein, 18 aa, synthetic construct
  REGION 1..18: note = A cell membrane permeable peptide
LLIILRRRIRKQAHAHSK

SEQ ID NO: 99 - protein, 21 aa, synthetic construct
  REGION 1..21: note = A cell membrane permeable peptide
KETWWETWWTEWSQPKKKRKV

SEQ ID NO: 100 - protein, 18 aa, synthetic construct
  REGION 1..18: note = A cell membrane permeable peptide
RGGRLSYSRRRFSTSTGR

SEQ ID NO: 101 - protein, 15 aa, synthetic construct
  REGION 1..15: note = A cell membrane permeable peptide
SDLWEMMMVSLACQY

SEQ ID NO: 102 - protein, 12 aa, synthetic construct
  REGION 1..12: note = A cell membrane permeable peptide
TSPLNIHNGQKL

SEQ ID NO: 103 - protein, 16 aa, synthetic construct
  REGION 1..16: note = A cell membrane permeable peptide
RQIKIWFQNRRMKWKK

SEQ ID NO: 104 - protein, 32 aa, synthetic construct
  REGION 1..32: note = PHB2 derived peptide 1-32aa
MAQNLKDLAGRLPAGPRGMGTALKLLLGAGAV

SEQ ID NO: 105 - protein, 37 aa, synthetic construct
  REGION 1..37: note = PHB2 derived peptide 63-99aa
ILAEGLHFRIPWFQYPIIYDIRARPRKISSPTGSKDL

SEQ ID NO: 106 - protein, 21 aa, synthetic construct
  REGION 1..21: note = cross-linked peptide containing PHB2 derived peptide 11-21aa
  REGION 1..13: note = Linked by using hexafluorobenzene
  SITE 10: note = Nle
CRLPAGPRGXGTCRRRRRRRR

SEQ ID NO: 107 - protein, 21 aa, synthetic construct
  REGION 1..21: note = cross-linked peptide containing PHB2 derived peptide 11-21aa
  REGION 1..13: note = Linked by using decafluorobiphenyl
  SITE 10: note = Nle
CRLPAGPRGXGTCRRRRRRRR

SEQ ID NO: 108 - protein, 21 aa, synthetic construct
  REGION 1..21: note = cross-linked peptide containing PHB2 derived peptide 11-21aa
  DISULFID 1..13: note = Intrachain disulfide bond
  SITE 10: note = Nle
CRLPAGPRGXGTCRRRRRRRR

SEQ ID NO: 109 - protein, 21 aa, synthetic construct
  REGION 1..21: note = uncross-linked peptide containing PHB2 derived peptide 11-21aa
  SITE 10: note = Nle
ARLPAGPRGXGTARRRRRRRR

SEQ ID NO: 110 - protein, 23 aa, synthetic construct
  REGION 1..23: note = cross-linked peptide containing PHB2 derived peptide 76-88aa
  REGION 1..15: note = Linked by using hexafluorobenzene
CQYPIIYDIRARPRCRRRRRRRR

SEQ ID NO: 111 - protein, 23 aa, synthetic construct
  REGION 1..23: note = cross-linked peptide containing PHB2 derived peptide 76-88aa
  REGION 1..15: note = Linked by using decafluorobiphenyl
CQYPIIYDIRARPRCRRRRRRRR

SEQ ID NO: 112 - protein, 23 aa, synthetic construct
  REGION 1..23: note = cross-linked peptide containing PHB2 derived peptide 76-88aa
  DISULFID 1..15: note = Intrachain disulfide bond
CQYPIIYDIRARPRCRRRRRRRR

SEQ ID NO: 113 - protein, 23 aa, synthetic construct
  REGION 1..23: note = uncross-linked peptide containing PHB2 derived peptide 76-88aa
AQYPIIYDIRARPRARRRRRRRR

SEQ ID NO: 114 - protein, 21 aa, synthetic construct
  REGION 1..21: note = uncross-linked peptide containing PHB2 derived peptide 11-21aa
ARLPAGPRGMGTARRRRRRRR

SEQ ID NO: 115 - protein, 13 aa, synthetic construct
  REGION 1..13: note = cross-linked peptide containing PHB2 derived peptide 11-21aa
  REGION 1..13: note = Linked by using hexafluorobenzene
  SITE 10: note = Nle
CRLPAGPRGXGTC

SEQ ID NO: 116 - protein, 13 aa, synthetic construct
  REGION 1..13: note = cross-linked peptide containing PHB2 derived peptide 11-21aa
  REGION 1..13: note = Linked by using decafluorobiphenyl
  SITE 10: note = Nle
CRLPAGPRGXGTC

SEQ ID NO: 117 - protein, 13 aa, synthetic construct
  REGION 1..13: note = cross-linked peptide containing PHB2 derived peptide 11-21aa
  DISULFID 1..13: note = Intrachain disulfide bond
  SITE 10: note = Nle
CRLPAGPRGXGTC

SEQ ID NO: 118 - protein, 13 aa, synthetic construct
  REGION 1..13: note = uncross-linked peptide containing PHB2 derived peptide 11-21aa
  SITE 10: note = Nle
ARLPAGPRGXGTA

SEQ ID NO: 119 - protein, 15 aa, synthetic construct
  REGION 1..15: note = cross-linked peptide containing PHB2 derived peptide 76-88aa
  REGION 1..15: note = Linked by using hexafluorobenzene
CQYPIIYDIRARPRC

SEQ ID NO: 120 - protein, 15 aa, synthetic construct
  REGION 1..15: note = cross-linked peptide containing PHB2 derived peptide 76-88aa
  REGION 1..15: note = Linked by using decafluorobiphenyl
CQYPIIYDIRARPRC

SEQ ID NO: 121 - protein, 15 aa, synthetic construct
  REGION 1..15: note = cross-linked peptide containing PHB2 derived peptide 76-88aa
  DISULFID 1..15: note = Intrachain disulfide bond
CQYPIIYDIRARPRC

SEQ ID NO: 122 - protein, 15 aa, synthetic construct
  REGION 1..15: note = uncross-linked peptide containing PHB2 derived peptide 76-88aa
AQYPIIYDIRARPRA

SEQ ID NO: 123 - protein, 19 aa, synthetic construct
  REGION 1..19: note = cross-linked cyclic peptide containing PHB2 derived peptide 11-21aa
  REGION 1..13: note = Linked by using hexafluorobenzene
  SITE 10: note = Nle
  MOD_RES 15: note = 2-Naphthylalanine
CRLPAGPRGXGTCFXRRRR

SEQ ID NO: 124 - protein, 19 aa, synthetic construct
  REGION 1..19: note = cross-linked cyclic peptide containing PHB2 derived peptide 11-21aa
  REGION 1..13: note = Linked by using decafluorobiphenyl
  SITE 10: note = Nle
  MOD_RES 15: note = 2-Naphthylalanine
CRLPAGPRGXGTCFXRRRR

SEQ ID NO: 125 - protein, 19 aa, synthetic construct
  REGION 1..19: note = uncross-linked cyclic peptide containing PHB2 derived peptide 11-21aa
  SITE 10: note = Nle
  MOD_RES 15: note = 2-Naphthylalanine
ARLPAGPRGXGTAFXRRRR

SEQ ID NO: 126 - protein, 21 aa, synthetic construct
  REGION 1..21: note = cross-linked cyclic peptide containing PHB2 derived peptide 76-88aa
  REGION 1..15: note = Linked by using hexafluorobenzene
  MOD_RES 17: note = 2-Naphthylalanine
CQYPIIYDIRARPRCFXRRRR

SEQ ID NO: 127 - protein, 21 aa, synthetic construct
  REGION 1..21: note = cross-linked cyclic peptide containing PHB2 derived peptide 76-88aa
  REGION 1..15: note = Linked by using decafluorobiphenyl
  MOD_RES 17: note = 2-Naphthylalanine
CQYPIIYDIRARPRCFXRRRR

SEQ ID NO: 128 - protein, 21 aa, synthetic construct
  REGION 1..21: note = uncross-linked cyclic peptide containing PHB2 derived peptide 76-88aa
  MOD_RES 17: note = 2-Naphthylalanine
AQYPIIYDIRARPRAFXRRRR

SEQ ID NO: 129 - protein, 20 aa, synthetic construct
  REGION 1..20: note = modified PHB2 peptide
  SITE 8: note = D-alanine
RLPAGPRAMGTARRRRRRRR

SEQ ID NO: 130 - protein, 20 aa, synthetic construct
  REGION 1..20: note = modified PHB2 peptide
  SITE 5: note = D-alanine
RLPAAPRGMGTARRRRRRRR

SEQ ID NO: 131 - protein, 20 aa, synthetic construct
  REGION 1..20: note = modified PHB2 peptide
  SITE 8: note = D-leucine
RLPAGPRLMGTARRRRRRRR

SEQ ID NO: 132 - protein, 20 aa, synthetic construct
  REGION 1..20: note = modified PHB2 peptide
  SITE 5: note = D-leucine
RLPALPRGMGTARRRRRRRR

SEQ ID NO: 133 - protein, 20 aa, synthetic construct
  REGION 1..20: note = modified PHB2 peptide
  SITE 5: note = D-alanine
  SITE 8: note = D-alanine
RLPAAPRAMGTARRRRRRRR

SEQ ID NO: 134 - protein, 20 aa, synthetic construct
  REGION 1..20: note = modified PHB2 peptide
  SITE 5: note = D-leucine
  SITE 8: note = D-leucine
RLPALPRLMGTARRRRRRRR

SEQ ID NO: 135 - protein, 20 aa, synthetic construct
  REGION 1..20: note = modified PHB2 peptide
  SITE 5: note = D-alanine
  SITE 8: note = D-alanine
RLPAAPRLMGTARRRRRRRR

SEQ ID NO: 136 - protein, 20 aa, synthetic construct
  REGION 1..20: note = modified PHB2 peptide
  SITE 5: note = D-leucine
  SITE 8: note = D-alanine
RLPALPRAMGTARRRRRRRR

(19) United States
(12) Patent Application Publication
(10) Pub. No.: US 2026/0209292 A1
(43) Pub. Date: Jul. 23, 2026

(54) THERAPEUTIC AGENT FOR BREAST CANCER COMPRISING BIG3-PHB2 INTERACTION-INHIBITING PEPTIDE DERIVED FROM PHB2

(71) Applicants: TOKUSHIMA UNIVERSITY, Tokushima-shi (JP); ONCOTHERAPY SCIENCE, INC., Kawasaki-shi (JP)

(72) Inventors: TOYOMASA KATAGIRI, Tokushima-shi (JP); TETSURO YOSHIMARU, Tokushima-shi (JP); AKIRA OTAKA, Tokushima-shi (JP); TAKASHI MIYAMOTO, Kawasaki-shi (JP); YASUHIDE OKAMOTO, Kawasaki-shi (JP)

(73) Assignees: TOKUSHIMA UNIVERSITY, Tokushima-shi (JP); ONCOTHERAPY SCIENCE, INC., Kawasaki-shi (JP)

(21) Appl. No.: 19/546,921

(22) Filed: Feb. 23, 2026

Related U.S. Application Data
(63) Continuation of application No. 17/297,986, filed on Nov. 28, 2019, now Pat. No. 12,595,289.

(30) Foreign Application Priority Data
Nov. 30, 2018 (JP) 2018-225660

(51) Int. Cl. C07K 14/47; A61K 38/00; A61P 35/00

(52) U.S. Cl. CPC C07K 14/4703; A61P 35/00; C07K 14/4748; A61K 38/00

(57) ABSTRACT

The present invention provides peptides containing the BIG3 polypeptide-binding site in a PHB2 polypeptide, which inhibit the binding between a PHB2 polypeptide and a BIG3 polypeptide, and pharmaceutical compositions containing the peptide. The peptides of the present invention have the ability to bind not to PHB2, whose expression is found in organs throughout the human body, but to BIG3, which is a protein highly expressed specifically in particularly estrogen receptor-positive cancer, and have excellent growth suppressive effects on BIG3-positive cancer cells. Accordingly, the peptides of the present invention are useful as therapeutic agents for breast cancer which can avoid expression of side effects.

### CROSS-REFERENCE TO RELATED APPLICATIONS

The present application is a continuation of U.S. patent application Ser. No. 17/297,986, filed May 27, 2021, which is a § 371 National Phase Application of PCT/JP2019/046505, filed Nov. 28, 2019, which application claims the benefit of Japanese Patent Application No. JP 2018-225660, filed Nov. 30, 2018, the entire contents of which are incorporated by reference in their entireties for all purposes herein.

### SEQUENCE LISTING

A Sequence Listing conforming to the rules of WIPO Standard ST.26 is submitted electronically herewith via Patent Center and is hereby incorporated by reference in its entirety. The Sequence Listing file, identified as 087331-1253518-031000US.xml, is 225,558 bytes in size, and was created on Mar. 5, 2026.

### TECHNICAL FIELD

The present invention relates to PHB2-derived peptides that inhibit BIG3-PHB2 interaction and therapeutic agents for breast cancer comprising the peptide.

### BACKGROUND ART

Estrogen-receptor α (ERα) plays a key role in the development and progression of breast cancer. The current endocrine therapies for breast cancer mainly target ERα signaling, and use selective ERα modulators (for example, tamoxifen and raloxifene), ERα down-regulators (for example, fulvestrant), and aromatase inhibitors (AI) (NPLs 1 to 3). Among these therapies, a method that uses tamoxifen, which inhibits breast cancer cell proliferation through competitive binding to ERα, is a standard therapy for patients with ERα-positive breast cancer. However, tamoxifen therapy is often ineffective, and the patient may die from recurrent endocrine therapy-resistant tumors (NPLs 4 and 5). Furthermore, compared with tamoxifen, AI, which blocks estrogen synthesis, provides substantial clinical effects such as good efficacy, significant increase in relapse-free survival period, and a prolonged time to disease recurrence in postmenopausal women; however, some patients who have undergone AI treatment still relapse (NPLs 6 and 7). The precise molecular events having effects on the efficacy of these endocrine therapies remain unknown.

A complex formed between brefeldin A-inhibited guanine nucleotide-exchange protein 3 (BIG3), which is a cancer specific protein, and prohibitin 2 (PHB2), which is a tumor suppressor, plays a key role in estrogen signaling regulation in ERα-positive breast cancer (NPLs 8 and 9). BIG3 binds to PHB2 to inhibit the ability of PHB2, which suppresses the estrogen-dependent transcriptional activation, and thereby causes constitutive ERα activation.

Based on these findings, strategies of making PHB2 exhibit its tumor suppressive activity by dissociating PHB2 from its complex with BIG3 through inhibition of the BIG3-PHB2 interaction, may become a novel therapy for breast cancer. Based on this strategy, the present inventors have previously developed a dominant negative peptide of BIG3, which specifically inhibits the BIG3-PHB2 interaction (PTL 1). This peptide has been confirmed to suppress breast cancer growth by reactivating the tumor suppressive activity of PHB2 to inhibit ERα-signaling pathways that bring about the growth of breast cancer (PTL 1).

### CITATION LIST

### Patent Literature

[PTL 1] WO 2013/018690

### Non-Patent Literature

[NPL 1] Johnston, S. R., Clin. Cancer Res. 16(7), 1979-87 (2010).
[NPL 2] Fisher, B. et al., J. Natl. Cancer Inst. 97(22), 1652-62 (2005).
[NPL 3] Jordan, V. C., Nature Rev. Drug Discov. 2(3), 205-13 (2003).
[NPL 4] Clarke, R. et al., Pharmacol. Rev. 53(1), 25-71 (2001).
[NPL 5] Fisher, B. et al., J. Natl. Cancer Inst. 93(9), 684-90 (2001).
[NPL 6] Chlebowski, R. et al., Breast 2, S1-11 (2009).
[NPL 7] Chumsri, S. et al., J. Steroid Biochem. Mol. Biol. 125(1-2), 13-22 (2011).
[NPL 8] Kim, J. W. et al., Cancer Sci. 100(8), 1468-78 (2009).
[NPL 9] Yoshimaru, T. et al., Nat. Commun. 4, 2443 (2013).
[NPL 10] Yoshimaru, T. et al., Sci Rep. 7(1), 1821 (2017).

### SUMMARY OF INVENTION

### Technical Problem

As noted above, it has been elucidated that an estrogen receptor (ER) activation regulator, BIG3, interacts with a suppressor, PHB2, to cause constitutive activation of ER and that a BIG3-PHB2 interaction-targeting inhibitory peptide (ERAP; the amino acid sequence at positions 165 to 177 (QMLSDLTLQLRQR; SEQ ID NO: 33) of the BIG3 protein) has the effect of suppressing estrogen (E2)-dependent breast cancer cell growth (PTL 1; WO2017/126461). However, although ERAP, derived from the BIG3 sequence, achieves the interaction inhibition by binding to PHB2, it cannot be denied that ERAP exerts non-selective effects in organs other than cancer tissue because PHB2 expression is found in organs throughout the human body.

Accordingly, an objective of the present invention is to provide a therapeutic strategy which targets the BIG3-PHB2 interaction and can be expected to be highly selective for breast cancer.

### Solution to Problem

The present inventors designed multiple PHB2-derived peptides (PHB2 peptides) based on the data of candidate protein interaction regions on the PHB2 amino acid sequence predicted through in silico analysis, and used these PHB2 peptides for screening to identify interaction regions using the effect of suppressing cell growth as an indicator. As a result, the present inventors succeeded in finding that PHB2 peptide No. 1 (11-RLPAGPRGMGTA-22 (SEQ ID NO: 1)) and PHB2 peptide No. 5 (76-QYPIIYDIRARPRKI-90 (SEQ ID NO: 5)) each have the effect of suppressing growth by about 50%, and in particular that the combination of PHB2 peptides Nos. 1 and 5 exhibits the effects of suppressing growth by more than 90% and inhibiting the BIG3-PHB2 interaction, as with ERAP. Furthermore, these effects were also observed for peptides consisting of sequences around PHB2 peptides Nos. 1 and 5 and for peptides in which amino acid residues at various positions in PHB2 peptides Nos. 1 and 5 have been substituted.

On the other hand, the cell growth suppressive effect of PHB2 peptides Nos. 1 and 5 was also observed on triple-negative breast cancer cells which do not express estrogen receptors and such but express BIG3. Moreover, the use of these peptides in combination showed enhancement of the effect. It is considered that in triple-negative breast cancer, which proliferates in a manner independent of proliferative signals such as hormones, its growth is activated by the binding between PHB2 and BIG3 without receipt of these signals in cells. It was suggested that the PHB2-derived peptides may suppress cell growth by inhibiting the binding between BIG3 and PHB2 in breast cancer expressing at least BIG3.

The present inventors thus found the PHB2-derived peptides which inhibit the BIG3-PHB2 interaction and exert an antitumor effect on E2-dependent breast cancer and triple-negative breast cancer, and completed the present invention. That is, the present invention provides the following peptides and uses thereof.
[1] A peptide, comprising a site binding to a BIG3 polypeptide in a PHB2 polypeptide, wherein the peptide inhibits the binding between the PHB2 polypeptide and the BIG3 polypeptide.
[2] The peptide of [1], wherein the peptide comprises any one or a combination of all or part of the amino acid sequence consisting of the amino acids at positions 11 to 21; all or part of the amino acid sequence consisting of the amino acids at positions 76 to 88; and all or part of the amino acid sequence consisting of the amino acids at positions 44 to 57, in the amino acid sequence of SEQ ID NO: 28 (full-length PHB2 polypeptide).
[3] A peptide, comprising an amino acid sequence selected from the group consisting of (a) to (f) below, wherein the peptide inhibits the binding between a PHB2 polypeptide and a BIG3 polypeptide:
(a) an amino acid sequence selected from the group consisting of SEQ ID NOs: 1 and 36 to 41 (PHB2 sequence-derived peptides Nos. 1 and 36 to 41);
(b) an amino acid sequence in which one, two, or several amino acids are substituted, deleted, inserted and/or added in the amino acid sequence selected from the group consisting of SEQ ID NOs: 1 and 36 to 41 (PHB2 sequence-derived peptides Nos. 1 and 36 to 41);
(c) an amino acid sequence selected from the group consisting of SEQ ID NOs: 5 and 47 to 53 (PHB2 sequence-derived peptides Nos. 5 and 47 to 53);
(d) an amino acid sequence in which one, two, or several amino acids are substituted, deleted, inserted and/or added in the amino acid sequence selected from the group consisting of SEQ ID NOs: 5 and 47 to 53 (PHB2 sequence-derived peptides Nos. 5 and 47 to 53);
(e) an amino acid sequence selected from the group consisting of SEQ ID NOs: 82 and 83 (PHB2 sequence-derived peptides Nos. 82 and 83); and
(f) an amino acid sequence in which one, two, or several amino acids are substituted, deleted, inserted and/or added in the amino acid sequence selected from the group consisting of SEQ ID NOs: 82 and 83 (PHB2 sequence-derived peptides Nos. 82 and 83).
[4] The peptide of [3], wherein the peptide comprises an amino acid sequence selected from the group consisting of (a′) and (b′) below:
(a′) an amino acid sequence in which one, two, or several amino acid residues located at positions other than those corresponding to glycine (Gly/G) at positions 15 and 18 in the amino acid sequence of SEQ ID NO: 28 (full-length PHB2 polypeptide) are substituted with other amino acid residues in an amino acid sequence selected from the group consisting of SEQ ID 15 NOs: 1 and 36 to 41 (PHB2 sequence-derived peptides Nos. 1 and 36 to 41); and
(b′) an amino acid sequence in which one, two, or several amino acid residues located at positions other than that corresponding to aspartic acid (Asp/D) at position 82 in the amino acid sequence of SEQ ID NO: 28 (full-length PHB2 polypeptide) are substituted with other amino acid residues in an amino acid sequence selected from the group consisting of SEQ ID NOs: 5 and 47 to 53 (PHB2 sequence-derived peptides Nos. 5 and 47 to 53).
[5] The peptide of any one of [1] to [4], wherein the peptide consists of 80 amino acid residues or less.
[6] The peptide of any one of [1] to [5], wherein the peptide consists of 25 amino acid residues or less.
[7] The peptide of any one of [1] to [6], wherein the peptide consists of an amino acid sequence selected from the group consisting of SEQ ID NOs: 1, 5, 36 to 41, 47 to 53, 82, and 83 (PHB2 sequence-derived peptides Nos. 1, 5, 36 to 41, 47 to 53, 82, and 83).
[8] The peptide of any one of [1] to [7], wherein the peptide has been modified with a cell membrane-permeable substance.
[9] The peptide of any one of [1] to [8], wherein the peptide is cyclic.
[10] The peptide of any one of [1] to [9], wherein the peptide is cross-linked.
[11] The peptide of any one of [1] to [10], wherein the peptide has either or both of the following properties (i) and (ii):
(i) suppressing growth of BIG3-positive cells; and
(ii) promoting phosphorylation of a serine residue in the PHB2 polypeptide in BIG3-positive cells.
[12] A polynucleotide encoding the peptide of any one of [1] to [11].
[13] A pharmaceutical composition comprising: at least one ingredient selected from the group consisting of one or more of the peptides of any one of [1] to [11], a polynucleotide(s) encoding the peptide(s), and a pharmaceutically acceptable salt(s) of the peptide(s); and a pharmaceutically acceptable carrier.
[14] The pharmaceutical composition of [13], wherein the composition comprises any one or a combination of: a peptide comprising all or part of the amino acid sequence consisting of the amino acids at positions 11 to 21 in the amino acid sequence of SEQ ID NO: 28 (full-length PHB2 polypeptide); a peptide comprising all or part of the amino acid sequence consisting of the amino acids at positions 44 to 57 in the amino acid sequence of SEQ ID NO: 28 (full-length PHB2 polypeptide); and a peptide comprising all or part of the amino acid sequence consisting of the amino acids at positions 76 to 88 in the amino acid sequence of SEQ ID NO: 28 (full-length PHB2 polypeptide).
[15] The pharmaceutical composition of or [14], which is for suppressing growth of cancer cells or for treatment and/or prophylaxis (prevention) of cancer.
[16] The pharmaceutical composition of [15], wherein the cancer is BIG3-positive cancer.
[17] The pharmaceutical composition of or [16], wherein the cancer is breast cancer.
[18] The pharmaceutical composition of any one of to [17], wherein the cancer is estrogen receptor-positive cancer.
[19] A method for either or both of treatment and prophylaxis (prevention) of cancer, wherein the method comprises administering to a subject at least one selected from the group consisting of one or more of the peptides of any one of [1] to [11]; a polynucleotide(s) encoding the peptide(s); and a pharmaceutically acceptable salt(s) of the peptide(s).
[20] The method of [19], wherein the method comprises administering any one or a combination of: a peptide comprising all or part of the amino acid sequence consisting of the amino acids at positions 11 to 21 in the amino acid sequence of SEQ ID NO: 28 (full-length PHB2 polypeptide); a peptide comprising all or part of the amino acid sequence consisting of the amino acids at positions 44 to 57 in the amino acid sequence of SEQ ID NO: 28 (full-length PHB2 polypeptide); and a peptide comprising all or part of the amino acid sequence consisting of the amino acids at positions 76 to 88 in the amino acid sequence of SEQ ID NO: 28 (full-length PHB2 polypeptide).
[21] A method for either or both of treatment and prophylaxis (prevention) of pharmacotherapy-resistant breast cancer (for example, triple-negative breast cancer), wherein the method comprises: selecting a patient with pharmacotherapy-resistant breast cancer (for example, a patient with triple-negative breast cancer); and administering to a subject at least one selected from the group consisting of one or more of the peptides of any one of [1] to [11], a polynucleotide(s) encoding the peptide(s), and a pharmaceutically acceptable salt(s) of the peptide(s).

Alternatively, the present invention provides use of at least one selected from the group consisting of one or more of the peptides of any one of [1] to [11] mentioned above, a polynucleotide(s) encoding the peptide(s), and a pharmaceutically acceptable salt(s) of the peptide(s), in the manufacture of a pharmaceutical composition for either or both of treatment and prophylaxis (prevention) of cancer. Furthermore, the present invention relates to at least one selected from the group consisting of one or more of the peptides of any one of [1] to mentioned above, a polynucleotide(s) encoding the peptide(s), and a pharmaceutically acceptable salt(s) of the peptide(s), for use in either or both of treatment and prophylaxis (prevention) of cancer. Moreover, the present invention relates to methods of manufacturing a pharmaceutical composition for either or both of treatment and prophylaxis (prevention) of cancer, the method comprising mixing or compounding with a carrier at least one selected from the group consisting of one or more of the peptides of any one of [1] to [11] mentioned above, a polynucleotide(s) encoding the peptide(s), and a pharmaceutically acceptable salt(s) of the peptide(s).

### Advantageous Effects of Invention

The peptides of the present invention have the ability to bind to BIG3, a protein highly expressed specifically in, among others, estrogen receptor-positive cancer, and not to PHB2, of which expression is observed in organs throughout the human body, and can inhibit the BIG3-PHB2 interaction. Thus, the peptides of the present invention can be expected to have high selectivity for estrogen receptor-positive cancer.

Furthermore, the peptides of the present invention exhibit a growth suppressive effect not only on estrogen-dependent breast cancer cells but also on triple-negative breast cancer cells. For triple-negative breast cancer, there has so far been no effective molecular target drug, and treatment with existing anticancer agents having strong side effects has been the only option. On the other hand, the cell growth suppressive effect of the peptides of the present invention was not observed in normal mammary gland epithelial cells which did not express BIG3. These suggest that the peptides of the present invention are useful as therapeutic drugs for BIG3-positive cancer, regardless of whether the cancer is hormone-dependent or not.

### BRIEF DESCRIPTION OF DRAWINGS

FIG. 1A: screening for PHB2 sequence-derived peptides which suppress estrogen-dependent cell growth is shown. Human breast cancer cell line MCF-7 was treated with 10 μM each of PHB2 sequence-derived peptides, and then immediately stimulated with 10 nM estrogen to evaluate the cell number for 24 hours by MTT assay. The data represents mean±standard deviation of three independent experiments. The full-length sequence of human PHB2 protein is shown below the graph, and synthetic peptides in which eight arginine residues were added to the C terminus of the underlined sequences were used in the assay. Moreover, the bold letters represent amino acids which are suggested to be involved in the interaction with BIG3 by in silico analysis, boxed “S” represents the phosphorylation site of PHB2, and the broken line represents a region whose binding to BIG3 has been demonstrated.

FIGS. 1B-1C: MCF-7 was treated with 10 μM each of PHB2 sequence-derived peptides surrounding 11-22aa (FIG. 1B) and 76-90aa (FIG. 1C) (SEQ ID NOs: 1, 34 to 43, 5, and 44 to 55), and then immediately stimulated with 10 nM estrogen to evaluate the cell number for 24 hours by MTT assay. The data represents mean±standard deviation of three independent experiments. The positions of the PHB2-derived sequences contained in the synthetic peptides used in the assay are shown on the left side of the graph.

FIG. 1D: MCF-7 was treated with the combinations of 10 μM PHB2 sequence-derived peptide 11-22aa with 10 μM each of PHB2 sequence-derived peptides surrounding 76-90aa, and then the cells were immediately stimulated with 10 nM estrogen to evaluate the cell number for 24 hours by MTT assay. FIG. 1E: MCF-7 was treated with the combinations of 10 μM PHB2 sequence-derived peptide 76-90aa with 10 μM each of PHB2 sequence-derived peptides surrounding 11-22aa, and then the cells were immediately stimulated with 10 nM estrogen to evaluate the cell number for 24 hours by MTT assay. The data represents mean±standard deviation of three independent experiments.

FIGS. 2A-2C show the effect by combination of PHB2 peptides 11-22aa and 76-90aa on the suppression of estrogen-dependent growth. FIG. 2A: Human breast cancer cell line MCF-7 was treated with 10 μM each of ERAP (positive control), PHB2 peptides 11-22aa, 76-90aa, and 86-100aa (negative control), a combination of 11-22aa and 76-90aa, and a combination of 76-90aa and 86-100aa, and then the cells were immediately stimulated with 10 nM estrogen to evaluate the cell number after 24 hours by MTT assay. The data represents mean±standard deviation of three independent experiments. FIG. 2B: The suppressive effects of PHB2 peptides 11-22aa and 76-90aa and their combination on the interaction between BIG3 and PHB2 in MCF-7 were evaluated by Western blotting. MCF-7 was treated with 20 μM and 50 μM each of PHB2 peptides for 24 hours, and the cells were then lysed and immunoprecipitated with an anti-BIG3 antibody to perform immunoblot analysis using the antibodies shown in the figure. The percent binding inhibition is represented as the ratio when taking the PHB2 band area in untreated cells as 100. FIG. 2C: Human breast cancer cell line MCF-7 was treated with 10 μM each of ERAP, PHB2 peptides 11-22aa, 76-90aa, and 86-100aa, a combination of 11-22aa and 76-90aa, and a combination of 76-90aa and 86-100aa, and then the cells were immediately stimulated with 10 nM estrogen to evaluate the phosphorylation of PHB2 (Ser39) after 24 hours by Western blotting. The strength of phosphorylation is represented as the ratio when taking the phosphorylated band area of ERAP treatment in the presence of estrogen as 1.0.

FIGS. 3A-3C show the effect of PHB2 peptide 11-90aa on estrogen-dependent growth and on the interaction between BIG3 and PHB2. FIG. 3A: Human breast cancer cell line MCF-7 was treated with PHB2 peptides 11-90aa (10, 20, and 50 μM), 11-22aa (50 M), and 76-90aa (50 uM), and a combination of 11-22aa and 76-90aa, and then the cells were immediately stimulated with 10 nM estrogen to evaluate the cell number after 24 hours by MTT assay. The data represents mean±standard deviation of three independent experiments. FIG. 3B: The inhibition effects of PHB2 peptides 11-90aa and 10 μM ERAP (positive control) on the interaction between BIG3 and PHB2 in MCF-7 were evaluated by Western blotting. FIG. 3C: Human breast cancer cell line MCF-7 was treated with PHB2 peptide 11-90aa (20, 50, and 100 μM) and 10 μM ERAP, and then the cells were immediately stimulated with 10 nM estrogen to evaluate the phosphorylation of PHB2 (Ser39) after 24 hours by Western blotting. The strength of phosphorylation is represented as the ratio when taking the phosphorylated band area of the untreated cells as 1.0.

FIGS. 4A-4C show schemes of branched and cyclic PHB2 peptides. FIG. 4A: Linear bound PHB2 peptide. FIG. 4B: Branched PHB2 peptide. FIG. 4C: Cyclic PHB2 peptides.

FIGS. 5A-5C show the suppressive effects of branched and cyclic PHB2 peptides on estrogen-dependent growth. FIG. 5A: Human breast cancer cell line MCF-7 was treated with 10 μM each of linear peptides 11-22aa and 76-90aa, a combination of linear 11-22aa and 76-90aa, the linear bound peptide, the branched bound peptide, cyclic 11-21aa and cyclic 76-88aa, and a combination of the cyclic peptides, and then the cells were immediately stimulated with 10 nM estrogen to evaluate the cell number after 24 hours by MTT assay. The data represents mean #standard deviation of three independent experiments. FIG. 5B shows the results of MTT assay showing that linear 11-22aa and linear 76-90aa, the linear bound peptide, the branched bound peptide, and cyclic 11-21aa and cyclic 76-88aa peptides have no effect on growth of normal mammary gland epithelial cells, MCF-10A. FIG. 5C: Human breast cancer cell line MCF-7 was treated with 10 UM each of linear peptides 11-22aa and 76-90aa, a combination of linear 11-22aa and 76-90aa, the linear bound peptide, the branched bound peptide, cyclic 11-21aa and cyclic 76-88aa, and a combination of the cyclic peptides, and their suppressive effects on the interaction between BIG3 and PHB2 were evaluated by Western blotting.

FIGS. 6A-6B show the effects of suppressing estrogen-dependent growth by a combination of cyclic PHB2 peptides. FIG. 6A: Human breast cancer cell line MCF-7 was treated with linear 11-22aa and cyclic 11-21aa (left) or with linear 76-90aa and cyclic 76-88aa (right), and then the cells were immediately stimulated with 10 nM estrogen to evaluate the suppressive effect by MTT assay every 24 hours up to 96 hours. FIG. 6B represents the results of MTT assay showing that cyclic 11-21aa and cyclic 76-88aa peptides have no effect on the growth of normal mammary gland epithelial cells, MCF-10A. The data represents mean±standard deviation of three independent experiments.

FIGS. 7A-7C show the concentration-dependent suppressive effects of cyclic PHB2 peptides on estrogen-dependent growth. FIG. 7A: Human breast cancer cell line MCF-7 was treated with 0.1, 0.5, 1, 2.5, 5, 10, and 20 μM each of cyclic 11-21aa and 76-88aa, and then the cells were immediately stimulated with 10 nM estrogen to evaluate the cell number after 24 hours by MTT assay. The data represents mean±standard deviation of three independent experiments. FIG. 7B: Human breast cancer cell line MCF-7 was treated with cyclic 11-21aa (4 μM), cyclic 76-88aa (2 μM), and their combination, and then the cells were immediately stimulated with 10 nM estrogen to evaluate the suppressive effect by MTT assay every 24 hours up to 96 hours. The data represents mean±standard deviation of three independent experiments. FIG. 7C represents the results of MTT assay showing that cyclic 11-21aa (4 μM), cyclic 76-88aa (2 μM), and their combination have no effect on growth of normal mammary gland epithelial cells, MCF-10A.

FIGS. 8A-8C: PHB2 amino acids important for suppressing estrogen-dependent growth are shown. FIG. 8A depicts alanine-mutated PHB2 peptides of No. 1 (11-22aa) and No. 5 (76-90aa). FIGS. 8B, 8C: Human breast cancer cell line MCF-7 was treated with 10 UM each of the alanine-mutated peptides derived from PHB2 sequence, and then the cells were immediately stimulated with 10 nM estrogen to evaluate the cell number for 24 hours by MTT assay. The data represents mean±standard deviation of three independent experiments.

FIG. 8D depicts PHB2 peptides in which 51-57 aa has been added to PHB2 peptides No. 2 (42-50aa) and No. 3 (38-50aa). FIG. 8E: The PHB2 sequence-derived peptides of FIG. 8D were each added at 10 μM, and the cells were immediately stimulated with 10 nM estrogen to evaluate the cell number for 24 hours by MTT assay. The data represents mean±standard deviation of three independent experiments.

FIGS. 9A-9C show the cell growth suppressive effects of PHB2 peptides 11-22aa (FIG. 9A) and 76-90aa (FIG. 9B) on triple-negative breast cancer cells. Each peptide was diluted in a 3-fold dilution series starting from 20 μM with total 11 concentrations and added to breast cancer cell line MDA-MB-231. The numbers of viable cells were measured 96 hours after the peptide addition, the relative values were calculated based on negative control cells to which no peptide was added, and the values were plotted on the graph. The data represents mean±standard deviation of three independent experiments. FIG. 9C shows the results of examining the combined effect of PHB2 peptides 11-22aa and 76-90aa on cell growth of MDA-MB-231. The peptides were added to the cells alone or in combination at IC50 value, the numbers of viable cells were measured after 96 hours, the relative values were calculated based on negative control cells to which phosphate buffered saline (PBS) was added, and the values were plotted on the graph. The data represents mean±standard deviation of three independent experiments.

FIGS. 10A-10B: the suppressive effects of cross-linked PHB2 peptides on estrogen-dependent growth are shown. FIG. 10A shows schemes of cross-linking forms. FIG. 10B shows PHB2 peptides prepared by adding cysteine to both ends of PHB2 peptides 11-21aa and 76-88aa and cross-linking them (SEQ ID NOs: 106 to 108, 110 to 112, 115 to 117, and 119 to 121). The PHB2 peptides of SEQ ID NOs: 109, 113, 114, 118, and 122 were prepared as non-cross-linked peptides by adding alanine to both ends. The PHB2 peptides of SEQ ID NOs: 106 to 114 were prepared by adding polyarginine to the C terminus. Furthermore, in the PHB2 peptides of 11-21aa, methionine at position 19 in the amino acid sequence of SEQ ID NO: 28 (full-length PHB2 polypeptide) was substituted with norleucine (Nle) to avoid oxidation during synthesis.

FIG. 10C: Human breast cancer cell line MCF-7 was treated with cross-linked PHB2 peptides of 11-21aa (left panel) or with cross-linked PHB2 peptides of 76-88aa (right panel), and then the cells were immediately stimulated with 10 nM estrogen to evaluate the cell number after 24 hours by MTT assay. The data represents mean±standard deviation of three independent experiments.

FIGS. 11A-11B show the suppressive effects of cyclic PHB2 peptides on estrogen-dependent growth. FIG. 11A depicts cross-linking types of PHB2 peptides of cyclic 11-21aa (SEQ ID NO: 25) and cyclic 76-88aa (SEQ ID NO: 26). The cyclic PHB2 peptides of SEQ ID NOs: 125 and 128 were prepared as non-cross-linked cyclic peptides by adding alanine to both ends of PHB peptides 11-21aa and 76-88aa. All cyclic PHB2 peptides were prepared by adding an unnatural amino acid and consecutive multiple arginine residues to the C terminus. Furthermore, in the cyclic PHB2 peptides of 11-21aa, methionine at position 19 in the amino acid sequence of SEQ ID NO: 28 (full-length PHB2 polypeptide) was substituted with norleucine (Nle) to avoid oxidation during synthesis. FIG. 11B: Human breast cancer cell line MCF-7 was treated with cyclic PHB2 peptides, and then the cells were immediately stimulated with 10 nM estrogen to evaluate the cell number after 24 hours by MTT assay. The data represents mean±standard deviation of three independent experiments.

FIGS. 12A-12B show the effects of modifications of PHB2 peptide 11-22aa on estrogen-dependent growth. FIG. 12A depicts PHB2 peptides prepared from the PHB2 peptide of SEQ ID NO: 1 (11-22aa) by substituting glycine at positions 15 and 18 in the amino acid sequence of SEQ ID NO: 28 (full-length PHB2 polypeptide) with D-alanine and D-leucine. FIG. 12B: Human breast cancer cell line MCF-7 was treated with the modified PHB2 peptides of 11-22aa, and then the cells were immediately stimulated with 10 nM estrogen to evaluate the cell number after 24 hours by MTT assay. The data represents mean±standard deviation of three independent experiments.

FIG. 13 shows the effects of cross-linked PHB2 peptides on normal mammary gland epithelial cells, MCF-10A. The figure represents the results of MTT assay showing that cross-linked PHB2 peptides and cyclic PHB2 peptides have no effect on growth of normal mammary gland epithelial cells, MCF-10A. The data represents mean±standard deviation of three independent experiments.

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

### Definitions

The words “a”, “an”, and “the” used herein mean “at least one” unless otherwise specifically indicated.

Herein, unless otherwise specifically indicated, amino acids represented by capital letters indicate L-amino acids. Amino acids represented by lower-case letters indicate D-amino acids. Furthermore, L-amino acids and D-amino acids represented herein may include amino acids in which any of amino group, carboxyl group, and side chains has been modified. Examples of preferred modifications include acetylation of the amino group, amidation of the carboxyl group, tag peptide addition such as FLAG-tagging and HA-tagging, and such. Herein, numbers indicating the positions of amino acid residues in amino acid sequences have been given with the N-terminal amino acid residue as number 1 and in order toward the C terminus, unless otherwise specifically indicated.

The term “BIG3” used herein refers to brefeldin A-inhibited guanine nucleotide-exchange protein 3. BIG3 forms a complex with PHB2 to inhibit the estrogen-dependent transcriptional activation-suppressing function of PHB2. BIG3 is also referred to as “ARFGEF family member 3 (ARFGEF3)” or “A7322”. An example of a representative nucleotide sequence of the human BIG3 gene is shown in SEQ ID NO: 31 (GenBank Accession No. NM_020340.4), and the amino acid sequence encoded by the gene is shown in SEQ ID NO: 32. In the present invention, BIG3 is not limited to that encoded by the aforementioned nucleotide sequence and also encompasses their isoforms and mutants.

The term “PHB2” used herein refers to prohibitin 2. PHB2 binds to estrogen receptors to inhibit estrogen receptor signaling pathways and suppresses estrogen-dependent cell growth. PHB2 is also referred to as “Repressor of Estrogen Activity (REA)”. Examples of representative nucleotide sequences of the human PHB2 gene are shown in SEQ ID NO: 27 (GenBank Accession No. NM_001144831.1) and SEQ ID NO: 29 (GenBank Accession No. NM_001267700.1), and the amino acid sequences encoded by the genes are shown in SEQ ID NO: 28 and SEQ ID NO: 30, respectively. In the present invention, PHB2s are not limited to those encoded by the aforementioned nucleotide sequences and also encompass their isoforms and mutants.

The term “estrogen receptor” used herein encompasses both estrogen receptor α (ERα) and estrogen receptor β (ERβ). ERα and ERB are encoded by the ESRI gene and ESR2 gene, respectively. The nucleotide sequence of a representative human ESRI gene and the amino acid sequence of a representative human ERα are shown in SEQ ID NO: 86 (GenBank Accession No. NM_000125.3) and SEQ ID NO: 87 (GenBank Accession No. NP_000116.2), respectively. Furthermore, the nucleotide sequence of a representative human ESR2 gene and the amino acid sequence of a representative human ERB are shown in SEQ ID NO: 88 (GenBank Accession No. NM_001437.2) and SEQ ID NO: 89 (GenBank Accession No. NP_001428.1), respectively. In the present invention, however, the nucleotide sequences and amino acid sequences of estrogen receptor are not limited thereto and also encompass their isoforms and mutants. In a preferred embodiment, the estrogen receptor is ERα. It has been reported that transcriptional activation of ERα and ERβ is both regulated by a PHB2 polypeptide (Montano M M, et al., Proc Natl Acad Sci USA. 96(12): 6947-52 (1999)).

Herein, the term “estrogen receptor-positive” used in the context of a cell or cancer means that a cell or a cancer cell constituting cancer expresses an estrogen receptor. Whether a cell or cancer is estrogen receptor-positive or not can be confirmed by a known method such as ELISA and immunohistochemical staining. Furthermore, herein, the term “estrogen receptor-negative” used in the context of a cell or cancer means that a cell or a cancer cell constituting cancer does not express an estrogen receptor.

The term “ERAP” used herein refers to a peptide consisting of the amino acid sequence of SEQ ID NO: 33. The amino acid sequence of SEQ ID NO: 33 is a sequence consisting of the 165th to 177th amino acid residues in the amino acid sequence of BIG3 (SEQ ID NO: 32), and contains amino acid residues important for binding with PHB2 (glutamine (Q) at position 165, aspartic acid (D) at position 169, and glutamine (Q) at position 173 in the amino acid sequence of SEQ ID NO: 32). ERAP has an ability to bind to PHB2 and inhibits BIG3 from forming the complex with PHB2 by binding competitively to PHB2.

The term “treatment” used herein encompasses alleviation/improvement of at least one symptom caused by a target disease, suppression of progression of the disease, suppression of enlargement of the disease site, and such. For example, “cancer treatment (treatment of cancer)” includes cancer cell growth suppression, suppression of cancer progression, induction of regression/remission of cancer, alleviation/improvement of symptoms accompanying cancer, suppression of cancer metastasis, suppression of postoperative recurrence, and induction of prolonged survival time.

### 1. PHB2 Peptides

The present invention provides peptides comprising a site binding to a BIG3 polypeptide (a BIG3 polypeptide-binding site) in a PHB2 polypeptide, which inhibit the binding between the PHB2 polypeptide and the BIG3 polypeptide. The peptides of the present invention are also herein referred to as “PHB2 peptides”, “PHB2-derived peptides”, or “PHB2 sequence-derived peptides”.

The peptides of the present invention have the ability to bind to a BIG3 polypeptide by comprising the BIG3 polypeptide-binding site in a PHB2 polypeptide. Consequently, the peptides competitively inhibit the binding of the PHB2 polypeptide to the BIG3 polypeptide. The PHB2 peptides of the present invention can be salts as long as they have the effect of inhibiting the binding between a PHB2 polypeptide and a BIG3 polypeptide. For example, the PHB2 peptides can be salts with acids (such as inorganic acids and organic acids) or with bases (such as alkaline metals, alkaline earth metals, and amines). The salts with acids include, for example, salts with inorganic acids (for example, hydrochloric acid, phosphoric acid, hydrobromic acid, sulfuric acid, and acetic acid) and those with organic acids (for example, acetic acid, formic acid, propionic acid, fumaric acid, maleic acid, succinic acid, tartaric acid, citric acid, malic acid, oxalic acid, benzoic acid, methanesulfonic acid, benzenesulfonic acid, and meglumine acid). The salts with bases include, for example, salts with sodium, potassium, calcium, and ammonium. Preferred examples of salts of the peptides of the present invention include acetates, hydrochlorides, meglumine salts, and ammonium salts.

The “site binding to a BIG3 polypeptide in a PHB2 polypeptide (BIG3 polypeptide-binding site in a PHB2 polypeptide)” means an amino acid residue(s) involved in binding to a BIG3 polypeptide in the amino acid sequence constituting a PHB2 polypeptide. Such an amino acid residue(s) includes, for example, glycine at positions 15 and 18 and aspartic acid at position 82 in the amino acid sequence of SEQ ID NO: 28 (full-length PHB2 polypeptide). Thus, in a preferred embodiment, the peptides of the present invention are peptides which comprise glycine at positions 15 and 18 and aspartic acid at position 82 in the amino acid sequence of SEQ ID NO: 28 (full-length PHB2 polypeptide) and inhibit the binding between a PHB2 polypeptide and a BIG3 polypeptide. Herein, the number of a particular amino acid residue in an amino acid sequence indicates the number of the amino acid residue counted from the N terminus.

Examples of amino acid sequences comprising the BIG3 polypeptide-binding site in a PHB2 polypeptide include (a) all or part of the amino acid sequence consisting of the amino acids at positions 11 to 21 (SEQ ID NO: 84), (b) all or part of the amino acid sequence consisting of the amino acids at positions 76 to 88 (SEQ ID NO: 85), and (c) all or part of the amino acid sequence (SEQ ID NO: 82) consisting of the amino acids at positions 44 to 57, in the amino acid sequence of SEQ ID NO: 28 (full-length PHB2 polypeptide). Accordingly, preferred examples of the peptides of the present invention include a peptide comprising an amino acid sequence selected from the group consisting of:
(a) an amino acid sequence selected from the group consisting of SEQ ID NOs: 1 and 36 to 41 (PHB2 sequence-derived peptides Nos. 1 and 36 to 41);
(b) an amino acid sequence selected from the group consisting of SEQ ID NOs: 5 and 47 to 53 (PHB2 sequence-derived peptides Nos. 5 and 47 to 53); and
(c) an amino acid sequence selected from the group consisting of SEQ ID NOs: 82 and 83 (PHB2 sequence-derived peptides Nos. 82 and 83).

However, the peptides of the present invention are not limited thereto, and the amino acid sequences constituting the peptides are not particularly limited as long as the peptides comprise the BIG3 polypeptide-binding site in a PHB2 polypeptide and have the activity to inhibit the binding between the PHB2 polypeptide and a BIG3 polypeptide.

In general, it is known that one or more amino acid modifications in a peptide have no effect on the function of the peptide. Indeed, it is known that a peptide having an amino acid sequence in which one or more amino acid residues are modified by substitution, deletion, insertion, and/or addition retains the biological activity of the original peptide (Mark et al., Proc Natl Acad Sci USA 81(18): 5662-6 (1984); Zoller and Smith, Nucleic Acids Res 10(20): 6487-500 (1982); and Dalbadie-McFarland et al., Proc Natl Acad Sci USA 79(21): 6409-13 (1982)). The peptides of the present invention may comprise a substitution or deletion of amino acid residues, for example, at positions other than the BIG3 polypeptide-binding site, in a PHB2-derived amino acid sequence and may have an insertion or addition of amino acid residues at positions which have no effect on the binding to a BIG3 polypeptide, as long as they comprise the BIG3 polypeptide-binding site in a PHB2 polypeptide and have the activity to inhibit the binding between the PHB2 polypeptide and the BIG3 polypeptide. Actually, it is shown in Examples of the present specification that peptides having an amino acid sequence in which an amino acid residue(s) other than the BIG3 polypeptide-binding site in a PHB2 polypeptide has/have been substituted with other amino acid residue(s) also retain the biological activity equal to that of peptides which do not have such substitutions. Accordingly, the peptides of the present invention encompass peptides which comprise an amino acid sequence selected from the group consisting of (a′) and (b′) below and have an activity to inhibit the binding between a PHB2 polypeptide and a BIG3 polypeptide:
(a′) an amino acid sequence in which one, two, or several amino acid residues located at positions other than those corresponding to glycine at positions 15 and 18 in the amino acid sequence of SEQ ID NO: 28 (full-length PHB2 polypeptide) are substituted with other amino acid residues, in an amino acid sequence selected from the group consisting of SEQ ID NOs: 1 and 36 to 41 (PHB2 sequence-derived peptides Nos. 1 and 36 to 41); and
(b′) an amino acid sequence in which one, two, or several amino acid residues located at positions other than that corresponding to aspartic acid at position 82 in the amino acid sequence of SEQ ID NO: 28 (full-length PHB2 polypeptide) are substituted with other amino acid residues, in an amino acid sequence selected from the group consisting of SEQ ID NOs: 5 and 47 to 53 (PHB2 sequence-derived peptides Nos. 5 and 47 to 53).

In the above (a′) and (b′), amino acid residues substituted can be any amino acid residues as long as the resulting peptide maintains the ability to inhibit the binding between a PHB2 polypeptide and a BIG3 polypeptide. Furthermore, which amino acid residue is substituted can be determined by predicting amino acid residues not involved in the binding to a BIG3 polypeptide by using, for example, a calculation method such as PSIVER. The number of amino acid residues substituted are also not particularly limited as long as the resulting peptide maintains the ability to inhibit the binding between a PHB2 polypeptide and a BIG3 polypeptide, and one, two, or several amino acid residues can be substituted. “Several” preferably refers to six, five, four, or three.

In general, it is recognized that a substitution with another amino acid residue which conserves the amino acid side chain characteristics of the original amino acid residue tends to have no effect on the function of the original peptide. Such a substitution is often called a “conservative substitution” or “conservative modification”. Accordingly, the substitutions in the above (a′) and (b′) are preferably performed by conservative substitutions.

Tables of conservative substitutions presenting functionally similar amino acids are well known in the art. Examples of amino acid side chain characteristics that are desirable to conserve include, for example, hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). In addition, the following eight groups each contain amino acids that are accepted in the art as conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M) (see, e.g., Creighton, Proteins 1984).

However, the substitutions in the above-mentioned (a′) and (b′) are not limited thereto, and they may be non-conservative substitutions as long as the peptides maintain the activity to inhibit the binding between a PHB2 polypeptide and a BIG3 polypeptide.

The peptides of the present invention can comprise amino acid residues other than the BIG3 polypeptide-binding site in a PHB2 polypeptide as long as they maintain the activity to inhibit the binding between a PHB2 polypeptide and a BIG3 polypeptide. For example, a fragment of PHB2 polypeptide comprising the BIG3 polypeptide-binding site in a PHB2 polypeptide is preferred as a peptide of the present invention. Accordingly, preferred examples of the peptides of the present invention include PHB2 polypeptides (SEQ ID NOs: 1 and 36 to 41 (PHB2 sequence-derived peptides Nos. 1 and 36 to 41)) comprising glycine at positions 15 and 18 and their surrounding sequence in the amino acid sequence of SEQ ID NO: 28 (full-length PHB2 polypeptide) and PHB2 polypeptides (SEQ ID NOs: 5 and 47 to 53 (PHB2 sequence-derived peptides Nos. 5 and 47 to 53)) comprising aspartic acid at position 82 and its surrounding sequence in the amino acid sequence of SEQ ID NO: 28 (full-length PHB2 polypeptide). Other preferred examples of the peptides of the present invention include PHB2 polypeptides (SEQ ID NOs: 82 and 83 (PHB2 sequence-derived peptides Nos. 82 and 83)) comprising amino acids at positions 44 to 57 and their surrounding sequence in the amino acid sequence of SEQ ID NO: 28 (full-length PHB2 polypeptide).

The following peptides can be exemplified as the PHB2 peptides of the present invention: peptides comprising amino acid sequences of SEQ ID NOs: 1, 5, 36 to 41, 47 to 53, 82, and 83 (PHB2 sequence-derived peptides Nos. 1, 5, 36 to 41, 47 to 53, 82, and 83), which peptides are composed of, for example, 30 amino acid residues or 20 amino acid residues, typically 19 amino acid residues, preferably 18 amino acid residues, and more preferably 17 amino acid residues or less. The following peptides can be shown as such peptides: peptides comprising an amino acid sequence selected from the amino acid sequences (9 residues) of SEQ ID NOs: 1, 5, 36 to 41, 47 to 53, 82, and 83 (PHB2 sequence-derived peptides Nos. 1, 5, 36 to 41, 47 to 53, 82, and 83) and an amino acid sequence selected from the full-length amino acid sequence constituting a PHB2 polypeptide, which peptides are composed of 30 amino acid residues or 20 amino acid residues, typically 19 amino acid residues, preferably 18 amino acid residues, and more preferably 17 amino acid residues or less.

In a preferred embodiment of the present invention, an amino acid to be added to the amino acid sequences of SEQ ID NOs: 1, 5, 36 to 41, 47 to 53, 82, and 83 (PHB2 sequence-derived peptides Nos. 1, 5, 36 to 41, 47 to 53, 82, and 83) can be zero (that is, the amino acid sequences consisting of SEQ ID NOs: 1, 5, 36 to 41, 47 to 53, 82, and 83 (PHB2 sequence-derived peptides Nos. 1, 5, 36 to 41, 47 to 53, 82, and 83)) or can be one, two or more consecutive amino acid sequences selected from the full-length amino acid sequence constituting a PHB2 polypeptide (SEQ ID NO: 28 (full-length PHB2 polypeptide)). The amino acid sequences of SEQ ID NOs: 1, 5, 36 to 41, 47 to 53, 82, and 83 (PHB2 sequence-derived peptides Nos. 1, 5, 36 to 41, 47 to 53, 82, and 83) are those comprising glycine at position 15, glycine at position 18, aspartic acid at position 82, or the amino acid sequence consisting of the amino acids at positions 44 to 57 in the full-length amino acid sequence constituting a PHB2 polypeptide (SEQ ID NO: 28 (full-length PHB2 polypeptide)). Accordingly, in a preferred embodiment of the present invention, an amino acid residue or amino acid sequence to be added to SEQ ID NOs: 1, 5, 36 to 41, 47 to 53, 82, and 83 (PHB2 sequence-derived peptides Nos. 1, 5, 36 to 41, 47 to 53, 82, and 83) can be selected from amino acid sequences neighboring glycine at position 15, glycine at position 18, aspartic acid at position 82, or the amino acid sequence consisting of the amino acids at positions 44 to 57 in the amino acid sequence of SEQ ID NO: 28 (full-length PHB2 polypeptide)).

It is desirable that the peptides of the present invention have either or both of the following properties (i) and (ii), in addition to the activity to inhibit the binding between a PHB2 polypeptide and a BIG3 polypeptide:
(i) promoting the nuclear import of a PHB2 polypeptide in an estrogen receptor-positive cell which expresses a BIG3 polypeptide; and
(ii) promoting the binding between an estrogen receptor present in the nucleus and/or on cell membrane to a PHB2 polypeptide in an estrogen receptor-positive cell which expresses a BIG3 polypeptide.

By having either or both of the above properties (i) and (ii), the peptides of the present invention suppress activation of estrogen receptors in BIG3-expressing cells, thereby leading to suppression of growth of estrogen receptor-positive cells. Both the above properties (i) and (ii) of PHB2 peptides can be evaluated according to methods known to one skilled in the art.

A PHB2 polypeptide is known as an estrogen receptor-selective coregulator and suppresses transcriptional activation of estrogen receptors by interaction with them (Kasashima K, J Biol Chem 281(47): 36401-10 (2006)). On the other hand, a BIG3 polypeptide binds to a PHB2 polypeptide to block the nuclear import of a PHB2 polypeptide, thereby inhibiting the interaction between the PHB2 polypeptide and an estrogen receptor in the nucleus. Furthermore, a BIG3 polypeptide blocks the binding between an estrogen receptor present on the cell membrane and a PHB2 polypeptide. As a result of these functions, in cells overexpressing a BIG3 polypeptide, suppression of estrogen receptor activation by the PHB2 polypeptide does not sufficiently work, and enhanced cell growth is induced.

The peptides of the present invention have the feature of restoring the PHB2 polypeptide's function of suppressing activation of estrogen receptors, the function having been inhibited by the binding to a BIG3 polypeptide, by competitively inhibiting the binding between the BIG3 polypeptide and the PHB2 polypeptide. On the other hand, a PHB2 polypeptide suppresses activation of estrogen receptors through the binding to them. Accordingly, it is desirable that the peptides of the present invention suppress the binding between a BIG3 polypeptide and a PHB2 polypeptide but do not block the binding between an estrogen receptor and the PHB2 polypeptide, and thus do not block the suppression of estrogen receptor activation by the PHB2 polypeptide. As described above, a fragment of PHB2 polypeptide comprising the BIG3 polypeptide-binding site is suitable as a peptide of the present invention; however, a peptide close to the full-length of a PHB2 polypeptide is likely to block the binding between an endogenous PHB2 polypeptide and an estrogen receptor, thereby blocking the suppression of estrogen receptor activation by the endogenous PHB2 polypeptide. Thus, a partial amino acid sequence of PHB2 polypeptide comprised in the peptides of the present invention is preferably 100 residues or less, more preferably 80 residues or less, and even more preferably 70 residues or less. In a more preferred embodiment, a partial amino acid sequence of PHB2 polypeptide comprised in the peptides of the present invention is 50 residues or less, 40 residues or less, 30 residues or less, 25 residues or less, or 20 residues or less. Since the estrogen receptor-binding site in PHB2 is a site consisting of the amino acids at positions 175 to 198 in the amino acid sequence of SEQ ID NO: 28 (full-length PHB2 polypeptide), the peptides of the present invention preferably do not comprise this site and in this case, a partial amino acid sequence of PHB2 polypeptide comprised in the peptides of the present invention is desirably 100 residues or less, more preferably 80 residues or less, and even more preferably 70 residues or less, excluding the sequence of amino acids at positions 175 to 198. In a more preferred embodiment, a partial amino acid sequence of PHB2 polypeptide comprised in the peptides of the present invention is 50 residues or less, 40 residues or less, 30 residues or less, 25 residues or less, or 20 residues or less, excluding the sequence of amino acids at positions 175 to 198.

Furthermore, the peptides of the present invention may comprise additional amino acid sequences other than the amino acid sequence derived from a PHB2 polypeptide as long as they maintain the activity to inhibit the binding between a BIG3 polypeptide and a PHB2 polypeptide and do not block the suppression of estrogen receptor activation by the PHB2 polypeptide. Also in this case, it is desirable that the additional amino acid sequences do not block the binding between an endogenous PHB2 polypeptide and an estrogen receptor. Thus, the peptides of the present invention are preferably a peptide of 100 residues or less, 80 residues or less, or 70 residues or less. In a more preferred embodiment, the peptides of the present invention are peptides of 50 residues or less, 40 residues or less, or 30 residues or less. Preferred examples of amino acid sequences comprised in the peptides of the present invention include, but are not limited to, amino acid sequences constituting cell-permeable peptides described later and linker sequences for coupling other substances.

Moreover, the peptides of the present invention may be modified with other substances. Herein, the term “modified” used in the context of a peptide means that another substance(s) is/are directly or indirectly coupled to a peptide. Other substances that modify the peptides of the present invention include, but are not limited to, for example, peptides, lipids, saccharides, and natural or synthetic polymers. The peptides of the present invention can have any modifications as long as they maintain the activity to inhibit the binding between a BIG3 polypeptide and a PHB2 polypeptide. Furthermore, the peptides of the present invention may be conferred additional functions by modifications. Examples of the additional functions include, but are not limited to, targeting property, stability, and cell membrane permeability.

Preferred examples of modifications in the present invention include introduction of a cell membrane permeable substance. Intracellular structure is usually cut off from the outside world by the cell membrane. Thus, it is difficult to efficiently introduce an extracellular substance into a cell. However, a certain type of substance has cell membrane permeability and can be introduced into a cell without being cut off by the cell membrane. It is possible to confer cell membrane permeability to a substance with no cell membrane permeability by modifying the substance with such a substance having cell membrane permeability (cell membrane permeable substance). Accordingly, the peptides of the present invention can be efficiently introduced into cells by modifying the peptide of the present invention with a cell membrane permeable substance(s). Furthermore, as used herein, “cell membrane permeability” refers to the property of being able to permeate the cell membrane of mammals and enter the cytoplasm. Moreover, a “cell membrane permeable substance” refers to a substance having “cell membrane permeability”.

Examples of the cell membrane permeable substance include, but are not limited to, membrane fusogenic liposomes and cell membrane permeable peptides. For example, membrane fusogenic liposomes are fused to cell membrane to release their contents into a cell. Membrane fusogenic liposomes can be prepared, for example, by modifying the surface of liposomes with a substance having membrane fusogenicity. Examples of membrane fusogenic liposomes include pH-sensitive liposomes (Yuba E, et al., J. Control. Release, 149, 72-80 (2011)), Sendai virus membrane fusogenic liposomes (WO 97/016171), and liposomes modified with cell membrane permeable peptides. The peptides of the present invention may be encapsulated into a membrane fusogenic liposome to efficiently introduce the peptides into a cell. In the present invention, encapsulation of a peptide into a membrane fusogenic liposome is also encompassed in “modification” of a peptide.

Various natural or artificially synthesized peptides have so far been reported as cell membrane permeable peptides (Joliot A. & Prochiantz A., Nat Cell Biol. 2004; 6:189-96). Examples of cell membrane permeable peptides include the following peptides, but are not limited thereto.

Polyarginine (Matsushita et al., J. Neurosci.;
21(16), 6000-7 (2003));
(SEQ ID NO: 90)
Tat/RKKRRQRRR
(Frankel et al., Cell 55(6), 1189-93
(1988)., Green &
Loewenstein Cell 55, 1179-88 (1988));
(SEQ ID NO: 103)
Penetratin/RQIKIWFQNRRMKWKK
(Derossi et al., J. Biol. Chem.
269(14), 10444-50 (1994));
(SEQ ID NO: 91)
Buforin II/TRSSRAGLQFPVGRVHRLLRK
(Park et al., Proc. Natl Acad.
Sci. USA 97(15), 8245-50 (2000));
(SEQ ID NO: 92)
Transportan/GWTLNSAGYLLGKINLKALAALAKKIL
(Pooga et al.,
FASEB J. 12(1), 67-77 (1998));
(SEQ ID NO: 93)
MAP (Model Amphipathic Peptide)/KLALKLALKALKAALKLA
(Oehlke et
al., Biochim. Biophys. Acta. 1414(1-2),
127-39 (1998));
(SEQ ID NO: 94)
K-FGF/AAVALLPAVLLALLAP
(Lin et al., J. Biol. Chem. 270(24),
14255-8 (1995));
(SEQ ID NO: 95)
Ku70/VPMLK
(Sawada et al., Nature Cell Biol. 5(4),
352-7 (2003));
(SEQ ID NO: 96)
Ku70/PMLKE
(Sawada et al., Nature Cell Biol. 5(4),
352-7 (2003));
(SEQ ID NO: 97)
Prion/MANLGYWLLALFVTMWTDVGLCKKRPKP
(Lundberg et al.,
Biochem. Biophys. Res. Commun. 299(1),
85-90 (2002));
(SEQ ID NO: 98)
pVEC/LLIILRRRIRKQAHAHSK
(Elmquist et al., Exp. Cell Res. 269(2),
237-44 (2001));
(SEQ ID NO: 99)
Pep-1/KETWWETWWTEWSQPKKKRKV
(Morris et al., Nature Biotechnol.
19(2), 1173-6 (2001));
(SEQ ID NO: 100)
SynB1/RGGRLSYSRRRFSTSTGR
(Rousselle et al., Mol. Pharmacol. 57(4),
679-86 (2000));
(SEQ ID NO: 101)
Pep-7/SDLWEMMMVSLACQY
(Gao et al., Bioorg. Med. Chem. 10(12),
4057-65 (2002));
and
(SEQ ID NO: 102)
HN-1/TSPLNIHNGQKL;
(Hong & Clayman Cancer Res. 60(23), 6551-6
(2000)).

The above-mentioned polyarginine may be composed of any number of arginine residues. For example, polyarginine may be composed of 5 to 20 arginine residues. The number of arginine residues constituting polyarginine is not particularly limited as long as it does not block the activity of the peptide to inhibit the binding between a BIG3 polypeptide and a PHB2 polypeptide.

Furthermore, it is known in the art to introduce various particularly useful amino acid mimetics or unnatural amino acids (for example, by substitution, addition, or insertion) in order to increase in vivo stability of peptides. Examples of amino acid mimetics or unnatural amino acids to be introduced include, but are not limited to, β-amino acids, D-amino acids, and N-methyl amino acids. Accordingly, such amino acid mimetics or unnatural amino acids can be introduced into the peptides of the present invention to increase in vivo stability. Moreover, azapeptides in which «-carbons of amino acids have been substituted with an amino group and techniques for substituting amide bonds in peptides with their equivalents (such as esters, sulfonamides, and alkene isosteres) are also known in the art. Stability of peptides can be confirmed using, for example, peptidases and various biological media such as human plasma and serum (see, for example, Coos Verhoef et al. Eur. J. Drug Metab. Pharmacokin. 11(4): 291-302 (1986)).

Accordingly, the present invention provides peptides comprising the BIG3 polypeptide-binding site in a PHB2 polypeptide, which inhibit the binding between the PHB2 polypeptide and a BIG3 polypeptide and comprise at least one amino acid mimetic or unnatural amino acid (for example, β-amino acid, D-amino acid, and N-methyl amino acid). In a particular embodiment, the peptides of the present invention comprise an amino acid sequence in which one, two, or several amino acids are substituted with corresponding amino acid mimetics or unnatural amino acids (for example, β-amino acids, D-amino acids, and N-methyl amino acids) in an amino acid sequence selected from the group consisting of SEQ ID NOs: 1, 5, 36 to 41, 47 to 53, 82, and 83 (PHB2 sequence-derived peptides Nos. 1, 5, 36 to 41, 47 to 53, 82, and 83).

Moreover, the present invention also provides peptides comprising the BIG3 polypeptide-binding site in a PHB2 polypeptide, which inhibit the binding between the PHB2 polypeptide and a BIG3 polypeptide and in which at least one amide bond has been replaced with its equivalent (for example, an ester, sulfonamide, and alkene isostere).

### Cyclic and Cross-Linked Peptides

In a particular embodiment, the peptides of the present invention may be cyclized and their stability can be improved by cyclization. Methods of introducing a cyclic structure into a peptide of the present invention are well known, and for example, a peptide can be cyclized by adding cysteine to the N- and C-termini of a linear peptide and allowing to form a disulfide bond between these cysteines. Herein, such a structure in which side chains of two (a pair of) amino acid residues in an amino acid sequence constituting a peptide are cross-linked (stapled) can be called a “stapling structure”, and a cross-linked peptide into which one or more stapling structures have been introduced is also referred to as a “stapled peptide”. Positions of amino acid residues forming such an intramolecular crosslink are not limited to the N- and C-termini of the original linear peptide, and amino acid residues present in the original linear peptide may form an intramolecular crosslink, or amino acid residues introduced into the original linear peptide (by, for example, substitution, addition, or insertion) may form an intramolecular crosslink. Amino acid residues forming an intramolecular crosslink are not limited to natural amino acids and may be amino acid mimetics or unnatural amino acids as described above. Moreover, methods of cross-linking a peptide are not limited to disulfide bond formation, and also include cross-linking of cysteine residues through fluorobenzene (for example, by using hexafluorobenzene or decafluorobiphenyl), thioether bond formation, ester bond formation, and a technique for stapling hydrocarbons such as ring-closing olefin metathesis (described in, for example, WO 2017/126461).

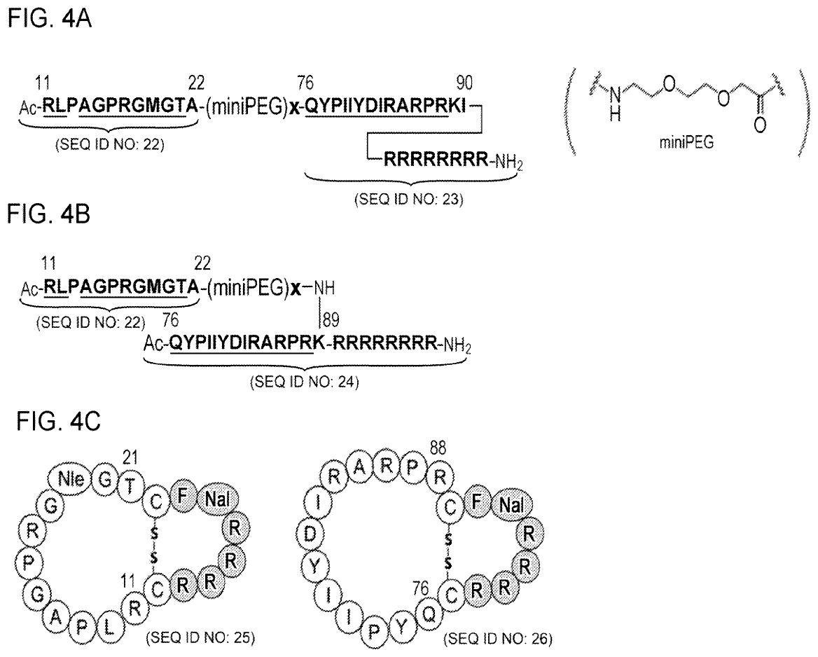

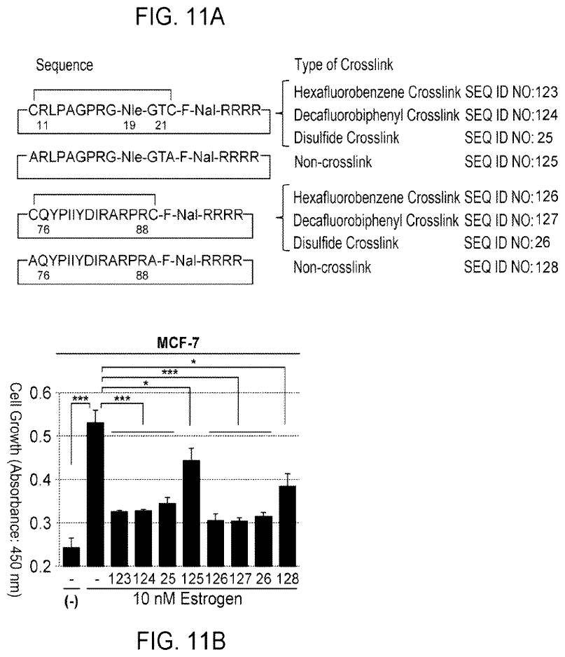

Furthermore, methods of cyclizing a peptide are not limited to methods of forming an intramolecular crosslink as mentioned above, and also include formation of an amide bond between the C-terminal and N-terminal amino acid residues of a peptide. The peptides thus cyclized by various methods are herein referred to as cyclic peptides, and include both cyclic peptides comprising an intramolecular crosslink (i.e., cross-linked peptides; for example, SEQ ID NOs: 25, 26, 123, 124, 126, and 127 depicted in FIGS. 4 and 11) and those not comprising an intramolecular crosslink (for example, SEQ ID NOs: 125 and 128 depicted in FIG. 11).

Accordingly, the present invention provides cyclic peptides comprising the BIG3 polypeptide-binding site in a PHB2 polypeptide, which inhibit the binding between the PHB2 polypeptide and a BIG3 polypeptide and have been cyclized by at least one intramolecular bond. Examples of the intramolecular bond include, but are not limited to, a disulfide bond, crosslink between cysteine residues through fluorobenzene (for example, by using hexafluorobenzene or decafluorobiphenyl), thioether bond, ester bond, thioester bond, bond by a hydrocarbon chain (for example, olefin and aryl), bond by a heterocycle (for example, triazole, oxazole, and thiazole) and amido bond, and combinations thereof.

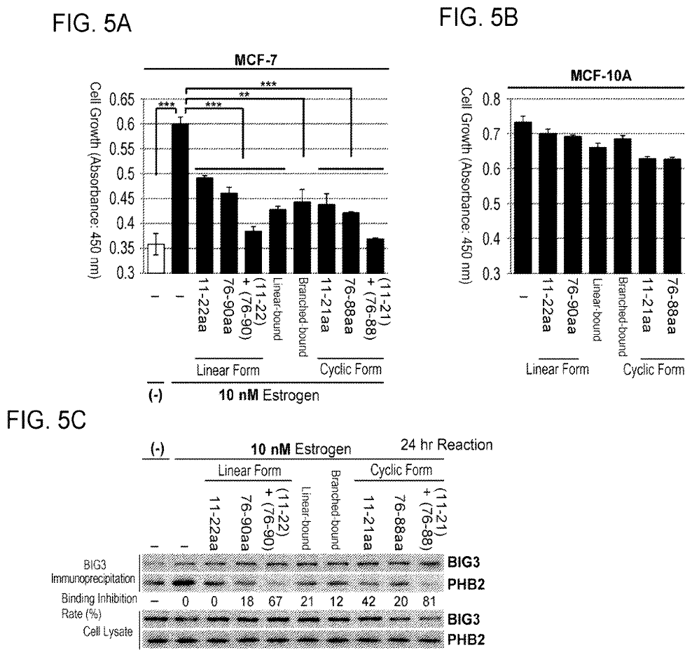

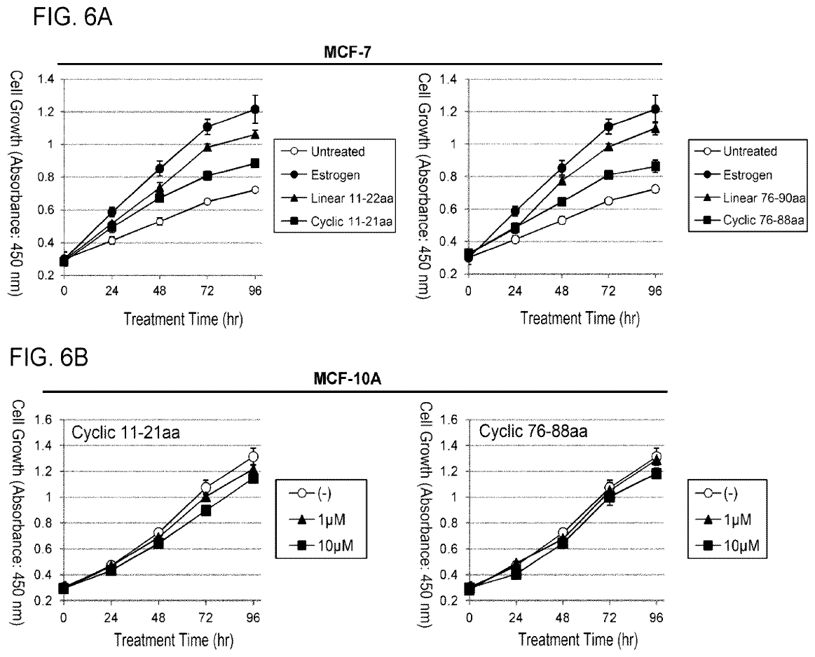

Such an intramolecular bond may be formed by amino acid residues at both ends of the original linear peptide of a cyclic peptide, or may be formed by amino acid residues in the linear peptide. Moreover, the intramolecular bond may be formed by amino acid residues in an amino acid sequence derived from a PHB2 polypeptide, or may be formed by amino acid residues introduced into the amino acid sequence (by, for example, substitution, addition, or insertion). Preferred examples of such cyclic peptides of the present invention include cyclic PHB2 peptides of 11-21aa and 76-88aa (cyclic peptides respectively consisting of SEQ ID NO: 25, 106 to 108, 115 to 117, 123, or 124; and SEQ ID NO: 26, 110 to 112, 119 to 121, 126, or 127). These exemplary cyclic peptides are prepared by adding two cysteine residues that form an intramolecular bond (intramolecular crosslink) (SEQ ID NOs: 25, 26, 106 to 108, 110 to 112, 115 to 117, 119 to 121, 123, 124, 126, and 127); adding or substituting unnatural amino acids (SEQ ID NOs: 25, 26, 106 to 108, 115 to 117, 123, 124, 126, and 127); and adding consecutive multiple arginine residues (SEQ ID NOs: 25, 26, 106 to 108, 110 to 112, 123, 124, 126, and 127), in the linear peptides of PHB2 sequence-derived 11-21aa and 76-88aa with the objective of stabilization of the structure (and increase in the activity and improvement of protease resistance accompanied thereby) and improvement of membrane permeability. Such peptides have been cyclized by formation of an intramolecular crosslink between the two cysteine residues introduced (SEQ ID NOs: 25, 26, 106 to 108, 110 to 112, 115 to 117, 119 to 121, 123, 124, 126, and 127) and by formation of an amido bond between the C-terminal and N-terminal amino acid residues added (SEQ ID NOs: 25, 26, 123, 124, 126, and 127) (FIGS. 4C, 10A, and 11A). These exemplary cyclic peptides (in particular, cyclic and cross-linked peptides) showed an enhanced growth suppressive effect as compared to the original linear peptides (FIGS. 5A, 10C, and 11B) and the suppressive effect was shown to last for a long time (FIG. 6A).

The present invention also relates to methods of producing a cyclic peptide, the method comprising:
(a) providing a linear peptide comprising the BIG3 polypeptide-binding site in a PHB2 polypeptide, which inhibits the binding between the PHB2 polypeptide and a BIG3 polypeptide; and
(b) allowing to form at least one intramolecular bond in the linear peptide, thereby cyclizing the linear peptide.

The intramolecular bond is optionally selected from the group consisting of a disulfide bond, crosslink between cysteine residues through fluorobenzene (for example, by using hexafluorobenzene or decafluorobiphenyl), thioether bond, ester bond, thioester bond, bond by a hydrocarbon chain (for example, olefin and aryl), bond by a heterocycle (for example, triazole, oxazole, and thiazole) and amido bond, and combinations thereof.

The above methods optionally comprise introducing at least one selected from the group consisting of cysteine residues, an amino acid mimetic(s) or unnatural amino acid(s), and consecutive multiple arginine residues into the linear peptide (by, for example, substitution, addition, or insertion).

The peptides of the present invention have the feature of having the activity to inhibit the binding between a PHB2 polypeptide and a BIG3 polypeptide. Whether a peptide produced has the activity to inhibit the binding between a PHB2 polypeptide and a BIG3 polypeptide or not can be confirmed by comparing the binding level between the PHB2 polypeptide and the BIG3 polypeptide in the presence and absence of the peptide. That is, when the binding level in the presence of a peptide is lower than that in the absence of the peptide, the peptide can be judged to have “the activity to inhibit the binding between a PHB2 polypeptide and a BIG3 polypeptide”.

The binding level between a PHB2 polypeptide and a BIG3 polypeptide can be measured using various known methods. For example, immunoprecipitation using an anti-PHB2 antibody or an anti-BIG3 antibody, affinity chromatography, a biosensor using surface plasmon resonance phenomenon, and such can be used.

As a specific method, for example, a PHB2 polypeptide and a BIG3 polypeptide are incubated in the presence and absence of a test peptide. The reaction solution is then immunoprecipitated with an anti-PHB2 antibody or an anti-BIG3 antibody, and the immunoprecipitate is subjected to Western blot analysis. The binding level between the PHB2 polypeptide and the BIG3 polypeptide can be confirmed by detecting at least either one of the BIG3 polypeptide level immunoprecipitated with the anti-PHB2 antibody or the PHB2 polypeptide level immunoprecipitated with the anti-BIG3 antibody. The PHB2 polypeptide and BIG3 polypeptide used here can be prepared by a known genetic engineering technique. Furthermore, lysates of cells producing these polypeptides can be used. The cell lines as described in Examples of the present specification can be utilized as cells producing these polypeptides.

Alternatively, the methods as described in Examples of the present specification can also be used. Specifically, estrogen receptor-positive cells are cultured in the presence and absence of a test peptide. The cells are then lysed with an appropriate lysis buffer, and the cell lysate may be used to perform immunoprecipitation and Western blot analysis in the same manner as above.

A peptide for which “the activity to inhibit the binding between a PHB2 polypeptide and a BIG3 polypeptide” has been confirmed by any of the above methods is judged to be a peptide having “the activity to inhibit the binding between a PHB2 polypeptide and a BIG3 polypeptide”.

Moreover, the peptides of the present invention may have either or both of the following (i) and (ii) as preferred properties:
(i) promoting the nuclear import of a PHB2 polypeptide in an estrogen receptor-positive cell which expresses a BIG3 polypeptide; and
(ii) promoting the binding between an estrogen receptor present in the nucleus and/or on cell membrane and a PHB2 polypeptide in an estrogen receptor-positive cell which expresses a BIG3 polypeptide.

Whether a peptide of the present invention has the above properties or not can be confirmed by comparing (i) the nuclear import level of a PHB2 polypeptide; and/or (ii) the binding level between an estrogen receptor and a PHB2 polypeptide, in the presence and absence of the peptide of the present invention. That is, when the level in the presence of the peptide of the present invention is higher as compared to that in the absence of the peptide, the peptide can be judged to have the above-mentioned properties (i) and/or (ii).

Methods well known to one skilled in the art can be used as examples of specific methods for judging the presence or absence of the above-mentioned properties (i) and/or (ii). Specifically, when examining the above property (i), estrogen receptor-positive cells are stimulated with estradiol for 24 hours with or without the addition of a peptide of the present invention. The cells are then fractionated by specific gravity centrifugation, and PHB2 polypeptides present in the nuclear fraction are detected by Western blot analysis and such. When the level of PHB2 polypeptide detected in the nuclear fraction increases in the case where the peptide of the present invention is added as compared to the case where the peptide is not added, the peptide of the present invention is judged to have the above-mentioned property (i). Moreover, the level of PHB2 polypeptide present in the nucleus can be detected by immunocytochemical staining.

When examining the above-mentioned property (ii), estrogen receptor-positive cells are stimulated with estradiol for 24 hours with or without the addition of a peptide of the present invention. The cells are then fractionated by specific gravity centrifugation, the cytosolic fraction and the nuclear fraction are immunoprecipitated with an anti-estrogen receptor antibody or an anti-PHB2 antibody, and the immunoprecipitate is subjected to Western blot analysis. As a result, when the binding level between an estrogen receptor and a PHB2 polypeptide in the cytosolic fraction and/or the nuclear fraction increases in the case where the peptide of the present invention is added as compared to the case where the peptide is not added, the peptide of the present invention is judged to have the above-mentioned property (ii).

The peptides of the present invention can be produced using methods well known to one skilled in the art. For example, the peptides of the present invention can be obtained by chemical synthesis based on their amino acid sequences. Methods for chemical synthesis of a peptide are known and one skilled in the art can chemically synthesize the peptide of the present invention based on amino acid sequence selected as the peptide of the present invention. Chemical synthesis methods of peptide are described, for example, in the documents below:
(i) Peptide Synthesis, Interscience, New York, 1966;
(ii) The Proteins, Vol. 2, Academic Press, New York, 1976;
(iii) “Peptide Synthesis” (in Japanese), Maruzen Co., 1975;
(iv) “Basics and Experiment of Peptide Synthesis” (in Japanese), Maruzen Co., 1985;
(v) “Development of Pharmaceuticals” (in Japanese), Continued Vol. 14 (peptide synthesis), Hirokawa, 1991;
(vi) WO99/67288; and
(vii) Barany G. & Merrifield R. B., Peptides Vol. 2, Solid Phase Peptide Synthesis, Academic Press, New York, 1980, 100-118.

Alternatively, the peptides of the present invention can be obtained by genetic engineering methods (e.g., Morrison J, J Bacteriology, 132(1): 349-51 (1977); Clark-Curtiss & Curtiss, Methods in Enzymology (eds. Wu et al.), 101:347-62 (1983)). For example, a polynucleotide encoding a peptide of the present invention is inserted into an appropriate expression vector, and the vector is introduced into appropriate host cells to prepare transformed cells. The transformed cells are then cultured to produce the peptide of the present invention, and the cell extract is prepared. Standard techniques for purifying proteins can be used to purify the peptide of the present invention from the cell extract. The peptide of the present invention can be purified by, for example, appropriately selecting and combining column chromatography, filter filtration, ultrafiltration, salting-out, solvent precipitation, solvent extraction, distillation, immunoprecipitation, SDS-polyacrylamide gel electrophoresis, isoelectric focusing, dialysis, and recrystallization. Furthermore, the peptides of the present invention can be synthesized by in vitro translation system in which elements necessary to synthesize protein have been reconstructed in vitro.

When using genetic engineering techniques, the peptides of the present invention can also be expressed as a fusion protein with other peptide. A polynucleotide encoding a peptide of the present invention is ligated in frame with a polynucleotide encoding other peptide and inserted into an appropriate expression vector, and the vector is introduced into appropriate host cells to prepare transformed cells. The host cells are then cultured to allow production of a fusion protein of the peptide of the present invention and the other peptide, and its cell extract is prepared. The purification of fusion protein from the cell extract can be performed by, for example, capturing the fusion protein by affinity chromatography using a column to which a substance having affinity to the fusion protein has been coupled. Moreover, if the peptide of the present invention has been coupled to another peptide through a linker sequence which can be cleaved by an enzyme such as peptidase, protease, and proteasome, the peptide of the present invention can be separated from the column by treating the fusion protein captured by the column with such an enzyme. Examples of other peptides which can be used to form fusion proteins include the following peptides, but are not limited thereto:
FLAG (Hopp et al., Bio/Technology 6, 1204-10 (1988));
6×His or 10×His consisting of histidine (His) residues;
Influenza Hemagglutinin (HA);
Human c-myc fragment, VSV-GP fragment; p18 HIV fragment;
T7 tag; HSV tag;
E tag; SV40T antigen fragment;
lck tag;
α-Tubulin fragment;
B tag;
Protein C fragment;
GST (Glutathione-S-transferase);
HA (Influenza Hemagglutinin);
Immunoglobulin constant region;
β-Galactosidase; and
MBP (Maltose-binding protein).

### 2. Polynucleotides Encoding the Peptides of the Present Invention, Vectors, and Host Cells

The present invention also provides polynucleotides encoding the peptide of the present invention. Furthermore, the present invention provides vectors comprising the polynucleotide and host cells comprising the vector. Such polynucleotides, vectors, and host cells can be used to produce the peptides of the present invention.

The polynucleotides of the present invention can be produced by methods known to one skilled in the art. For example, the polynucleotides of the present invention can be synthesized using solid-phase techniques as described in Beaucage S L & Iyer R P, Tetrahedron, 48:2223-311 (1992); Matthes et al., EMBO J, 3(4): 801-5 (1984). Moreover, the polynucleotides of the present invention can be prepared using genetic engineering techniques. For example, primers are produced based on a partial nucleotide sequence of a PHB2 gene (SEQ ID NO: 27) encoding an amino acid sequence selected as a peptide of the present invention, and reverse transcription-PCR is performed by using mRNAs extracted from cells expressing a PHB2 polypeptide as a template. Thus, the polynucleotides of the present invention can be amplified.

The polynucleotides of the present invention can be inserted into an appropriate expression vector and the vector is introduced into appropriate host cells to produce the peptide of the present invention in the host cells.

For example, when E. coli is selected as a host cell and a vector is amplified in a large amount in E. coli (for example, JM109, DH5-alpha, HB101 or XL1 Blue), the vector needs to have an “ori” for amplification in E. coli and a marker gene for selection of transformed E. coli (for example, a drug resistance gene selected by a drug such as ampicillin, tetracycline, kanamycin, chloramphenicol). For example, the M13-series vectors, pUC-series vectors, pBR322, pBluescript, pCR-Script and such can be used. When a vector is used for producing a peptide of the present invention, an expression vector is particularly useful. For example, an expression vector for expression in E. coli needs to have the above features for amplification in E. coli. When E. coli such as JM109, DH5-alpha, HB101 or XL1 Blue are used as a host cell, the vector needs to have a promoter, for example, lacZ promoter (Ward et al., Nature, 341(6242): 544-6(1989); FASEB J, 6(7): 2422-7 (1992)), araB promoter (Better et al., Science, 240(4855): 1041-3 (1988)), T7 promoter or the like, that can efficiently express the desired gene in E. coli. Additionally, the vector may contain a signal sequence for polypeptide secretion. An exemplary signal sequence that directs the polypeptide to be secreted to the periplasm of the E. coli is the pelB signal sequence (Lei et al., J Bacteriol, 169(9): 4379-83 (1987)). Means for introducing the vectors into the target host cells include, for example, the calcium chloride method and the electroporation method.

In addition to E. coli, for example, expression vectors derived from mammalian cells (for example, pcDNA3 (Invitrogen) and pEGF-BOS (Mizushima S., Nucleic Acids Res, 18(17): 5322 (1990)), pEF, pCDM8), expression vectors derived from insect cells (for example, “Bac-to-BAC baculovirus expression system” (GIBCO BRL), pBacPAK8), expression vectors derived from plants (e.g., pMH1, pMH2), expression vectors derived from animal viruses (e.g., pHSV, pMV, pAdexLcw), expression vectors derived from retroviruses (e.g., pZIpneo), expression vectors derived from yeast (e.g., “Pichia Expression Kit” (Invitrogen), pNV11, SP-Q01) and expression vectors derived from Bacillus subtilis (e.g., pPL608, pKTH50) can be used.

In order to express the vector in animal cells such as CHO cells, COS cells or NIH3T3 cells, the vector needs to carry a promoter necessary for expression in such cells, for example, the SV40 promoter (Mulligan et al., Nature, 277(5692): 108-14 (1979)), the MMLV-LTR promoter, the EF1-alpha promoter (Mizushima et al., Nucleic Acids Res, 18(17): 5322 (1990)), the CMV promoter and the like, and preferably a marker gene for selecting transformants (for example, a drug resistance gene selected by a drug (e.g., neomycin, G418)). Examples of known vectors with these characteristics include, for example, pMAM, pDR2, pBK-RSV, pBK-CMV, POPRSV and pOP13.

Furthermore, the polynucleotide of the present invention may be inserted into an appropriate vector and the vector is introduced into target cells to produce the peptide of the present invention within the target cells. A peptide of the present invention produced in a target cell inhibits the binding between a PHB2 polypeptide and a BIG3 polypeptide and induces suppression of growth of the target cell. In this case, the vector into which a polynucleotide of the present invention is inserted may be a vector for stably inserting the polynucleotide of the present invention into the genome of the target cell (for example, see Thomas K R & Capecchi M R, Cell, 51(3): 503-12 (1987) for description of cassette vectors for homologous recombination). For example, see Wolff et al., Science, 247:1465-8 (1990); U.S. Pat. Nos. 5,580,895; 5,589,466; 5,804,566; 5,739,118; 5,736,524; 5,679,647; and WO 98/04720.

Moreover, the polynucleotide of the present invention can be inserted into, for example, an expression vector such as a viral vector and a bacterial vector. Examples of the expression vector include a host for an attenuated virus of cowpox, fowlpox, and the like (see, for example, U.S. Pat. No. 4,722,858). Other examples of vectors that can be used include Bacille Calmette Guerin (BCG) (Stover et al., Nature, 351(6326): 456-60 (1991)). Other examples include adenovirus vectors, adeno-associated virus vectors, retrovirus vectors, Salmonella typhi vectors, and attenuated anthrax toxin vectors (Shata et al., Mol Med Today, 6(2): 66-71 (2000); Shedlock et al., J Leukoc Biol, 68(6): 793-806 (2000); and Hipp et al., In Vivo, 14(5): 571-85 (2000)).

Peptides of the present invention encompass peptides in which either or both of the N-terminal and C-terminal amino acid residues have been modified. The types of modifications are not particularly limited, but those that do not affect the affinity for BIG3 are preferred. Examples of preferred modifications include acetylation of the N-terminal amino acid residue, amidation of the C-terminal amino acid residue, addition of tag peptides such as HA-tag and FLAG-tag, and such.

The peptides of the present invention are not limited to those composed of L-amino acids and may be peptides including one or more D-amino acids. The composition ratio of L-amino acids to D-amino acids in the peptides is not particularly limited, and there may be any of the following cases: all amino acid residues may be in L-form (hereinafter referred to as “L-form peptide”); all amino acid residues may be in D-form (hereinafter referred to as “D-form peptide”); or only amino acid residues at a particular position(s) may be in D-form. One preferred embodiment of the peptides of the present invention includes a peptide in which all the amino acid residues have been substituted with D-form amino acid residues in any of the above-mentioned peptides of the present invention. Another preferred embodiment of the peptides of the present invention includes a peptide in which an amino acid residue(s) at a particular position(s) important for the binding to BIG3 has/have been substituted with the corresponding D-form amino acid residue(s). Examples of such a position include positions corresponding to glycine at position 15, glycine at position 18, and aspartic acid at position 82 in the amino acid sequence of SEQ ID NO: 28.

Furthermore, the peptides of the present invention may be retro-inverso forms of any of the above-mentioned peptides of the present invention. A retro-inverso form has an amino acid sequence that is reversed from that of the original peptide, and all amino acid residues are substituted with D-form amino acid residues. More specifically, a retro-inverso form is a D-form peptide having an amino acid sequence that is reversed from that of the original peptide. Therefore, peptides which are retro-inverso forms of any one of the above-mentioned peptides of the present invention are included as preferred embodiments of the peptides of the present invention.

Peptides of the present invention may also be in the form of salts. The form of salts is not particularly limited, but pharmaceutically acceptable salts are preferred. Herein, the “pharmaceutically acceptable salt” refers to a salt that retains the pharmacological and pharmaceutical efficacy and characteristics of a peptide. Preferred examples of salts include salts with alkali metals (lithium, potassium, sodium and such), salts with alkaline-earth metals (calcium, magnesium and such), salts with other metals (copper, iron, zinc, manganese and such), salts with organic bases, salts with amines, salts with organic acids (acetic acid, formic acid, propionic acid, fumaric acid, maleic acid, succinic acid, tartaric acid, citric acid, malic acid, oxalic acid, benzoic acid, methanesulfonic acid, and such), salts with inorganic acids (hydrochloric acid, phosphoric acid, hydrobromic acid, sulfuric acid, nitric acid and such), and such. These salts can be prepared according to known methods.

### 3. Pharmaceutical Compositions Comprising the Peptide or Polynucleotide of the Present Invention and Uses Thereof

The present invention also provides pharmaceutical compositions comprising a peptide or a salt thereof of the present invention or a polynucleotide encoding a peptide of the present invention, along with a pharmaceutically acceptable carrier.

The peptides of the present invention inhibit the binding between a PHB2 polypeptide and a BIG3 polypeptide to induce the suppression of estrogen receptor activation by the PHB2 polypeptide, thereby leading to suppression of growth of estrogen receptor-positive cells. Accordingly, the pharmaceutical compositions of the present invention are useful in either or both of treatment and prophylaxis (prevention) of cell proliferative diseases caused by activation of estrogen receptors. Such cell proliferative diseases include, for example, cancer.

It is known that among cancers, particularly breast cancer is deeply related to activation of estrogen receptors. A BIG3 polypeptide is a novel estrogen receptor activation regulator and is frequently expressed in many breast cancer specimens and breast cancer cells; meanwhile, it has been confirmed that expression of a BIG3 polypeptide is hardly found in normal tissues (Kim J W, Akiyama M, Park J H, et al. Cancer Sci.; 100(8): 1468-78 (2009)). Thus, it is considered that in breast cancer, expression of a BIG3 polypeptide inhibits the function of a PHB2 polypeptide of suppressing activation of estrogen receptors, thereby growth of breast cancer cells is promoted. Accordingly, the pharmaceutical compositions of the present invention are particularly suitable for either or both of treatment and prophylaxis (prevention) of breast cancer. Furthermore, among breast cancers, the pharmaceutical compositions of the present invention are particularly useful in breast cancers that are estrogen receptor-positive and express BIG3 polypeptides. However, the pharmaceutical compositions of the present invention are not limited to use for breast cancer, and they can be used for any cancer as long as it is estrogen receptor-positive and expresses a BIG3 polypeptide. Examples of estrogen receptor-positive cancer besides breast cancer include endometrial cancer, ovarian cancer, prostate cancer (Nelles J L, et al., Expert Rev Endocrinol Metab., 6(3): 437-51 (2011)), and lung cancer (particularly non-small-cell lung cancer) (Stabile L P, et al., Cancer Res., 65(4): 1459-70 (2005); Marquez-Garban D C, et al., Steroids. 72(2): 135-43 (2007)), but are not limited thereto. Cancers to which pharmaceutical compositions of the present invention are applied preferably express BIG3 and PHB2, and estrogen receptor-positive cancers generally express BIG3 and PHB2. Whether a cancer is estrogen receptor-positive can be confirmed by known methods such as ELISA or immunohistochemical staining.

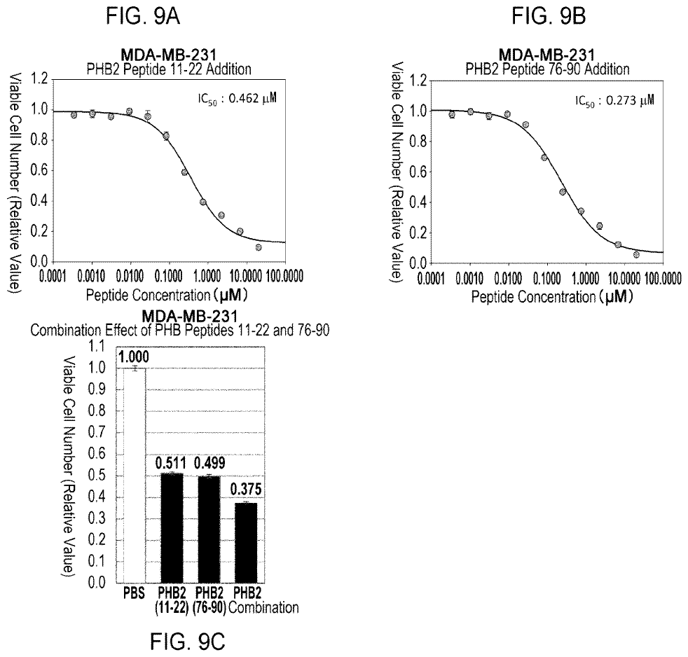

Furthermore, the peptides of the present invention showed excellent cell growth inhibitory effects in triple-negative breast cancer cells, which are estrogen receptor-negative breast cancer cells (FIG. 9). Generally, triple-negative refers to breast cancer cells lacking expression of HER2, estrogen receptors, and progesterone receptors, which are targeted factors in major drug treatments. Therefore, triple-negative breast cancers are normally resistant to drug treatment. Therefore, the pharmaceutical compositions of the present invention can be applied to either or both of treatment and prophylaxis (prevention) of estrogen receptor-negative breast cancer, and are also useful as pharmaceutical compositions for administration to patients with such treatment-resistant breast cancers.

More specifically, the present invention provides pharmaceutical compositions comprising a peptide of the present invention, which are for administration to drug therapy-resistant breast cancer patients. The present invention also relates to peptides of the present invention for use in either or both of treatment and prophylaxis (prevention) of drug therapy-resistant breast cancer patients. Furthermore, the present invention relates to use of the peptides of the present invention in the production of pharmaceutical compositions for either or both of treatment and prophylaxis (prevention) of drug therapy-resistant breast cancer patients. The present invention also provides methods for either or both of treatment and prophylaxis (prevention) of breast cancer which comprise the steps of selecting patients having drug therapy-resistant breast cancer, and administering a peptide of the present invention to the selected patients.

Patients with drug therapy-resistant breast cancer can be identified by observing the therapeutic outcome after common drug therapy. Specifically, when degeneration of the disease focus is not clearly observed by the treatment, one can know that this cancer is treatment-resistant. A condition where enlargement of the disease focus is prevented is included in the degeneration of the disease focus. The above-mentioned triple-negative breast cancer patients are said to have resistance to drug therapies. Triple-negative refers to breast cancers having the features of lacking expression of estrogen receptors and progesterone receptors in addition to HER2. These markers for drug therapy resistance can be evaluated quantitatively by immunostaining and gene expression profiling. For example, the marker status is determined to be negative when the expression level is approximately the same as that of a negative control. For the negative control, treatment-resistant cancer cell lines lacking expression of these markers can be used.

Pharmaceutical compositions of the present invention can be produced using known drug formulation techniques by mixing a peptide or a salt thereof of the present invention with a pharmaceutically acceptable carrier. Herein, “pharmaceutically acceptable carrier” refers to an inactive substance to be used as diluents or solvents for drugs. For the pharmaceutically acceptable carriers to be used in pharmaceutical compositions of the present invention, carriers generally used for pharmaceutical products can be appropriately selected according to the dosage form of the pharmaceutical compositions to be prepared.

The dosage forms of the pharmaceutical compositions of the present invention are not particularly limited, and dosage forms generally used for pharmaceutical products such as liquids, tablets, elixirs, capsules, granules, and powders can be selected appropriately. Furthermore, depending on the selected dosage form, additives such as excipients, stabilizers, suspensions, preservatives, surfactants, solubilizing agents, pH adjusters, and aggregation inhibitors can be added appropriately.

The pharmaceutical compositions of the present invention comprise as an active ingredient a pharmaceutically effective amount of a peptide or a salt thereof of the present invention or a polynucleotide encoding the peptide. A “pharmaceutically effective amount” is an amount sufficient for a pharmaceutical composition of the present invention to accomplish its objective. For example, when a pharmaceutical composition of the present invention is for a pharmaceutical composition for either or both of the treatment and prophylaxis (prevention) of cancer, an example of a pharmaceutically effective amount can be an amount that induces suppression of cancer growth rate, suppression of metastatic potential, prolonged survival time, suppression or delay of cancer development, or alleviation of various clinical symptoms associated with cancer, when administered to a patient. Suppression of cancer growth rate can be, for example, suppression of about 5% or more compared to when the pharmaceutical composition of the present invention is not administered. Preferably, suppression of cancer growth rate can be about 10% or more, 20% or more, 30% or more, 40% or more, 50% or more, 75% or more, 80% or more, 90% or more, or 100% or more.

The pharmaceutically effective amount can be selected appropriately according to the dosage form of the pharmaceutical compositions, dosage interval, age, gender, body weight, and body surface area of subjects for administration, type of disease, and such. Examples of the content of peptides or salts thereof of the present invention in pharmaceutical compositions of the present invention include 0.001 mg to 1000 mg, 0.01 mg to 100 mg, 0.1 mg to 30 mg, or 0.1 mg to 10 mg, but are not limited thereto.

Pharmaceutical compositions of the present invention may optionally include other pharmaceutical agents. Examples of other pharmaceutical agents include anti-inflammatory agents, analgesic agents, antipyretics, other therapeutic agents for cancer, and such. Other therapeutic agents for cancer that may be used for pharmaceutical compositions of the present invention are not particularly limited, but when the pharmaceutical compositions are used for estrogen-positive cancers, examples may include hormone therapy agents such as selective ERα modulators (e.g., tamoxifen and raloxifene), ERα down-regulators (e.g., fulvestrant), aromatase inhibitors, LH-RH agonist formulations, and progesterone formulations. These pharmaceutical agents may also be mixed in the form of prodrugs and pharmaceutically acceptable salts.

Pharmaceutical compositions of the present invention can be administered to a subject by appropriately selecting a suitable administration route depending on the dosage form. The administration route is not particularly limited, but examples include oral administration, intradermal, subcutaneous, intramuscular, intraosseous, peritoneal and intravenous injection, and such. Furthermore, while either systemic administration or local administration near the diseased site is possible, local administration is preferred. More specifically, pharmaceutical compositions of the present invention can be administered by means of injection and such to the cancer tissue or to its vicinity. Alternatively, pharmaceutical compositions of the present invention can be administered surgically into the cancer tissue or to its vicinity. Pharmaceutical compositions of the present invention can also be prepared as a controlled-release preparation by combining them with appropriate carriers.

Dosage interval of pharmaceutical compositions of the present invention may also be appropriately selected according to the age, gender, body weight, and body surface area of subjects for administration, the disease type and such, as well as the dosage form, administration route, and such of the pharmaceutical compositions of the present invention. Examples of the dosage interval include every day, every four days, and every seven days, but are not limited thereto.

Dosage of pharmaceutical compositions of the present invention may also be appropriately selected according to the age, gender, body weight, and body surface area of subjects for administration, the disease type and such, as well as the dosage form, administration route, and such of the pharmaceutical compositions of the present invention.

Examples of the dosage of peptides or salts thereof of the present invention include, for example, 0.001 mg/kg/day to 1000 mg/kg/day, 0.005 mg/kg/day to 500 mg/kg/day, 0.01 mg/kg/day to 250 mg/kg/day, but are not limited thereto.

Pharmaceutical compositions of the present invention may be used in combination with other pharmaceuticals depending on the condition of the administration subjects. The pharmaceuticals used in combination are not particularly limited, but when the pharmaceutical compositions are used for estrogen receptor-positive cancers, examples may include hormone therapy agents such as selective ERα modulators (e.g., tamoxifen and raloxifene), ERα down-regulators (e.g., fulvestrant), aromatase inhibitors, LH-RH agonist formulations, and progesterone formulations. Among these hormone therapy agents, particularly preferred examples include tamoxifen and fulvestrant.

When pharmaceutical compositions of the present invention are used for cancer treatment, one may examine whether the cancer to be treated is accompanied by expression of BIG3 and PHB2 before administering the pharmaceutical compositions. Whether BIG3 and PHB2 are expressed in the cancer to be treated can be confirmed by detecting transcription products or translation products of these genes in the samples collected from the subjects. Known methods can be used for detection methods, and for example, methods of detecting transcription products using probes or PCR methods (for example, cDNA microarray method, Northern blotting, and RT-PCR) and methods of detecting translation products using antibodies and such (for example, Western blotting and immunostaining) may be used.

The present invention also provides articles of manufacture or kits that comprise a pharmaceutical composition of the present invention. The articles of manufacture or kits of the present invention can include a container that houses the pharmaceutical composition of the present invention. An example of an appropriate container includes a bottle, a vial or a test tube, but is not limited thereto. The container may be formed of various materials such as glass or plastic. A label may be attached to the container, and the disease or disease state to which the pharmaceutical composition of the present invention should be used may be described in the label. The label may also indicate directions for administration and such.

The articles of manufacture or kits of the present invention may further comprise a second container that houses pharmaceutically acceptable diluents optionally, in addition to the container that houses the pharmaceutical composition of the present invention. The articles of manufacture or kits of the present invention may further comprise the other materials desirable from a commercial standpoint and the user's perspective, such as the other buffers, diluents, filters, injection needles, syringes, and package inserts with instructions for use.

As needed, the pharmaceutical composition of the present invention can be provided in a pack or dispenser device that can contain one or more units of dosage forms containing active ingredients. The pack can include, for example, a metallic foil or a plastic foil such as a blister pack. Instructions for administration can be attached to the pack or dispenser device.

In another embodiment, the present invention provides the following uses, methods, and such:
(a) uses of the peptide or salt thereof, or polynucleotide encoding the peptide of the present invention in the manufacture of a pharmaceutical composition for either or both of treatment and prophylaxis (prevention) of cancer;
(b) the peptides or salts thereof, or polynucleotides encoding the peptides of the present invention for use in either or both of treatment and prophylaxis (prevention) of cancer;
(c) methods or processes for manufacturing a pharmaceutical composition for either or both of treatment and prophylaxis (prevention) of cancer, the method or process comprising formulating the peptide or salt thereof, or polynucleotide encoding the peptide of the present invention and a pharmaceutically acceptable carrier(s);
(d) methods or processes for manufacturing a pharmaceutical composition for either or both of treatment and prophylaxis (prevention) of cancer, the method or process comprising mixing the peptide or salt thereof, or polynucleotide encoding the peptide of the present invention with a pharmaceutically acceptable carrier(s); and
(e) methods for either or both of treatment and prophylaxis (prevention) of cancer, the method comprising administering the peptide or salt thereof, or polynucleotide encoding the peptide of the present invention to a subject.

In the above uses, methods and such, cancer is preferably BIG3-positive cancer and may be estrogen receptor-positive cancer or estrogen receptor-negative cancer (for example, triple-negative breast cancer). A preferred example of such cancer includes breast cancer.

Hereinbelow, the present invention is described in more detail with reference to the Examples. Nevertheless, while the following materials, method and Examples may serve to assist one of ordinary skill in making and using certain embodiments of the present invention, there are only intended to illustrate aspects of the present invention and thus in no way to limit the scope of the present invention. One of ordinary skill in the art can use methods and materials similar or equivalent to those described herein in the practice or testing of the present invention.

All prior art documents cited herein are incorporated by reference in the present specification.

### EXAMPLE

### [Example 1] Effects on Estrogen-dependent Breast Cancer

### 1. Materials and Methods

Cell lines and Culturing Conditions

Human breast cancer cell line MCF-7 was purchased from JCRB Cell Bank (Osaka, Japan) and maintained in MEM (Thermo Fisher Scientific) supplemented with 10% FBS (Nichirei Biosciences Inc., Tokyo, Japan), 1% Antibiotic/Antimycotic solution (Thermo Fisher Scientific, Waltham, MA, USA), 0.1 mM NEAA (Thermo Fisher Scientific), 1 mM sodium pyruvate (Thermo Fisher Scientific), and 10 μg/mL insulin (Sigma, St. Louis, MO, USA) under 5% CO₂ at 37° C.

Normal mammary gland epithelial cell line MCF-10A was purchased from American Type Culture Collection (ATCC, Manassas, VA, USA), and maintained in MEBM (Lonza) supplemented with a Single Quots kit (BPE, hydrocortisone, hEGF, insulin, gentamycin/amphoterin-B) (Lonza, Walkersville, MD, USA) and 100 ng/mL cholera toxin under 5% CO₂ at 37° C.

### Cell Growth Assay

Growth assay on MCF-7 was carried out by seeding cells into 48-well plates (2×104 cells/200 μL). First, on the next day after seeding, the medium was changed to phenol red-free DMEM/F12 (Thermo Fisher Scientific) supplemented with 10% FBS, 1% Antibiotic/Antimycotic solution, 0.1 mM NEAA, 1 mM sodium pyruvate, and 10 g/mL insulin. 24 hours later, the cells were treated with 10 nM 17β-estradiol (estrogen, Sigma) alone or with 10 nM estrogen and a PHB2 sequence-derived peptide. Growth assay on MCF-10A was carried out by seeding cells into 48-well plates (2×10⁴ cells/200 μL). 24 hours after seeding, PHB2 sequence-derived peptide was added. Growth assays were carried out using the Cell Counting Kit-8 (CCK-8) (Dojindo, Kumamoto, Japan). The data are shown by mean±standard deviation of three independent experiments.

### Antibodies and Immunoblot Analyses

For immunoblot analyses, after performing SDS-PAGE, the membranes blotted with proteins were blocked with 4% BlockAce solution (Dainippon Pharmaceutical, Osaka, Japan) for 3 hours and then incubated to react for 12 hours with antibodies against BIG3 (1:1,000), PHB2 (1:1,000) (Abcam, Cambridge, UK) and phosphorylated PHB2 (Ser39, Scrum, Tokyo, Japan). After allowing interaction with HRP-labeled secondary antibodies (anti-rat IgG-HRP for BIG3, 1:5,000; and anti-rabbit IgG-HRP for PHB2 and phosphorylated PHB2, 1:1,000) (Santa Cruz Biotechnology, Dallas, TX, USA) for 1 hour, the blots were developed with the Enhanced Chemiluminescence (ECL) system (GE Healthcare, Buckinghamshire, UK) and scanned using the Image Reader LAS-3000 mini (Fujifilm, Tokyo, Japan).

### Immunoprecipitation

For immunoprecipitation, MCF-7 was seeded into 10 cm dishes (2×10⁶ cells/10 mL), and like cell growth assay, MCF-7 was treated with 10 nM estrogen alone or with 10 nM estrogen and PHB2 sequence-derived peptide. For immunoprecipitation analysis, cell lysates lysed in a cell lysis buffer (50 mM Tris-HCl; pH 8.0, 150 mM NaCl, 0.1% NP-40, and 0.5% CHAPS; 0.1% protease inhibitor cocktail III) were pre-cleared with a rat IgG antibody and rec-Protein G Sepharose 4B (Thermo Fisher Scientific) at 4° C. for 3 hours. Then, the supernatants were incubated for reaction with 5 μg of an antibody against BIG3 at 4° C. for 12 hours. Next, the antigen-antibody complexes were precipitated using rec-Protein G Sepharose 4B at 4° C. for 1 hour. The immunoprecipitated protein complexes were washed four times with the cell lysis buffer. Then, SDS-PAGE and immunoblot analyses were carried out.

### Peptide Synthesis

All peptides were synthesized by the Fmoc solid-phase synthesis method. NovaSyn TGR resin (0.25 mmol amine/g) or Rink Amide AM resin (0.62 mmol amine/g) was used as resin and a manual Fmoc solid-phase synthesis method was utilized. The Fmoc group was removed by the following procedures: reacting with 20% (v/v) piperidine/DMF solution at room temperature for 10 minutes, the resin was washed five to ten times with DMF, and then three equivalents of an Fmoc amino acid was subjected to coupling in a DMF solvent at room temperature for 90 minutes using N,N-diisopropylcarbodiimide (DIPCDI; 3.0 equivalents) and 1-hydroxy benzotriazole hydrate (HOBt·H2O; 3.3 equivalents) or using N,N-diisopropylethylamine (DIPEA; 3.0 equivalents) and N,N,N,N-tetramethyl-O-(benzotriazole-1-yl)uronium hexafluorophosphate (HBTU; 2.9 equivalents). After washing with DMF, methanol, and ethanol and drying, the protected peptide resin was reacted with a cocktail of TFA: thioanisole: m-cresol: 1,2-ethanedithiol: water (80:5:5:5:5) in the proportion of 100 mg of the protected peptide resin to 5 mL of the cocktail at room temperature for 90 minutes. After TFA was concentrated by nitrogen stream, the residual was precipitated by adding ether, and the precipitate was washed with ether and then dissolved in an appropriate aqueous solvent to perform preparative purification by HPLC.

### 2. Results

### Screening of PHB2 Sequence-Derived Peptides

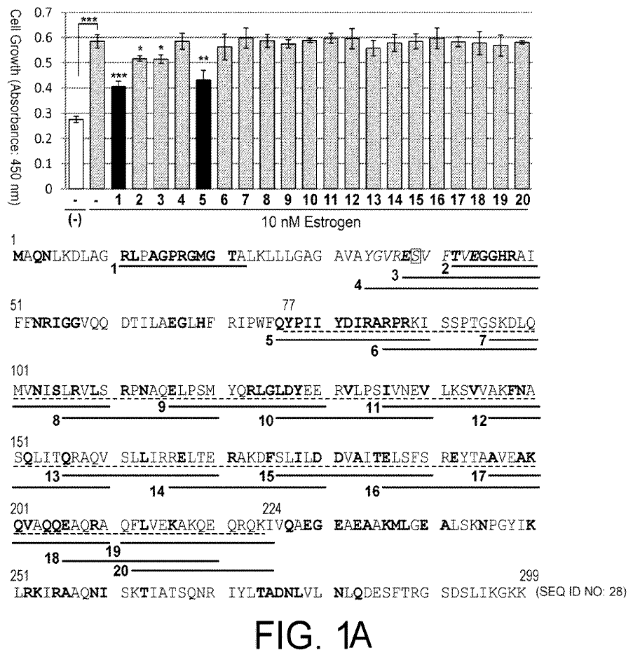

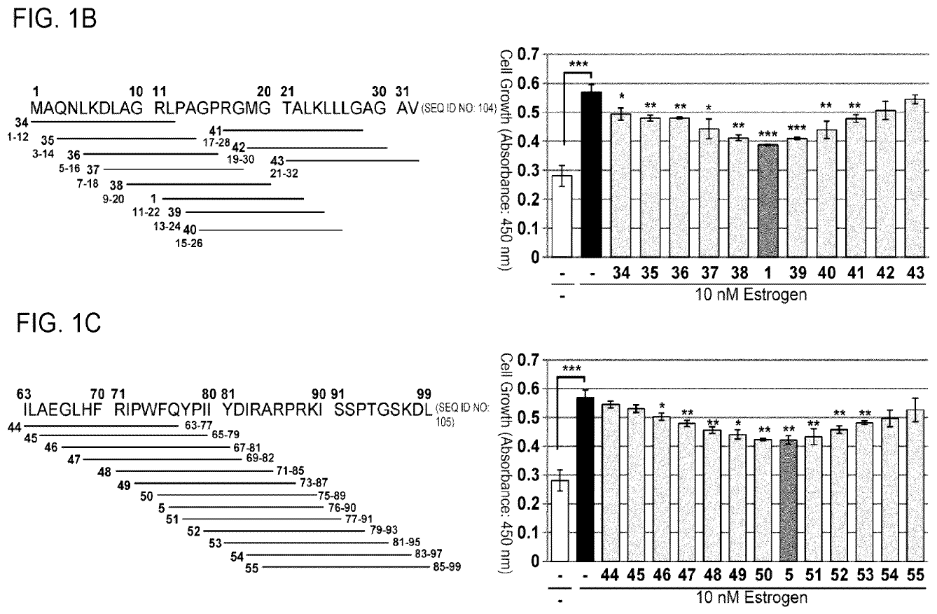

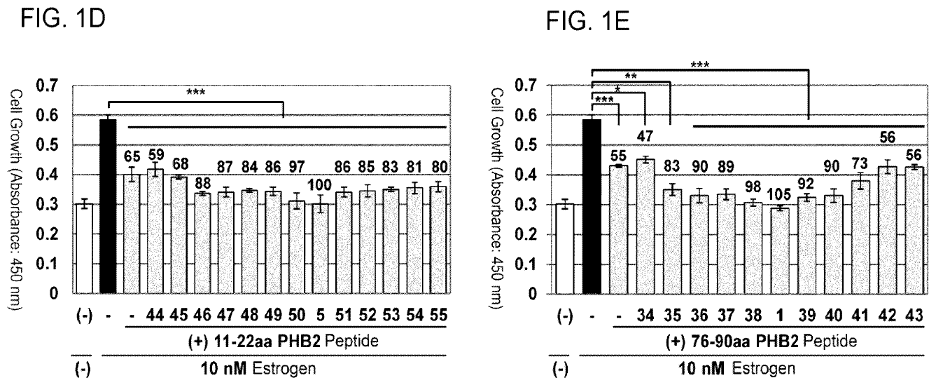

The 20 types of PHB2 protein sequence-derived peptides depicted in FIG. 1A were used to examine the suppressive effects on estrogen (E2)-dependent growth of MCF-7 (treated with 10 μM each peptide for 24 hours). As a result, growth of MCF-7 was significantly promoted by E2 stimulation, while treatments with PHB2-derived peptides No. 1 (11-22aa; SEQ ID NO: 1) and No. 5 (76-90aa; SEQ ID NO: 5) respectively showed the effect of significantly suppressing the E2-dependent growth by about 50% (No: 1: suppression ratio of 58%; No. 5: suppression ratio of 49%). Both peptides almost coincided with the BIG3-binding region predicted by in silico analysis (amino acids in bold letters). Furthermore, peptides No. 2 (42-50aa; SEQ ID NO: 2) and No. 3 (38-50aa; SEQ ID NO: 3) also showed the effect of suppressing the E2-dependent growth by 22% and 23%, respectively. However, each PHB2 sequence-derived peptide had a lower suppressive effect on the E2-dependent growth compared to that of ERAP, suggesting that PHB2 may have multiple BIG3-binding regions.

Next, additional PHB2 sequence-derived peptides surrounding No. 1 (11-22aa) and No. 5 (76-90aa) were synthesized (FIGS. 1B and 1C), and the effect of treatment with 10 μM each peptide for 24 hours on the E2-dependent growth was examined. As a result, among the peptides around No. 1 (11-22aa), the PHB2 (11-22aa) peptide showed the highest suppression ratio (63%), while the suppressive effect attenuated with distance from there (FIG. 1B). On the other hand, among the peptides around No. 5 (76-90aa), the PHB2 (76-90aa) peptide showed the highest suppression ratio (51%), and No. 50 (75-89aa) also had almost the same suppressive effect; however, similarly to the above, the suppression ratio decreased with distance from these peptides (FIG. 1C). What is common to these data is that they comprise each amino acid of 11-21aa and 76-88aa, which showed a high score among the BIG3-binding sites predicted by in silico analysis, and the data suggested that there are two BIG3-binding sites in PHB2.

Thus, the combinations of No. 1 (11-22aa) with peptides surrounding No. 5 (76-90aa) and the combinations of No. 5 (76-90aa) with peptides surrounding No. 1 (11-22aa) were examined for suppressive effect on the E2-depentent growth of MCF-7. As a result, treatment with peptide No. 1 (11-22aa) alone almost reproduced the result with a suppression ratio of 65% for the E2-dependent growth, and when this peptide was used in combination with PHB2 peptides surrounding No. 5 (76-90aa), the combinations with No. 5 (76-90aa) and with No. 50 (75-89aa) showed almost complete suppressive effects of 100% and 97%, respectively (FIG. 1D). Similarly, treatment with No. 5 (76-90aa) alone showed a suppression ratio of 55%, and when this peptide was used in combination with PHB2 peptides surrounding No. 1 (11-22aa), the combination with No. 1 (11-22aa) almost completely suppressed the growth, and the combinations with any of the peptides consisting of a region of 5 to 26 aa (Nos. 36, 37, 38, 39, and 40) showed a suppression ratio of 90% or higher (FIG. 1E). This suggested the importance of the PHB2 regions of No. 1 (11-22aa) and No. 5 (76-90aa) for the binding to BIG3 and the necessity to develop dominant-negative peptides considering these regions.

### Suppressive Effects of PHB2 Sequence-Derived Peptides on E2-Dependent Growth

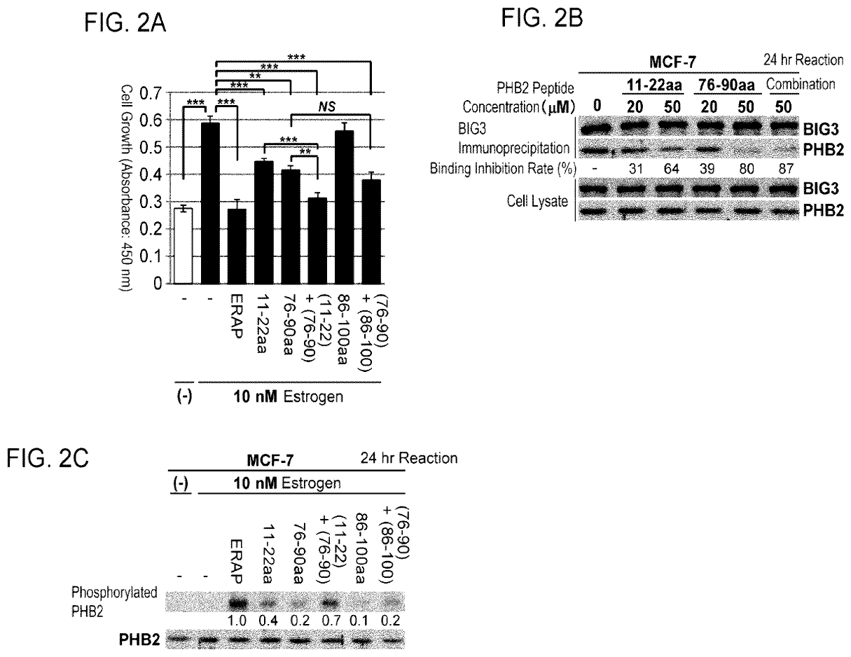

Next, the suppressive effect on E2-depentent growth of MCF-7 was examined when the two types of PHB2 sequence-derived peptides (No. 1 and No. 5), which can suppress E2-depentent growth of MCF-7, were used in combination. As a result, compared to each treatment with No. 1 or No. 5 alone, the use of both peptides in combination enhanced the effect and showed the growth suppressive effect of 88% (FIG. 2A). Moreover, enhanced suppressive effects were not observed when No. 5 was treated in combination with No. 6 (86-100aa), which did not show suppressive effect (suppression ratio of about 10%) in treatment with No. 6 alone. This suggested that there may be a BIG3-binding region in each of 11-22 aa and 76-90 aa of PHB2.

Next, the inhibition of the binding between BIG3 and PHB2 by treatment with 20 μM and 50 μM of No. 1 or No. 5 was examined by immunoprecipitation with a BIG3 antibody. As a result, No. 1 and No. 5 both inhibited the binding between BIG3 and PHB2 in a concentration-dependent manner, and No. 1 and No. 5 showed inhibition ratios of 64% and 80%, respectively, at 50 μM (FIG. 2B). Furthermore, the use of both peptides at 50 μM in combination achieved the inhibition ratio of 87% (FIG. 2B). Next, the effect of each peptide (Nos. 1, 5, and 6) on Ser39 phosphorylation of PHB2 was examined. Compared to Ser39 phosphorylation of PHB2 by treatment with ERAP, a positive control, each treatment with No. 1 or No. 5 alone showed only 40% or 20% band intensity of phosphorylation (FIG. 2C), and even the use of both peptides in combination showed 70% intensity of phosphorylation (FIG. 2C). On the other hand, No. 6 showed 10% band intensity of PHB2 phosphorylation and could hardly induce the phosphorylation (FIG. 2C). Moreover, since even the use of No. 5 and No. 6 in combination showed 20% band intensity, it was suggested that the binding between PHB2 and BIG3 extends over multiple regions of No. 1 and No. 5.

### Suppressive Effect of a Novel Peptide (11-90Aa) Covering the BIG3-Binding Regions of PHB2 Peptides No. 1 and No. 5 on E2-Dependent Growth

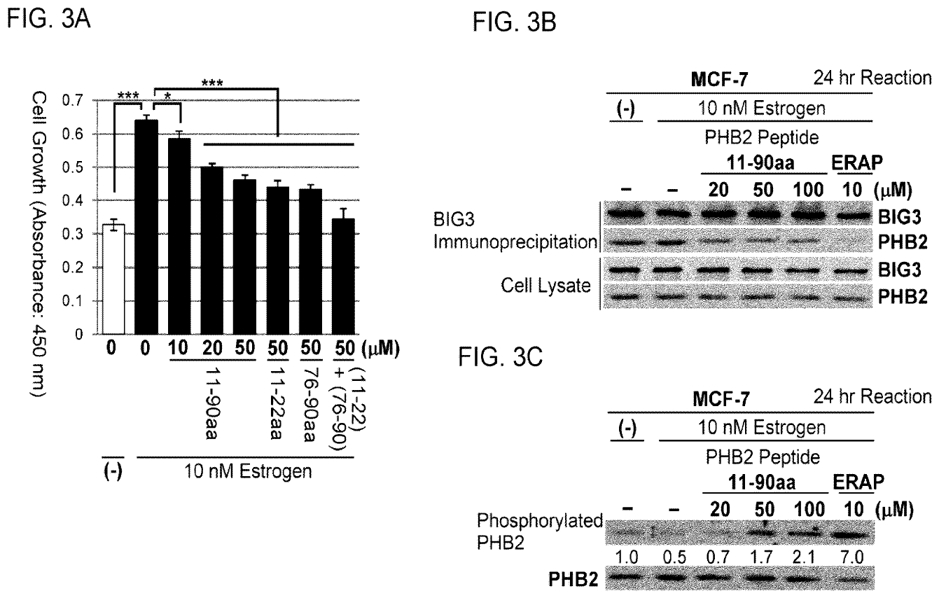

Since the PHB2 sequence-derived peptides (No. 1 and No. 5) can suppress E2-dependent growth and can induce Ser39 phosphorylation of PHB2 by only 50%, a PHB2 peptide of 11-90aa comprising these two regions was newly synthesized and its effect on E2-dependent growth of MCF-7 was examined. As a result, the PHB2 peptide 11-90aa suppressed MCF-7 which proliferated twofold by 24 hour-E2 stimulation in a concentration-dependent manner, but had a suppression ratio of only 57% even at 50 μM (FIG. 3A), which ratio was almost the same as that of No. 1 and No. 5 peptides.

It was then evaluated whether the peptide 11-90aa can inhibit the binding between BIG3 and PHB2 and can induce PHB2 phosphorylation. As a result, the PHB2 peptide 11-90aa inhibited the binding between BIG3 and PHB2 greater than an untreated sample, but did not provide a sufficient effect of inhibiting the binding (FIG. 3B). Furthermore, Ser39 phosphorylation of PHB2 was induced in a manner dependent on the concentration of PHB2 peptide 11-90aa, but it was 30% of the phosphorylation obtained by ERAP treatment, and PHB2 peptide 11-90aa could not induce sufficient phosphorylation (FIG. 3C). This may be because the number of amino acids was large, i.e. 80, and thus the binding to the xx-helical structure of BIG3 was insufficient.

### Suppressive Effects of Linear, Branched, and Cyclic PHB2 on E2-Dependent Growth

Since the combination of PHB2 sequence-derived peptides 11-22aa and 76-90aa enhanced suppression of estrogen-dependent growth to achieve a suppression ratio of 88% (FIG. 2A), a peptide in which both sequences were synthesized linearly (linear bound type; FIG. 4A), a peptide in which both sequences were synthesized with branching (branched bound type; FIG. 4B), and peptides in which each sequence was synthesized in a cyclic form for stabilizing structure and improving membrane permeability (cyclic type; FIG. 4C) were additionally prepared, and the enhancement of the growth suppressive effect of each peptide was examined (treated with 10 μM each peptide for 24 hours). As a result, the linear bound and branched bound PHB2 peptides, in which both sequences were linked via a PEG (polyethylene glycol) sequence, showed suppression ratios of 71% and 57%, respectively. These suppression ratios were enhanced compared to that of administration of a single linear peptide, but were lower than that of the use of the peptides in combination (FIG. 5A). Furthermore, cyclic PHB2 peptides of 11-21aa and 76-88aa showed enhanced suppression ratios compared to non-cyclic peptides (FIG. 5A). Moreover, the use of the cyclic peptides in combination achieved an almost complete suppressive effect (suppression ratio of 96%; FIG. 5A), but an apoptosis-like phenomenon in which cells floated could not be confirmed.

Next, the effects of the cyclic peptides on growth of normal mammary gland epithelial cells, MCF-10A, which do not express ERα and BIG3, were examined (treated with 10 μM each peptide for 24 hours). As a result, although little suppressive effects were observed in the linear bound and cyclic bound PHB2 peptides (FIG. 5B; linear bound type: inhibition ratio of 10%, cyclic 11-21aa: inhibition ratio of 14%, cyclic 76-88aa: inhibition ratio of 15%), it was suggested that most PHB2 peptides specifically suppress E2-dependent growth with little effect on MCF-10A growth.

Subsequently, it was examined whether these PHB2 peptides can inhibit the interaction between BIG3 and PHB2. As a result, BIG3 strongly bound to PHB2 when the cells were untreated or stimulated with E2 (FIG. 5C) and treatment with each PHB2 peptide alone could hardly inhibit the interaction between BIG3 and PHB2 (FIG. 5C); however, the respective combination of the linear PHB2 peptides and the cyclic PHB2 peptides could markedly inhibit the interaction (FIG. 5C; combination of linear type: inhibition ratio of 67%, combination of cyclic type: inhibition ratio of 81%), and these results suggested that there are two BIG3-binding regions on PHB2. On the other hand, it was judged that the linear bound and branched bound PHB2 peptides cannot cover two BIG3-binding regions on PHB2.

### Long-Term Stability of the Suppressive Effects of Cyclic PHB2 Peptides on E2-Dependent Growth

Since it was suggested that cyclic PHB2 peptides may acquire low concentration and long-term stability due to enhanced membrane permeability and fixation of structure, the long-term stability for up to 96 hours was examined when cells were treated with 10 μM each cyclic PHB2 peptide alone. As a result, the linear PHB2 peptides 11-22aa and 76-90aa showed suppression ratios of 40% and 61%, respectively, in a 24-hour treatment, but exhibited significantly decreased suppression ratios of 31% and 24%, respectively, after 96 hours (FIG. 6A). Meanwhile, the cyclic peptide 11-21aa and cyclic peptide 76-88aa showed suppression ratios of 67% (53% in a 24-hour treatment) and 72% (58% in a 24-hour treatment), respectively, even after 96 hours (FIG. 6A), and their suppressive effects stably lasted for up to 96 hours. It was considered from these data that the suppressive effects of the cyclic peptides could last for a long time due to stable fixation of their tertiary structure similar to the cross-linked peptides.

Next, since the cyclic PHB2 peptides could sustain their suppressive effects for up to 96 hours, the effect on growth of MCF-10A, which does not express ERα and BIG3, was examined by treating with 1 μM or 10 μM each cyclic PHB2 peptide for up to 96 hours. As a result, the cyclic peptides of PHB2 sequences 11-21 aa and 76-88 aa each had little effect at 1 μM (both of them had suppression ratios of 5% to 7%), but showed suppression ratios of 10% to 15% at 10 μM (FIG. 6B), suggesting that they have a small non-specific suppressive effect. However, it was considered that the suppression of E2-dependent growth of MCF-7 by the cyclic PHB2 peptides was mainly due to the inhibition of the binding between BIG3 and PHB2.

### Concentration-Dependent Suppressive Effect of Cyclic PHB2 Peptides on E2-Dependent Growth

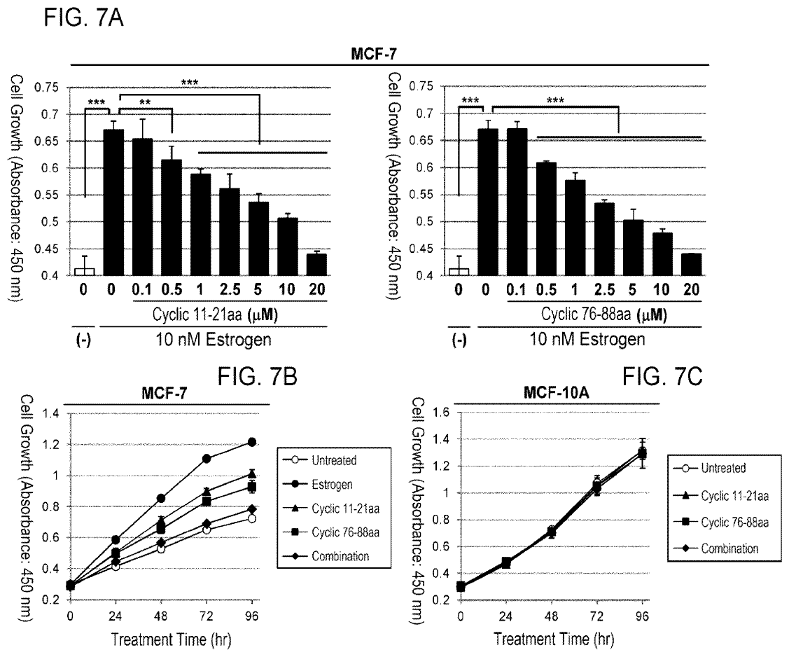

The 50% inhibition concentrations (IC50) of the cyclic PHB2 peptides for E2-dependent growth of MCF-7 were calculated and a synergistic suppressive effect of the peptides at the IC50 was examined. As a result, each cyclic PHB2 peptide suppressed E2-dependent growth in a concentration-dependent manner, and the cyclic 11-21aa and cyclic 76-88aa showed IC50 values of 4.06 μM and 2.11 μM, respectively (FIG. 7A). Then, 4 μM cyclic 11-21aa and 2 μM cyclic 76-88aa were used to examine the effect of long-term combination, and the use of peptides in combination for 24 hours showed a synergistic suppressive effect of 82%, which effect lasted for up to 96 hours (FIG. 7B; combination: suppression ratio of 88%, cyclic 11-21aa: suppression ratio of 41%, cyclic 76-88aa: suppression ratio of 59%). Furthermore, the peptides at these concentrations had little effect on growth of MCF-10A (FIG. 7C).

Identification of Amino Acids in PHB2 Peptide Sequences that are Important for the Binding to BIG3

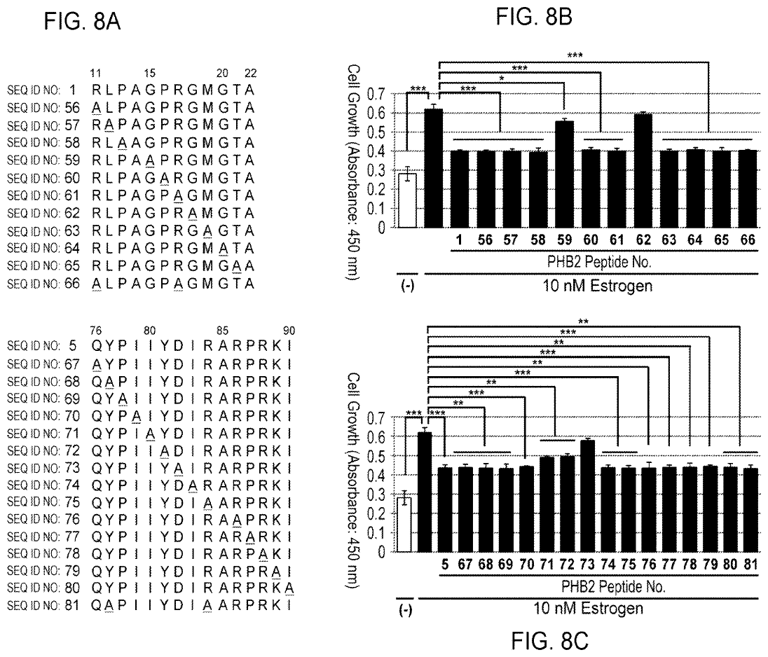

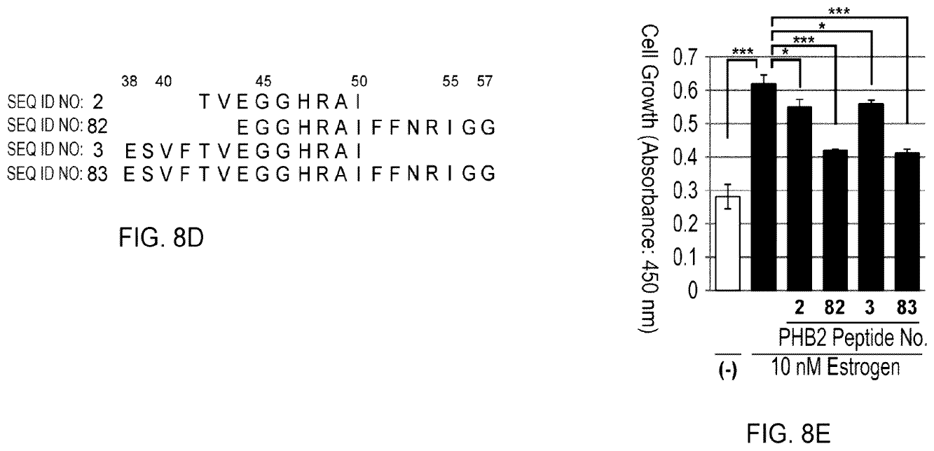

Since PHB2-derived peptides No. 1 (11-22aa: SEQ ID NO: 1) and No. 5 (76-90aa: SEQ ID NO: 5) had a suppressive effect by about 50% on E2-dependent growth, peptides were made in which each amino acid in the peptide sequences of No. 1 and No. 5 was mutated to alanine (FIG. 8A) to identify amino acids important for growth suppression. In the experiment, MCF-7 was seeded, 10 μM each PHB2 peptide and 10 nM estrogen were added 48 hours after seeding, and the cell number was monitored after another 24 hours. First, the amino acids of PHB2 sequence 11-22 aa were evaluated and No. 1 (11-22aa) suppressed estrogen-dependent cell growth by up to 65%, while only alanine-mutated peptides No. 59 and No. 62 (SEQ ID NOs: 59 and 62) attenuated suppression ratios of 19% and 8%, respectively (FIG. 8B). On the other hand, since other alanine-mutated peptides showed almost the same suppression ratio as No. 1 (FIG. 8B), glycines at positions 15 and 18 were considered to be important for the binding to BIG3, suggesting that the suppressive activity may be improved by converting these positions into an isomeric form, a D-amino acid.

Next, the amino acids of PHB2 sequence 76-90 aa were evaluated. Although the suppression ratio of No. 5 (76-90aa) on estrogen-dependent growth was 54% and nearly reproduced (FIG. 8C), the suppression ratios of alanine-mutated peptides Nos. 71 to 73 (SEQ ID NOs: 71 to 73) attenuated below No. 5 (76-90aa) and were 38%, 37%, and 13%, respectively (FIG. 8C), and particularly aspartic acid at position 82 was considered to be necessary for the binding between BIG3 and PHB2.

Furthermore, peptides (FIG. 8D; Nos. 82 and 83 (SEQ ID NOs: 82 and 83)) were prepared by adding 51-57aa comprising 53-57 aa, which was predicted to be involved in the interaction between BIG3 and PHB2 by in silico analysis (FIG. 1A), to No. 2 (42-50aa: SEQ ID NO: 2) and to No. 3 (38-50aa: SEQ ID NO: 3), which showed a suppressive effect other than peptides No. 1 (11-22aa) and No. 5 (76-90aa), and these peptides were used to examine the effects on estrogen-dependent cell growth. As a result, while the suppression ratios of No. 2 and No. 3 were 20% and 17%, respectively, peptides No. 82 and No. 83, to which the amino acids at positions up to 57 were added, showed improved suppression ratios of 59% and 61%, respectively (FIG. 8E). Thus, it was suggested that by having the amino acids from glutamic acid at position 44 to glycine at position 57, the PHB2 peptides can be comparable in suppression ratio to No. 1 and No. 5.

### [Example 2] Effects on Triple-Negative Breast Cancer

### 1. Materials and Methods

Cell lines and Culture Conditions

Human breast cancer cell line MDA-MB-231 was purchased from American Type Culture Collection (ATCC, Manassas, VA, USA), and cultured using Leibovitz's L-15 medium (Thermo Fisher Scientific) supplemented with 10% FBS (Thermo Fisher Scientific, Waltham, MA, USA) and a 1% antibiotic/antimycotic solution (Wako Pure Chemical, Osaka, Japan) at 37° C. without CO₂ concentration control.

### Cell Growth Assay

MDA-MB-231 cells were seeded into 48-well plates at a cell density of 1×10⁴ cells/200 μL in well. 48 hours later, the medium in each well was exchanged to a medium supplemented with PHB2 peptides 11-22aa or 76-90aa (three-fold serial dilution from 20 μM), and after culturing for another 96 hours, the level of cell growth was measured using the Cell Counting Kit-8 (Dojindo, Kumamoto, Japan). Data were obtained from three independent experiments, a graph (mean±standard deviation) was produced using a graphing and data analyzing software SigmaPlot (Systat Software, San Jose, CA, USA), and the 50% inhibition concentration (IC50) of the peptide against cell growth was calculated.

### Combination Assay

MDA-MB-231 cells were seeded into 48-well plates at a cell density of 1×10⁴ cells/200 μL in well. 48 hours later, the medium in each well was exchanged to media supplemented with PHB2 peptide 11-22aa (added concentration: IC50 value), PHB2 peptide 76-90aa (added concentration: IC50 value) and a mixed solution of both peptides (added concentration: respective IC₅₀ value), respectively, or to a medium supplemented with phosphate buffered saline (PBS) as a negative control. After culture for another 96 hours, the cell growth level was measured using the Cell Counting Kit-8 (Dojindo, Kumamoto, Japan). The data obtained was used to calculate relative values based on the growth level when PBS was added, and the graph was produced. 2. Results

### Growth Suppressive Effects of PHB2 Peptides on a Breast Cancer Cell Line

To examine the cell growth suppressive effects of PHB2 peptides 11-22aa and 76-90aa on the breast cancer cell line MDA-MB-231, serial dilution series of the peptides were prepared and the growth level was measured 96 hours after addition to the cells. As a result, as shown in FIGS. 9A and 9B, concentration-dependent suppressive effects on cell growth were observed in both peptides. The 50% inhibition concentrations (IC50) were 0.462 μM in the peptide 11-22aa and 0.273 μM in the peptide 76-90aa, and the peptide 76-90aa showed a more potent growth suppressive effect.

### Effect of Use of PHB2 Peptides 11-22Aa and 76-90Aa in Combination

To examine the effect of use of PHB2 peptides 11-22aa and 76-90aa in combination on suppression of cell growth, the breast cancer cell line MDA-MB-231 was used to compare the cell growth levels when both peptides were mixed and added at respective IC50 and when each peptide was added alone at respective IC50. As a result, as shown in FIG. 9C, the addition of each peptide alone showed the suppression of growth by about 50% compared to when phosphate buffered saline (PBS), a negative control, was added; however, the combination enhanced the suppressive effect by about 62%.

### [Example 3] Effects of Cross-Linking PHB2 Peptide on Estrogen-Dependent Breast Cancer

### 1. Materials and METHODS

### Cell Lines and Culturing Conditions

Human breast cancer cell line MCF-7 was purchased from JCRB Cell Bank (Osaka, Japan) and maintained in MEM (Thermo Fisher Scientific) supplemented with 10% FBS (Nichirei Biosciences Inc., Tokyo, Japan), a 1% Antibiotic/Antimycotic solution (Thermo Fisher Scientific, Waltham, MA, USA), 0.1 mM NEAA (Thermo Fisher Scientific), 1 mM sodium pyruvate (Thermo Fisher Scientific), and 10 μg/mL insulin (Sigma, St. Louis, MO, USA) under 5% CO₂ at 37° C.

Normal mammary gland epithelial cell line MCF-10A was purchased from American Type Culture Collection (ATCC, Manassas, V A, USA), and maintained in MEBM (Lonza) supplemented with a Single Quots kit (BPE, hydrocortisone, hEGF, insulin, gentamycin/amphoterin-B) (Lonza, Walkersville, M D, USA) and 100 ng/mL cholera toxin under 5% CO₂ at 37° C.

### Cell Growth Assay

Growth assay on MCF-7 was carried out by seeding cells into 48-well plates (2×104 cells/200 μL). First, on the next day after seeding, the medium was changed to phenol red-free DMEM/F12 (Thermo Fisher Scientific) supplemented with 10% FBS, 1% Antibiotic/Antimycotic solution, 0.1 mM NEAA, 1 mM sodium pyruvate, and 10 μg/mL insulin. 24 hours later, the cells were treated with 10 nM 17β-estradiol (estrogen, Sigma) alone or with 10 nM estrogen and a PHB2 sequence-derived peptide. Growth assay on MCF-10A was carried out by seeding cells into 48-well plates (2×10⁴ cells/200 μL). 24 hours after seeding, PHB2 sequence-derived peptide was added. Growth assays were carried out using the Cell Counting Kit-8 (CCK-8) (Dojindo, Kumamoto, Japan). The data are shown by mean±standard deviation of three independent experiments.

### 2. Results

### Suppressive Effects of Cross-Linked PHB2 Peptides on Estrogen-Dependent Growth

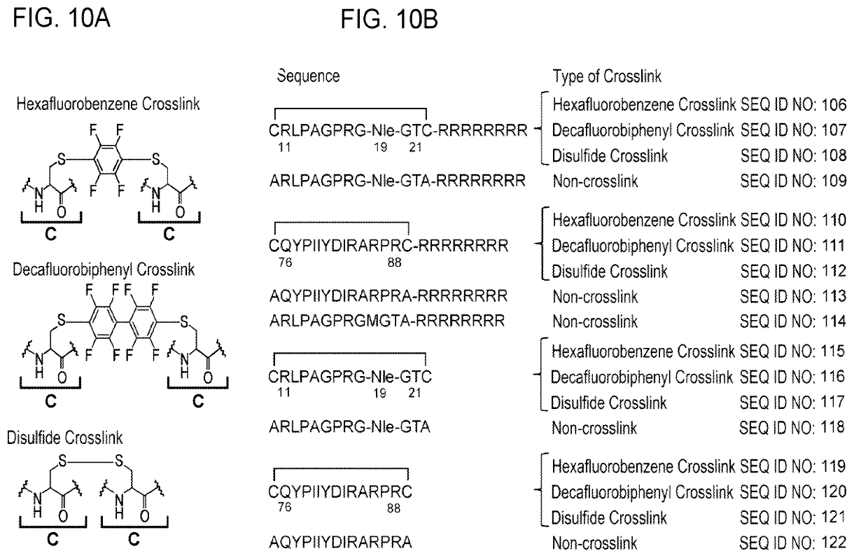

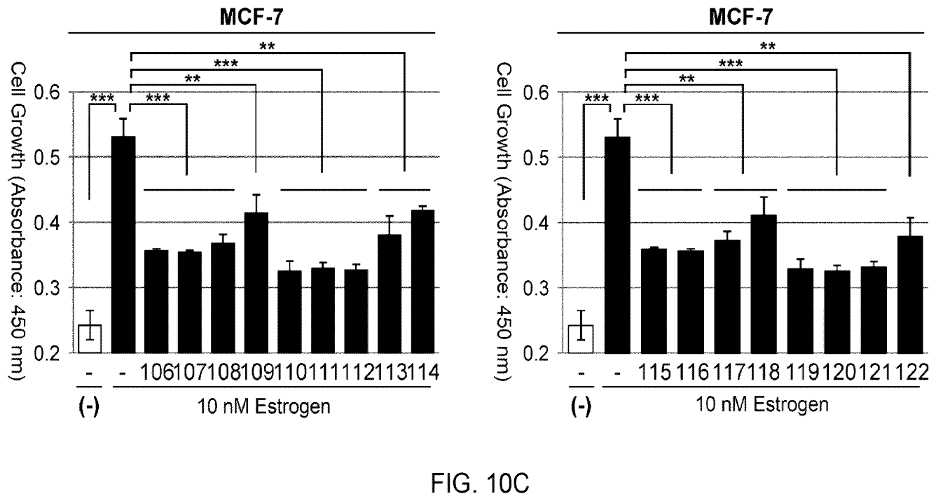

PHB2 peptides 11-21aa and 76-88aa were cross-linked by three types of cross-linking methods (FIG. 10A: hexafluorobenzene cross-linking, decafluorobiphenyl cross-linking, and disulfide cross-linking) to prepare cross-linked PHB2 peptides (stapled PHB2 peptides) (FIG. 10B), and the suppressive effects of these peptides on estrogen-dependent growth were examined. In the experiment, breast cancer cells MCF-7 were seeded, 10 μM each PHB2 peptide and 10 nM estrogen were added after 48 hours, and the cell number was evaluated after another 24 hours by MTT assay.

Each cross-linked PHB2 peptide of 11-21aa and 76-88aa improved the suppressive effect on estrogen-dependent growth about 1.5-fold compared to non-cross-linked PHB2 peptides (SEQ ID NOs: 109, 113, 114, 118, and 122) (polyarginine addition: FIG. 10C (left); without polyarginine: FIG. 10C (right)). The improved effects did not vary depending on the cross-linking method (stapling method). Moreover, cross-linked PHB2 peptides which do not have polyarginine at the C terminus (SEQ ID NOs: 115, 116, 117, 119, 120, and 121) showed a slightly higher suppression ratios than peptides to which polyarginine was added (with polyarginine: suppression ratio of about 60%; without polyarginine: suppression ratio of about 70%), suggesting that polyarginine may block the function of cross-linked structure.

### Suppressive Effects of Cyclic PHB2 Peptides on Estrogen-Dependent Growth

For improving the cell membrane permeability and the structural stability of cyclic PHB2 peptides (FIG. 4C, SEQ ID NOs: 25 and 26), their cross-linking forms were changed and the suppressive effects on estrogen-dependent growth were examined. In addition to disulfide cross-linking (SEQ ID NOs: 25 and 26) so far examined, hexafluorobenzene cross-linking (FIG. 11A; SEQ ID NOs: 123 and 126) and decafluorobiphenyl cross-linking (FIG. 11A; SEQ ID NOs: 124 and 127) were evaluated. As a result, cyclic PHB2 peptides cross-linked using fluorobenzene (FIG. 11A; SEQ ID NOs: 123, 124, 126, and 127) slightly improved the growth suppressive effect compared to disulfide cross-linking (FIG. 11B), and cyclic PHB2 peptide 11-21aa and cyclic PHB2 peptide 76-88aa enhanced the suppression ratios to about 70% and about 80%. Furthermore, no difference has been observed for fluorobenzene cross-linking between a single cross-linking (hexafluorobenzene cross-linking) and a double cross-linking (decafluorobiphenyl cross-linking).

### Effects of Modifications of PHB2 Peptide 11-22Aa on Estrogen-Dependent Growth

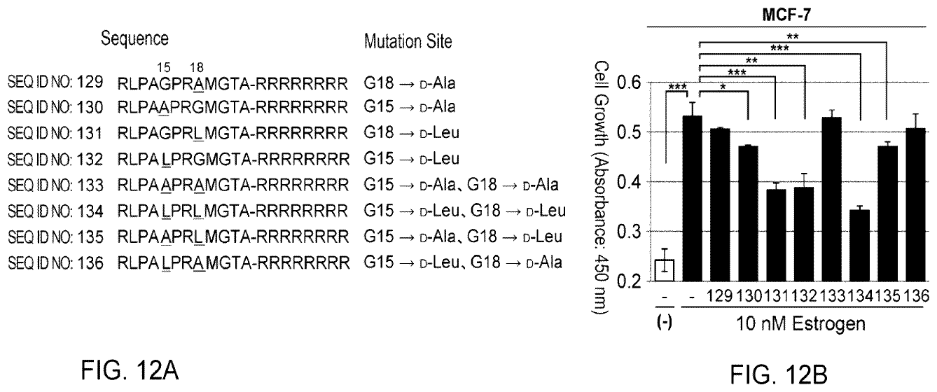

In the PHB2 peptide 11-22aa, glycines at positions 15 and 18 in the amino acid sequence of SEQ ID NO: 28 (full-length PHB2 polypeptide) were considered to be important for the binding to BIG3 (FIG. 8). Then, it was examined whether substitutions of these positions with D-alanine and D-leucine (FIG. 12A) enhance the suppressive activity or not. As a result, while the PHB2 peptide in which glycines at positions 15 and 18 were substituted with D-leucines (SEQ ID NO: 134) showed a suppression ratio of about 65% (FIG. 12B), the substitutions with D-alanines (SEQ ID NO: 133) had no suppressive effect (FIG. 12B). However, since the suppression ratio of PHB2 peptide 11-22aa (SEQ ID NO: 1; FIG. 8B) was about 65%, the substitutions with D-leucine showed little improvement in suppressive effect.

### Effects of Cross-Linked PHB2 Peptides on Growth of Mammary Gland Epithelial Cells

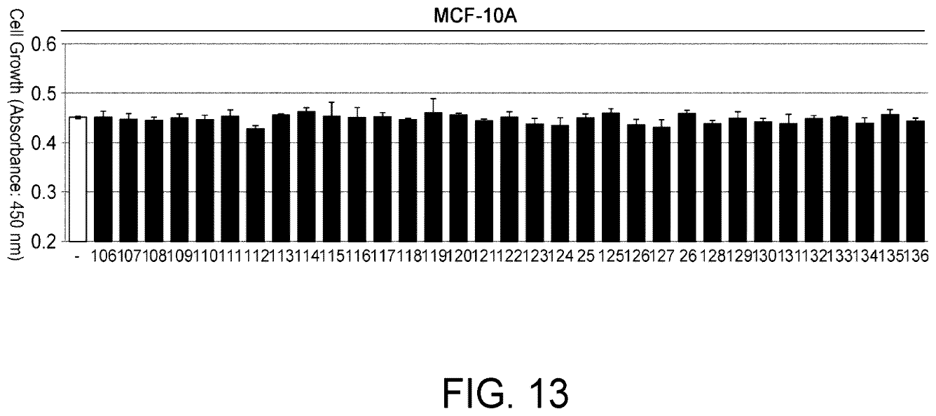

The effects of cross-linked and cyclic PHB2 peptides on growth of normal mammary gland epithelial cells MCF-10A, which do not express ERα and BIG3, were examined (treated with 10 μM each PHB2 peptide for 24 hours). As a result, all the PHB2 peptides evaluated had no effect on growth of MCF-10A (FIG. 13).

### INDUSTRIAL APPLICABILITY

The present invention provides PHB2 amino acid sequence-derived peptides which exert an inhibitory effect on the BIG3-PHB2 interaction and are useful as therapeutic agents for breast cancer. The peptides provided by the present invention are useful in treating cancer such as breast cancer. More specifically, the peptides of the present invention are useful in treating BIG3-positive and/or estrogen receptor-positive cancer. The peptides of the present invention target not PHB2, whose expression is observed in organs throughout the human body, but BIG3, which is a protein highly expressed specifically in particularly estrogen receptor-positive cancer, and thus these peptides can be expected to have high selectivity for estrogen receptor-positive cancer. Moreover, the peptides of the present invention also exert an antitumor effect against triple-negative breast cancer.

1. A peptide, comprising a site binding to a BIG3 polypeptide in a PHB2 polypeptide, wherein the peptide inhibits the binding between the PHB2 polypeptide and the BIG3 polypeptide, and wherein the peptide comprises all or part of the amino acid sequence consisting of the amino acids at positions 76 to 88 in the amino acid sequence of SEQ ID NO: 28.

2. The peptide of claim 1, comprising an amino acid sequence selected from the group consisting of (a) to (b) below:
(a) an amino acid sequence selected from the group consisting of SEQ ID NOs: 26, 5, 47 to 53, 110 to 113, 119 to 122, and 126 to 128; and
(b) an amino acid sequence in which one, two, or several amino acids are substituted, deleted, inserted and/or added in the amino acid sequence selected from the group consisting of SEQ ID NOs: 26, 5, 47 to 53, 110 to 113, 119 to 122, and 126 to 128.

3. The peptide of claim 2, wherein the peptide comprises an amino acid sequence selected from the group consisting of (a′) below:
(a′) an amino acid sequence in which one, two, or several amino acid residues located at positions other than that corresponding to aspartic acid at position 82 in the amino acid sequence of SEQ ID NO: 28 are substituted with other amino acid residues in an amino acid sequence selected from the group consisting of SEQ ID NOs: 26, 5, 47 to 53, 110 to 113, 119 to 122, and 126 to 128.

4. The peptide of claim 1, wherein the peptide consists of 80 amino acid residues or less.

5. The peptide of claim 1, wherein the peptide consists of 25 amino acid residues or less.

6. The peptide of claim 1, wherein the peptide consists of an amino acid sequence selected from the group consisting of SEQ ID NOs: 26, 5, 47 to 53, 110 to 113, 119 to 122, and 126 to 128.

7. The peptide of claim 1, wherein the peptide has been modified with a cell membrane-permeable substance.

8. The peptide of claim 1, wherein the peptide is cyclic.

9. The peptide of claim 1, wherein the peptide is cross-linked.

10. The peptide of claim 1, wherein the peptide has either or both of the following properties (i) and (ii):
(i) suppressing growth of BIG3-positive cells; and
(ii) promoting phosphorylation of a serine residue in the PHB2 polypeptide in BIG3-positive cells.

11. A pharmaceutical composition comprising: at least one ingredient selected from the group consisting of one or more of the peptides of claim 1 and a pharmaceutically acceptable salt(s) of the peptide(s); and a pharmaceutically acceptable carrier.

12. The pharmaceutical composition of claim 11, wherein the composition comprises a peptide comprising all or part of the amino acid sequence consisting of the amino acids at positions 76 to 88 in the amino acid sequence of SEQ ID NO: 28.

13. The pharmaceutical composition of claim 11, which is for suppressing growth of cancer cells or for treatment and/or prophylaxis (prevention) of cancer.

14. The pharmaceutical composition of claim 13, wherein the cancer is BIG3-positive cancer.

15. The pharmaceutical composition of claim 13, wherein the cancer is breast cancer.

16. The pharmaceutical composition of claim 13, wherein the cancer is estrogen receptor-positive cancer.
